SEQUENCE LISTING (18 sequences)

SEQ ID NO: 1 - DNA, 20 bp, Artificial Sequence
  synthetic construct
agaactgtgctgggaagacg

SEQ ID NO: 2 - DNA, 20 bp, Artificial Sequence
  synthetic construct
gccatgccttcaaaatgcca

SEQ ID NO: 3 - DNA, 19 bp, Artificial Sequence
  synthetic construct
aggagtgggtgtgtgacga

SEQ ID NO: 4 - DNA, 20 bp, Artificial Sequence
  synthetic construct
ccaggcagttggctctaatc

SEQ ID NO: 5 - DNA, 20 bp, Artificial Sequence
  synthetic construct
ctcctgcaccaccaactgct

SEQ ID NO: 6 - DNA, 20 bp, Artificial Sequence
  synthetic construct
gggccatccacagtcttctg

SEQ ID NO: 7 - DNA, 20 bp, Artificial Sequence
  synthetic construct
gttgcctgtctcgggaaact

SEQ ID NO: 8 - DNA, 20 bp, Artificial Sequence
  synthetic construct
tacacgctgtgagctccctt

SEQ ID NO: 9 - DNA, 21 bp, Artificial Sequence
  synthetic construct
gggagtcttactgctcatcgg

SEQ ID NO: 10 - DNA, 20 bp, Artificial Sequence
  synthetic construct
tcaaacccttcttgtgggca

SEQ ID NO: 11 - DNA, 20 bp, Artificial Sequence
  synthetic construct
ggaggctgctgaacgaaagt

SEQ ID NO: 12 - DNA, 18 bp, Artificial Sequence
  synthetic construct
gggcgctcttgtagtggg

SEQ ID NO: 13 - DNA, 21 bp, Artificial Sequence
  synthetic construct
ggaccaagtacatgaaccacc

SEQ ID NO: 14 - DNA, 20 bp, Artificial Sequence
  synthetic construct
tgcaggactggtgattggac

SEQ ID NO: 15 - DNA, 20 bp, Artificial Sequence
  synthetic construct
cgagtcggatctggtcatgg

SEQ ID NO: 16 - DNA, 20 bp, Artificial Sequence
  synthetic construct
gacacagcaagctcacaagc

SEQ ID NO: 17 - DNA, 20 bp, Artificial Sequence
  synthetic construct
ccctcgttttgccatgaacc

SEQ ID NO: 18 - DNA, 20 bp, Artificial Sequence
  synthetic construct
gttgctgctggttggagttg

(19) United States
(12) Patent Application Publication
(10) Pub. No.: US 2022/0236255 A1
(43) Pub. Date: Jul. 28, 2022

(54) Self-Contained Responsive Biological Systems and Methods

(71) Applicant: Arizona Board of Regents on Behalf of the University of Arizona, Tucson, AZ (US)

(72) Inventors: Jerome LACOMBE, Phoenix, AZ (US); Ashlee HARRIS, Phoenix, AZ (US); Ryan ZENHAUSERN, Phoenix, AZ (US); Frederic ZENHAUSERN, Phoenix, AZ (US)

(21) Appl. No.: 17/622,020

(22) PCT Filed: Jun. 26, 2020
(86) PCT No.: PCT/US2020/039941

§ 371 Date: Dec. 22, 2021

Related U.S. Application Data
(60) Provisional application No. 62/868,554, filed on Jun. 28, 2019.
(60) Provisional application No. 62/962,323, filed on Jan. 17, 2020.

(51) Int. Cl. G01N 33/50; C12Q 1/6872; C12N 5/09; C12N 5/071

(52) U.S. Cl. CPC G01N 33/5082; C12Q 1/6872; C12N 5/0693; C12N 5/0698; C12N 2501/165; C12N 2533/78; C12N 2513/00; C12N 2533/54; C12N 2533/52; C12N 2533/90

(57) ABSTRACT

A method of simulating a biological response of a cellular system may include removing at least some DNA-containing material from a vascular plant tissue to produce a vascularized cellulose scaffold, seeding the vascularized cellulose scaffold with cultured biological cells, growing cultured biological cells on the vascularized cellulose scaffold to produce the vascularized biological system, subjecting the vascularized biological system to an external stimulus, and measuring a response of the vascularized biological system. In some embodiments, the removing step comprises submerging the plant tissue in a fluid comprising supercritical CO2, peracetic acid and ethanol.

### CROSS-REFERENCE TO RELATED APPLICATIONS

This application claims the benefit of priority to U.S. Provisional Patent Application No. 62/868,554 filed Jun. 28, 2019 and U.S. Provisional Patent Application No. 62/962,323 filed Jan. 17, 2020, each of which is hereby incorporated by reference in its entirety.

### REFERENCE TO A SEQUENCE LISTING

A sequence listing containing SEQ ID NOs: 1-18 is provided herewith in a computer-readable nucleotide/amino acid .txt file, created on Jun. 26, 2020, size of 4 kilobytes, and is specifically incorporated by reference.

### BACKGROUND OF INVENTION

Many in vitro cellular studies are based on cell monolayer cultures and cannot satisfactorily mimic the complex three-dimensional environments and multi-component/constituent structure of biological tissue, including organs, tumors, and soft tissue. The three-dimensional (3D) tumor microenvironment (TME) is heterogeneous in both cellularity (e.g. epithelial, vascular, immune cells, and fibroblasts) and extracellular matrix (ECM) composition.¹ Although many three-dimensional multi-culture systems have been developed, they still suffer from the fundamental disadvantage of not reproducing the complete architecture of the modeled biological system. In vivo animal systems are usually the method of choice, but the important physiological differences between animals and humans make these approaches often inappropriate or inaccurate to mimic the human TME. Additionally, the tumor microenvironment (TME) has a high impact on cancer cell radiation response and, ultimately, patient survival after radiation treatment. Accordingly, there is a need in the art for cell culture methods and systems that better reflect the actual 3D properties, both physical and biological, of a biological tissue. In the context of cancer cell treatment, improved methods of simulating a biological response of a cellular system that address these issues would be beneficial for a range of applications.

### SUMMARY OF THE INVENTION

Provided herein are methods and systems that address the above problems by utilizing a combination of biological tissues, with a first tissue corresponding to a vascular scaffold formed from a substantially decellularized vascular plant tissue (e.g., a leaf) and a second tissue corresponding to a mammalian cell that is adhered to the vascular scaffold. The mammalian cell may correspond to a plurality of cell types. For example, an endothelial cell that typically lines a blood vessel wall and surrounding cell tissue, including cancer cells. The methods and systems provided herein are compatible with a range of tissue cells, depending on the application of interest, such as cellular components of an organ, cancer cells of a tumor, cells of the vasculature, such as endothelial cells and smooth muscle cells, and combinations thereof. The cells may comprise a eukaryotic cell, such as HUVEC cells. Any of the methods and systems provided herein may further comprise extracellular matrix, such as collagen, enzymes, and glycoproteins, that provide structural and biochemical support of surrounding cells, including as a scaffold or matrix.

Any of the methods and systems provided herein are particularly useful and compatible with dynamic control of the cell culture. For example, the vasculature of the plant tissue readily accommodates changes in perfusion rate, localized changes (e.g., spatially varying) in perfusion, and time-varying changes, such as by changing the flow-rate through the plant conduits or periodic changes in the perfusate introduced to the plant conduits. Thus, simulating, in vitro, a biological response of a cellular system.

In one embodiment, a method of producing a vascularized biological system includes removing at least some DNA-containing material from a vascular plant tissue to produce a vascularized cellulose scaffold, preparing the vascularized cellulose scaffold for biological cell attachment, seeding the vascularized cellulose scaffold cultured biological cells, and growing the cultured biological cells on the vascularized cellulose scaffold to produce the vascularized biological system. In this manner, cultured, three-dimensional composite biological systems are formed that more reliably and accurately recapitulate tissue. In some embodiments, the

In one embodiment, the growing comprises three dimensional proliferation of the cultured biological cells. In one embodiment, the growing comprises supplying the cultured biological cells with a growth medium via vessels of the vascularized cellulose scaffold.

In one embodiment, the cultured biological cells comprise cancer cells, such as prostate cancer cells. In one embodiment, the removing step comprises cannulating a vascular structure of the plant tissue, and injecting a liquid comprising a nonionic surfactant through the vascular structure.

In some embodiments, the cultured biological cells comprise 3D

A method of simulating a biological response of a cellular system comprises removing at least some DNA-containing material from a vascular plant tissue to produce a vascularized cellulose scaffold; seeding the vascularized cellulose scaffold with cultured biological cells; growing the cultured biological cells on the vascularized cellulose scaffold to produce a vascularized biological system; subjecting the vascularized biological system to an external stimulus; and measuring a response of the vascularized biological system.

In some embodiments, the removing step comprises treating the plant tissue with a fluid comprising a supercritical fluid. In some embodiments, the removing step comprises submerging the plant tissue in a fluid comprising supercritical CO₂. In some embodiments, the fluid comprises supercritical CO₂, peracetic acid and ethanol.

In one embodiment, the fluid comprises at least 80 wt % supercritical CO₂. In one embodiment, the fluid comprises at least 85 wt % supercritical CO₂. In one embodiment, the fluid comprises at least 90 wt % supercritical CO₂. In one embodiment, the fluid comprises at least 95 wt % supercritical CO₂. In one embodiment, the fluid comprises at least 98 wt % supercritical CO₂. In one embodiment, the fluid comprises at least 99 wt % supercritical CO₂. In one embodiment, the ratio of peracetic acid to ethanol is from 1:500 to 1:10. In one embodiment, the ratio of peracetic acid to ethanol is from 1:100 to 1:10.

In one embodiment, the supercritical fluid comprises at least 95 wt % supercritical CO₂, 0.1-3 wt % ethanol, and 0.01-0.1 wt % peracetic acid. In one embodiment, the supercritical fluid comprises at least 98 wt % supercritical CO₂, at least 0.5-1.0 wt % ethanol, and at least 0.02-0.08 wt % peracetic acid. In one embodiment, the supercritical fluid comprises about 99 wt % supercritical CO₂, about 0.8 wt % ethanol, and at least 0.05 wt % peracetic acid.

In one embodiment, the removing step occurs at a temperature that is not greater than 40° C. In one embodiment, the removing step occurs at a temperature from 20° C. to 40° C. In one embodiment, the removing step occurs at a temperature from 25° C. to 38° C. In one embodiment, the removing step occurs at a temperature from 30° C. to 36° C. In one embodiment, the removing step occurs at a temperature from 33° C. to 34° C.

In one embodiment, the removing step comprises submerging the plant tissue in a non-polar solvent to remove a cuticle layer of the plant tissue, and perfusing the plant tissue with a fluid comprising a surfactant and bleach.

In one embodiment, the removing step comprises cannulation, then injection of chemical compounds (bleach, SDS, Triton X100, etc.) through the vasculature.

In one embodiment, the plant tissue is placed in a container with or without co-solvents and this container is placed into a pressure vessel for supercritical fluid treatment to allow the supercritical fluid to operate all over the leaf. In one embodiment, this treatment may take place at a temperature less than or equal 37° C. and a pressure between 70 and 210 atm. The plant tissue may comprise the combination of cannulation, injection of chemical compound(s) and supercritical fluid treatment.

In one embodiment, the method comprises rinsing the vascularized cellulose scaffold with water. In one embodiment, the method comprises treating the vascularized cellulose scaffold with collagen and fibronectin. In one embodiment, the method comprises seeding the vascularized cellulose scaffold with endothelial cells.

In one embodiment, the external stimulus comprises a pharmacological compound. In one embodiment, the external stimulus comprises ionizing radiation. In one embodiment, the external stimulus comprises a mechanical tear or puncture. In one embodiment, the external stimulus comprises a pesticide, a nerve agent, a corrosive liquid, or combinations thereof.

In one embodiment, a method of simulating a biological response of a multilayer cellular system comprises removing at least some DNA-containing material from a first vascular plant tissue to produce a first vascularized cellulose scaffold; removing at least some DNA-containing material from a second vascular plant tissue to produce a second vascularized cellulose scaffold; combining the first and second vascularized cellulose scaffolds to create a multilayered scaffold; seeding the multilayered scaffold with cultured biological cells; growing the cultured biological cells on the multilayer scaffold to produce a multilayered vascularized biological system; subjecting the multilayered vascularized biological system to an external stimulus; and measuring a response of the multilayered vascularized biological system. In one embodiment, the seeding step comprises seeding a first layer of the multilayered scaffold with cultured biological cells of a first cell type; and seeding a second layer of the multilayered scaffold with cultured biological cells of a second cell type. In one embodiment, the multilayered scaffold comprises a third vascularized cellulose scaffold. In one embodiment, the first cell type is keratinocytes and the second cell type is dermal fibroblasts. In one embodiment, the first layer of the multilayered scaffold is derived from a tomato leaf and the second layer is derived from spinach leaves. In one embodiment, the multilayered vascularized biological system is a living model of human skin.

In one embodiment, a method of producing a multilayered vascularized biological system comprises removing at least some DNA-containing material from a first vascular plant tissue to produce a first vascularized cellulose scaffold; removing at least some DNA-containing material from a second vascular plant tissue to produce a second vascularized cellulose scaffold; combining the first and second vascularized cellulose scaffolds to create a multilayer scaffold; seeding the multilayer scaffold with 3D structures of cultured biological cells; and growing the 3D structures of cultured biological cells on the multilayer scaffold to produce the multilayered vascularized biological system. In one embodiment, the seeding step comprises seeding the first vascularized cellulose scaffold with a first cell type of 3D structures of cultured biological cells; and seeding the second vascularized cellulose scaffold with a second cell type of 3D structures of cultured biological cells. In one embodiment, the multilayered vascularized biological system is a living model of human skin.

In one embodiment, a method of mimicking a vascular mammalian tissue includes storing a database of plant tissue characteristics for a plurality of plant tissues, analyzing a mammalian tissue of interest, selecting a first plant tissue from the database based on similarities between a first characteristic of the mammalian tissue and a corresponding first plant tissue characteristic stored in the database, producing a vascularized cellulose scaffold, wherein the producing includes removing at least some DNA-containing material from a sample of the first plant tissue, seeding the vascularized cellulose scaffold with mammalian cells corresponding to the mammalian tissue of interest, and growing the mammalian cells on the vascularized cellulose scaffold to produce an analogue vascular mammalian tissue. In one embodiment, the method my comprise selecting a second plant tissue from the database based on similarities between a second characteristic of the mammalian tissue and a corresponding second plant tissue characteristic stored in the database, wherein the producing step comprises: removing at least some DNA-containing material from a sample of the second plant tissue; and mechanically combining the first and second plant tissue samples to create a hybrid vascularized cellulose scaffold.

In one embodiment, the plant tissue characteristics stored in the database include a vascular structure parameter for each of the plurality of plant tissues, and a biomechanical structure parameter for each of the plurality of plant tissues. In one embodiment, the first characteristic is a vascular structure characteristic and the second characteristic is a biomechanical structure characteristic.

### BRIEF DESCRIPTION OF THE DRAWINGS

FIG. 1 shows a time lapse of leaf decellularization process.

FIG. 2A shows protein levels assayed before and after decellularization.

FIG. 2B shows the protein visualized by coomassie-stained gels.

FIG. 2C shows a quantification of DNA content analyzed via Epoch & Take 3 system (Biotek) after extraction of DNA using DNeasy blood & tissue kit.

FIG. 2D shows the extracted DNA visualized by agarose gel.

FIG. 2E is an Atomic Force Microscopy (AFM) image of a fresh leaf.

FIG. 2F is an AFM image of a decellularized leaf.

FIG. 2G is a comparison of Young's modulus between the fresh and decellularized leaves.

FIG. 3A is picture of a decellularized leaf repopulated with PC3 prostate cancer cells stained with DAPI, taken via differential interference contrast (left) and epifluorescence microscopy (right).

FIG. 3B is picture of a decellularized leaf repopulated with MCF7 breast cancer cells stained with DAPI, taken via differential interference contrast (left) and epifluorescence microscopy (right).

FIG. 3C is an AFM image of recellularized leaf.

FIG. 3D is a comparison of Young's modulus between fresh, decellularized and recellularized leaves.

FIG. 4A is a time lapse of the decellularization process over 5 days.

FIG. 4B is a comparison of DNA content in both fresh and decellularized leaves (n=3).

FIG. 4C is a comparison of protein content in both fresh and decellularized leaves quantified by micro BCA protein assay (n=3).

FIG. 5A is a visualization of the leaf scaffold and PC3 cells seeded on the leaf surface using Differential Interference Contrast (DIC) picture, DAPI-fluorescent picture (blue), F-actin-fluorescent picture (green). Scale bar=40 μm.

FIG. 5B shows AFM images showing surface structure of fresh, decellularized and recellularized leaves.

FIG. 5C is a comparison of the Young's modulus of fresh, decellularized and recellularized leaves represented as mean±SEM.

FIG. 5D is a visualization of β-catenin (red) by fluorescence microscopy.

FIG. 5E is fluorescence imaging of PC3 cells immunostained with anti α-tubulin monoclonal mouse IgG (red) 48 hours after seeding and counterstained with DAPI (blue) showing the presence of cells in different phase of mitosis: (1) prometaphase; (2) metaphase; and (3) late anaphase.

FIG. 5F shows proliferation of PC3 cells seeded on decellularized spinach leaf scaffold assessed by MTT assay over a period of 6 days.

FIG. 5G shows a co-culture of fibroblasts and PC3 cells.

FIG. 5H shows HUVECs that were injected into parsley stem and allowed to grow for 5 days.

FIG. 6A shows a volcano plot showing differentially expressed genes at 6 hours between 0 and 1 Gy-irradiated PC3 cells seeded on the leaf.

FIG. 6B shows representative nuclei of PC3 cells cultured on leaf with γ-H2AX and 53BP1 foci at 30 min, 6 and 24 hours after 2 Gy irradiation.

FIG. 6C shows individual dose response curves of CDKN1A, DDB2 and PCNA genes after 1 and 2 Gy irradiation of PC3 seeded on the leaf surface.

FIG. 6D shows cell survival curves of PC3 cells cultured on leaf and in cell culture flasks as assayed by colony-forming ability.

FIG. 7 is a schematic flow diagram of a method of mimicking a vascular mammalian tissue.

FIG. 8A shows an example of an Apium graveolens leaf decellularized via serial chemical treatment.

FIG. 8B shows an example of a Solanum Lycopersicum leaf decellularized via serial chemical treatment.

FIG. 8C shows an example of a Echinodorus grisebachii leaf decellularized via serial chemical treatment.

FIG. 8D shows an example of a Petroselinum crispum leaf decellularized via serial chemical treatment.

FIG. 9A shows 3D surface mapping of fresh spinach leaf by using gel-based photometric stereo profilometry.

FIG. 9B shows 3D surface mapping of decellularized spinach leaf by using gel-based photometric stereo profilometry.

FIG. 9C shows 3D surface mapping of recellularized spinach leaf by using gel-based photometric stereo profilometry.

FIG. 10A shows a comparison of the Young's modulus of fresh and decellularized aquatic plant, spinach, A. borealis and tomato leaves as measured by AFM.

FIG. 10B shows images of plant material before and after decellularization treatment.

FIG. 10C shows a comparison of DNA content in different fresh and decellularized plants (n=3).

FIG. 10D shows a comparison of protein content in different fresh and decellularized plants quantified by micro BCA protein assay (n=3).

FIG. 10E shows FM images showing surface of plant materials before and after decellularization treatment.

FIG. 11A shows immunofluorescence images of YAP/TAZ and nuclei (DAPI) in PC3 and SK-MEL-28 cells seeded on coverslip and spinach scaffold for 3 days.

FIG. 11B shows quantitative RT-PCR analysis in PC3 and SK-MEL-28 cells to measure YAP and TAZ mRNA levels.

FIG. 11C shows immunoblotting of YAP and TAZ in PC3 and SK-MEL-28 cells seeded on TCPS and spinach scaffolds.

FIG. 11D Quantitative RT-PCR for YAP/TAZ target genes (CTGF and ANKRD1) in PC3 and SK-MEL-28 cells.

FIG. 11E shows how functionalization increases the stiffness of a decellularized scaffold. Comparison of the Young's modulus of decellularized (D) and functionalized (C+F) spinach leaf with collagen and fibronectin as measured by AFM. The graph shows the mean±SEM (n=3, *p<0.05; Student t-test).

FIG. 11F shows the original uncropped blot of FIG. 11C. Red rectangles correspond to the cropped sections shown in FIG. 11C.

FIG. 12A shows representative SEM scanning images of PC3 and SK-MEL-28 cells cultured on stiff substrate (coverslip) or spinach leaf scaffold for 3 days.

FIG. 12B is a histogram showing the changes of cell spreading area on stiff and leaf substrates and represented as mean±SEM (n=3, *p<0.05; Mann-Whitney test).

FIG. 12C shows immunofluorescence images of F-actin and nuclei (DAPI) in PC3 and SK-MEL-28 cells seeded on stiff substrate (coverslip) and spinach scaffold. Scale bars=15 μm.

FIG. 13A shows immunofluorescence images of Ki-67 and nuclei (DAPI) in PC3 and SK-MEL-28 cells seeded on stiff substrate (coverslip) and spinach scaffold.

FIG. 13B is a histogram showing the percentage of Ki-67-positive cells and represented as mean±SEM (n=3, *p<0.05; Mann-Whitney test).

FIG. 13C shows cell proliferation of PC3 and SK-MEL-28 cells seeded for 7 days on stiff (TCPS) and spinach leaf substrate measured by modified MTT.

FIG. 14A shows immunofluorescence images of MITF and nuclei (DAPI) in SK-MEL-28 cells seeded on stiff substrate (coverslip) and spinach scaffold.

FIG. 14B is histogram showing the percentage of MITF-positive cells and represented as mean±SEM (n=3, *p<0.05; Student t-test).

FIG. 14C shows a quantitative RT-PCR analysis in SK-MEL-28 cells to measure MITF and there MITF target genes (SOX10, MLANA and TYR) mRNA levels.

FIG. 14D shows the effect of WFA treatment on SK-MEL-28 seeded on decellularized spinach leaves or TCPS assessed by modified MTT.

FIG. 15A is a graph showing differentially expressed genes at 6 hours between PC3 cells seeded on the leaf and TCPS.

FIG. 15B is a graph showing genes that are differentially expressed between PC3 cells seeded on the leaf and TCPS at 6 hours after 2 Gy-irradiation.

FIG. 15C is a representative nuclei of PC3 cells cultured on leaf with γ-H2AX foci at 1, 6 and 24 hours after 2 Gy irradiation (n=3).

FIG. 15D shows histograms showing the ratio of number of 2 Gy-irradiated γH2AX foci normalized to sham-irradiated samples, in PC3 cells seeded on spinach leaf or stiff substrate (coverslip).

FIG. 15E shows cell survival curves of PC3 cells cultured on leaf and in TCPS as assayed by colony-forming ability.

FIG. 16 shows a comparison of the Young's modulus of different decellularized plant scaffolds with TCPS and coverslips used in standard cell culture.

FIG. 17A shows immunofluorescence images of PC3 nuclei (DAPI) seeded on spinach scaffold (brightfield).

FIG. 17B shows 3D surface mapping of fresh, decellularized and recellularized spinach leaf by using gel-based photometric stereo profilometry.

FIG. 17C is a visualization of β-catenin (red) by fluorescence microscopy. Nuclei have been counterstained with DAPI (blue).

FIG. 17D shows immunofluorescence images of α-tubulin and nuclei (DAPI) of PC3 cells seeded on spinach scaffold showing the presence of cells in different phase of mitosis such as prometaphase (1), metaphase (2) and late anaphase (3).

FIG. 18 is a cutaway showing natural leaf architecture.

FIG. 19A shows a spinach leaf treated with sfCO₂ and 75% EtOH.

FIG. 19B shows a spinach leaf treated with sfCO₂ and 5% SDS in 75% EtOH.

FIG. 19C shows a spinach leaf treated with sfCO₂ and 10% Bleach in 75% EtOH.

FIG. 19D shows a spinach leaf treated with sfCO₂ and PAA 1% in 75% EtOH.

FIG. 19E shows a spinach leaf treated with sfCO₂ and PAA 2% in 75% EtOH.

FIG. 19F shows a spinach leaf treated with sfCO₂ and PAA 3% in 75% EtOH.

FIG. 20A shows images of plant material before and after decellularization treatment.

FIG. 20B shows a comparison of DNA content in different fresh and decellularized plants.

FIG. 20C shows a comparison of the YM of decellularized plants.

FIG. 20D shows a representation of the decellularized scaffolds' YM with the published stiffness values of different human tissue.

FIG. 21A shows a cross-section of one embodiment of a multilayered vascularized biological system with (1) stiff spinach leaves mimicking the stratum corneum; (2) tomato leaves seeded with keratinocytes mimicking the lower layers of epidermis; (3) soft spinach leaves seeded with dermal fibroblasts mimicking the dermis and (4) soft layer of A. borealis mimicking the subcutaneous layer, wherein the multilayered vascularized biological system has been stained with Hematoxylin/Eosin.

FIG. 21B shows a cross-section of the multilayered vascularized biological system stained with DAPI instead of Hematoxylin/Eosin.

FIG. 22 shows Fibroblasts and keratinocytes seeded on decellularized plant scaffolds and stained for F-actin (green) and β-catenin (green).

FIG. 23 is a Live-Dead assay showing fibroblast cells on spinach scaffold after 2 and 4 weeks of culture. Green/Red staining show Alive/Dead cells.

FIG. 24A shows the vasculature of decellularized spinach leaf enhanced with dyed water injection

FIG. 24B shows HUVECs cells seeded on spinach scaffold and stained for F-actin (green) and VE-cadherin (red)

FIG. 24C shows HUVECs seeded in spinach vasculature and stained for F-actin (green), nuclei are counterstained with DAPI (blue).

FIG. 25A shows immunofluorescence (IF) images of YAP/TAZ (red) and nuclei (DAPI).

FIG. 25B is a graph indicating the percentage of cells with nuclear YAP/TAZ.

FIG. 25C shows data normalized to expression on stiff substrate.

FIG. 25D shows the expression level compared to stiff substrate. qRT-PCR for YAP/TAZ target genes.

FIG. 25E shows data normalized to expression on stiff substrate.

FIG. 26A shows immunofluorescence images of organoids over 6 days.

FIG. 26B shows cell viability of DMSO-treated organoids irradiated at day 1.

FIG. 26C shows cell viability of everolimus-treated organoids irradiated at day 1.

FIG. 27A shows Glioblastoma (Glast) biomarker mRNA level measured by qRT-PCR from DMSO- and everolimus-treated organoids at day 10 after irradiation.

FIG. 27B shows SOX2 biomarker mRNA level measured by qRT-PCR from DMSO- and everolimus-treated organoids at day 10 after irradiation.

FIG. 27C shows Nestin biomarker mRNA level measured by qRT-PCR from DMSO- and everolimus-treated organoids at day 10 after irradiation.

FIG. 28A shows graphs of volume growth of sham- and 4 Gy-irradiated spheroids over 2 days after AP treatment.

FIG. 28B shows graphs of volume growth of sham- and 4 Gy-irradiated spheroids over 2 days after EA treatment.

FIG. 29 is a schematic diagram of the top view of the experiment layout used for the sample irradiations. The red arrows indict the cone of neutrons incident on the sample stack. The diagram is not to scale.

FIG. 30 is a plot of the differential cross section for the 2H(d,n)3He neutron source reaction as a function of the laboratory angle of the neutrons emitted relative to the deuteron beam axis shown in FIGS. 20A-20D. The data are from, Journal Nucl. Sci. & Eng. 67, 190 (1978). The shaded region indicates the angular coverage of the samples positioned as shown in FIGS. 20A-20D during irradiation.

FIG. 31A shows a spinach leaf before (fresh) and after sfCO2 decellularization.

FIG. 31B shows DNA content of chemically and sfCO2 decellularized spinach leaves compared to fresh leaves (n=3, *p<0.05 vs fresh by student t-test).

FIG. 31C shows protein content of chemically and sfCO2 decellularized spinach leaves compared to fresh leaves (n=3, *p<0.05 vs fresh by student t-test).

FIG. 32 shows the effect of WFA treatment on cells seeded on different substrates assessed by MTT.

FIG. 33 RFP-transfected dermal fibroblasts spheroids have been exposed to different concentrations of everolimus 24 h before 4 Gy-irradiation and their growth monitored for 8 days.

FIG. 34 Decellularized leaf under stretch deformation.

FIG. 35 Comparison of the YM of decellularized plants.

FIG. 36 shows mass transport through the multilayered vascularized biological system. The upper layer mimicking stratum corneium was soaked in ponceau red solution for 10 minutes then transferred to the skin-on-leaf system. After 2 hours incubation the system was disassembled and the stain of each layer was visually compared.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

As used herein, “biological system” is used broadly herein and refers to an in vitro biological unit of an organism that is useful in a variety of applications, including modelling and testing systems. A biological system may correspond to an organ, or a portion thereof, including cellular components thereof. For example, a biological system may be a lung, liver, intestine, heart, brain, etc., or cells thereof, including cells associated with blood vessels (e.g., endothelial cells, smooth muscle cells, and the like), neuronal cells, etc. A biological system may be a collection of one or more organs, tissue and/or biological fluid (e.g., blood, plasma, interstitial fluid, etc.) organized to perform one or more biological functions. For example, a biological system may be the respiratory system, digestive system, cardiovascular system, endocrine system, etc. A biological system may include a biological abnormality such as cancer. Accordingly, a “vascularized” biological system refers to a network of conduits within the biological system that is capable, at least in part, of transporting required oxygen and nutrients to the biological cells and removing waste product from the biological cell. The exchange may be similar to how exchange occurs in blood vessels, such as by diffusion. The systems are, of course, compatible with bulk media control, where the cultured cells are immersed in a cell culture fluid that can be replaced or supplemented as desired.

“Three dimensional structures” or “3D structures” of cultured biological cells refers to a collection of cultured cells joined together via cell adhesion wherein the collection of cells is many cells across in all three spatial dimensions. A 3D structure of cultured biological cells may be at least 10 cells across in all three spatial dimensions. A 3D structure of cultured biological cells may be at least 100 cells across in all three spatial dimensions. Accordingly, as used herein a monolayer of cultured cells is not considered a 3D structure of cultured biological cells. In some embodiments, a 3D structure of cultured biological cells is a tissue, an organ, or a cancer mass. A 3D structure of cultured biological cells can include any eukaryotic cells that are able to self-organize in order to form a 3D structure that influences the spatial organization of the cell surface receptors engaged in interactions with surrounding cells, and may also induce physical constraints on the cells. These spatial and physical aspects in 3D cellular structures affect the signal transduction from the outside to the inside of cells, and ultimately influence gene expression and cellular behavior in a manner closer to that of the in vivo environment than a standard cellular monolayer culture. The systems and methods provided herein are compatible with a range of biological cell sources, including cells that have not been cultured, so long as the seeded cells are capable of subsequent cell culture after being introduced to the decellularized plant tissue vascular scaffold.

In the context of “mimicking a vascular mammalian tissue”, “mimicking” refers to in vitro reproduction or simulation of one or more functions of a mammalian tissue. Mimicking a vascular mammalian tissue may include in vitro simulation or reproduction of the vascular structure of the mammalian tissue. Mimicking may refer to statistical matching between the in vitro 3-D system provided herein and a corresponding biological tissue that is being modelled. For example, the matching may correspond to matching of the vasculature, geometry, shape, and/or cell type.

Depending on the tissue being modeled, the constituents of the in vitro system may include one or more of: mammalian cells, a plurality of mammalian cell types, extracellular tissue, vessels, bacterial cells, cell culture media, perfusion media, and the like.

Devices and methods for simulating a physiological system of soft tissue are described. Features may include mimicking a vasculature system, for determining dynamic responses when exposed to external stimuli, for example, a pharmacological compound, ionizing radiations, toxic chemicals such as pesticides, nerve agents, corrosive liquids and so on. In a specific embodiment, the system may act as a 3D biomimetic phantom using several biological materials assembled into a scaffolding organ-like structure of tissue equivalent for determining the effects of radiation doses. The system architecture allows for a dynamic interrogation mimicking blood circulation, for example. The system may also have different controllable and localized mechanical properties tuning the materials from soft to hard regions that can influence biological responses.

In one embodiment, a method comprises selecting a three-dimensional biological system from a first species, and preferably from a plant, whose three-dimensional structure is arranged into tubular composite sub-structures (e.g. fibers) embedded into matrix materials to form a patterned network that can be populated with other biological components from a second species to form an environment suitable for self-monitoring multiple responses when said biological system is induced by a stimuli, for example a chemical, biological or radiation exposure.

The method may comprise the steps of: (1) connecting the tubular sub-structures to a fluid source by inserting a fluidic device (e.g. capillary tube, needle) and placing a thermal or chemical shrink materials encapsulation for creating a tight environment. The fluid source may be comprised of liquid solutions with chemicals from the family of detergent, surfactant, solvent, acid, base or mixture thereof, sub/supercritical fluids (e.g. CO2), biochemicals from the family of biological fluids (e.g. blood, saliva, urine, sweat, enzymes, cells, nucleic acids, proteins, metabolites, vitamins, effusions and the like.) or gases (e.g. air, CO2 and the like). The fluid may be delivered by gravity-driven motion, thermal or pressure activation by mechanical, electromechanical, or magnetic transducers moving fluid through said biological component to modify its composition and preferably removing cells, proteins, nucleic acids and other genetic materials while mostly preserving the scaffold structure and morphology of said component; (2) selecting cells from a second species and preparing culture conditions and components to be transferred onto said biological system to modify its composition by performing cell culture growth and progression through said scaffold to build a biological environment which can be sustained to build new constructs (e.g. tissue, organs and the like) that can enter a communication with other biological or molecular compounds circulated through said tubular sub-structure network, or by other environmental exposure and/or induction.

### Example 1: Leaf Decellularization and Subsequent Recelluarization with Cancer Cells

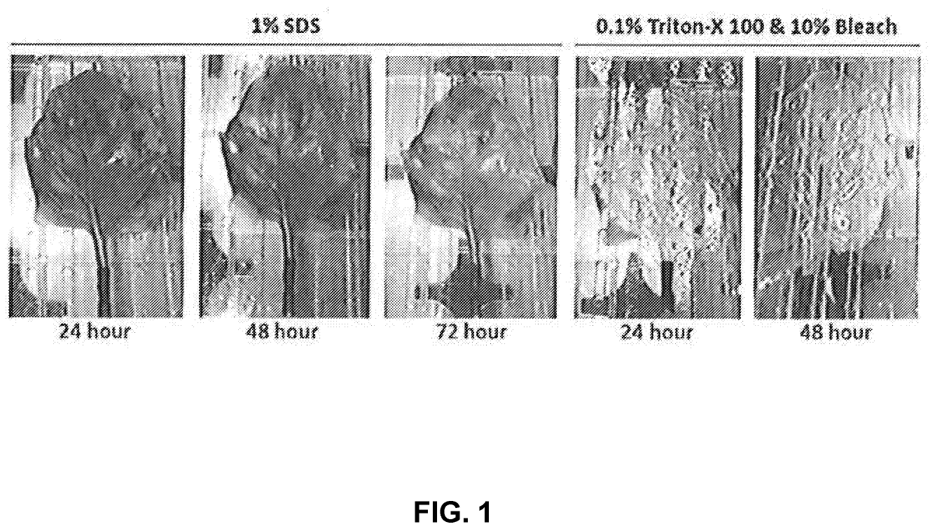

A new 3D culture model was established by using a spinach leaf scaffold. Spinach leaves are perfused with serial chemical treatments to remove all plant cells. Once decellularized, a predominantly cellulose, human cell compatible scaffolding remains which can be used to attach lung cells and allow them to grow in a spatially relevant environment. A time lapse of the method is shown in FIG. 1. Spinach leaves are first cannulated with a 26 gauge needle and surrounded with heat shrink tubing. Leaves are washed in hexanes and rinsed with PBS to remove the cuticle. Cannulated leaves are then perfused with a 1% SDS solution for 3 days followed by a treatment with 10% sodium chloride bleach and 0.1% Triton X-100 for 2 days at room temperature. The decellularized leaf is then washed for 2 days using deionized water.

As shown in FIG. 1, leaves show dark green opaque color prior to decellularization at day 0. Rapidly, the leaf becomes slightly translucent. At the end of treatment, the leaf loses all its color and becomes completely white and transparent. To assess the decellularization efficiency, protein and DNA content was measured before and after treatment as well as Young's modulus by using atomic force microscopy (AFM) to determine the leaf's rigidity.

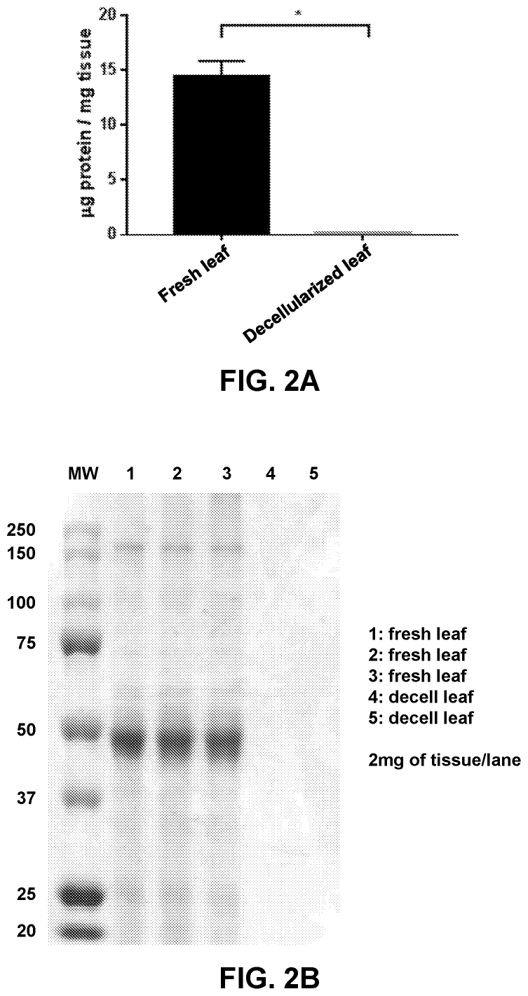

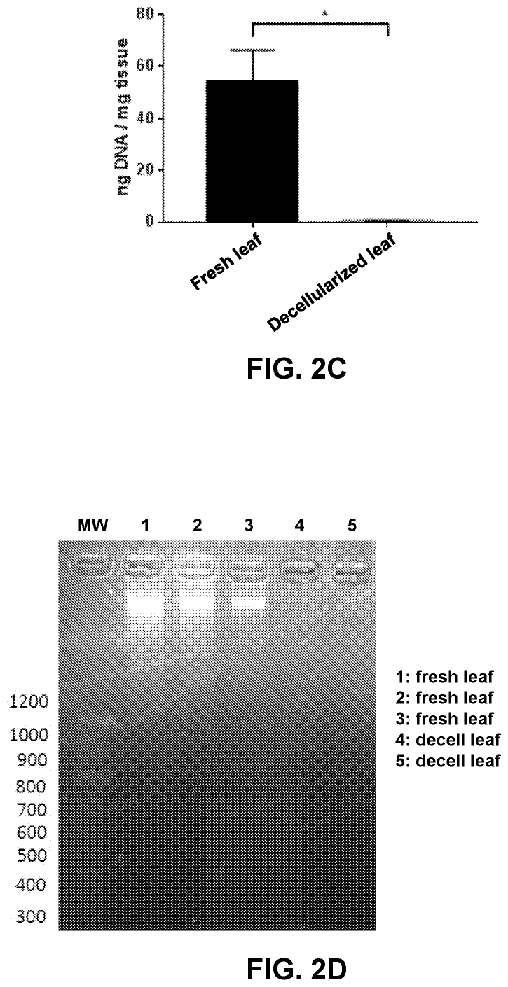

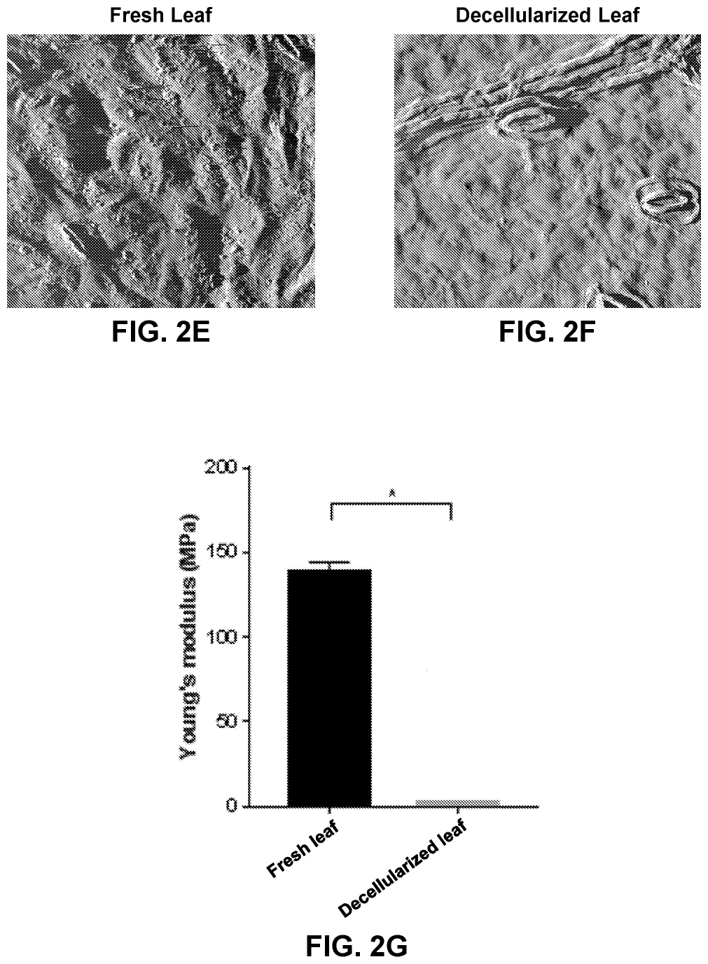

FIGS. 2A-2G shows an assessment of decellularization efficiency. FIG. 2A shows protein levels assayed before and after decellularization. FIG. 2B shows the protein visualized by coomassie-stained gels. DNA content was quantified through Epoch & Take 3 system (Biotek) after extraction of DNA using DNeasy blood & tissue kit (FIG. 2C) and visualized by agarose gel (FIG. 2D). Image of a fresh (FIG. 2E) and decellularized leaf (FIG. 2F) by using Atomic Force Microscopy (AFM). Comparison of Young's modulus between fresh and decellularized leaves measured by AFM (FIG. 2G).

Thus, decellularized leaves contained significantly less protein (1.2 vs. 14.4 μg protein/mg, s) and significantly less DNA (1.3 vs. 57.2 ng DNNmg, FIG. 2CD) demonstrating that the chemical treatment eliminated nearly all of the plant protein and DNA. Thus, vegetal cells were efficiently removed via the treatment. Additionally, AFM pictures show a different structure between fresh and decellularized leaves where stomata and veins appear clearly in the decellularized leaf suggesting plant cells were completely removed (FIGS. 2E-2F). Fresh leaves have a higher average Young's modulus (132 MPa) than decellularized leaves (12 MPa) demonstrating that rigidity of decellularized leaves significantly decreased due to cell removal.

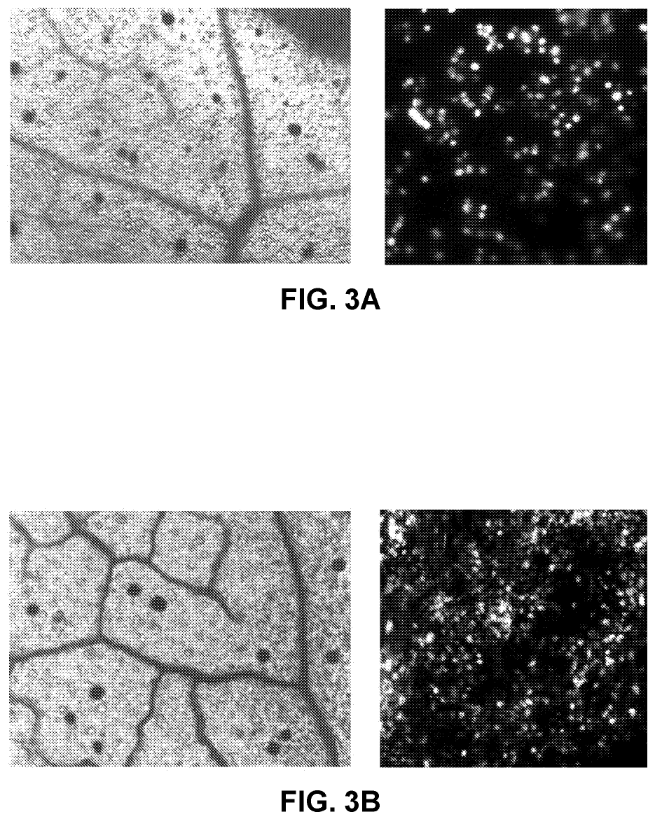

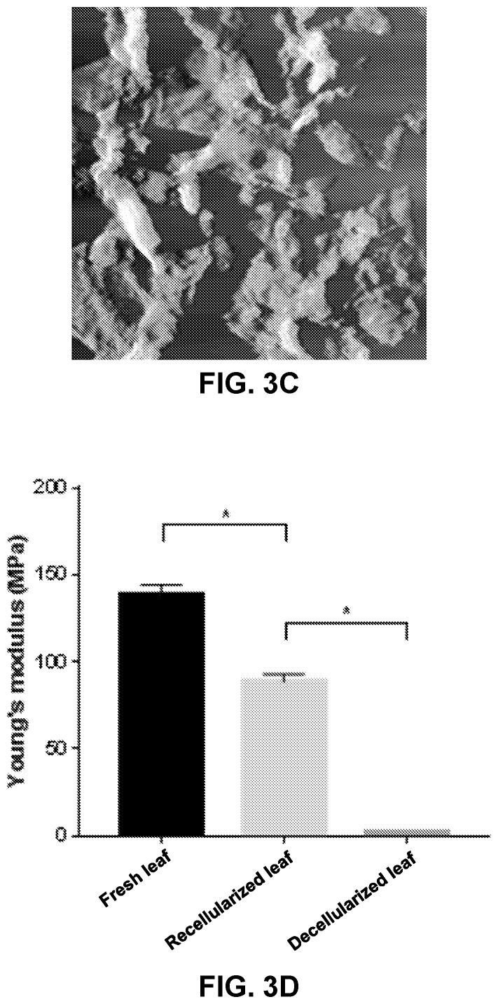

Subsequent to decellularization, prostate cancer cell line (PC3) and breast cancer cell line (MCF7) were seeded on the external surface of decellularized leaves after 24 hr. treatment with fibronectin, EDTA or cell culture medium. Recellularized leaves with PC3 prostate cancer cells can be seen in FIG. 3A and MCF7 breast cancer cells can be seen in FIG. 3B. Pictures have been taken through an optical microscope with fluorescence microscopy where cells have been treated with DAPI staining. Image of recellularized leaf by using Atomic Force Microscopy (AFM) is shown in FIG. 3C. Comparison of Young's modulus between fresh, decellularized and recellularized leaves measured by AFM is shown in FIG. 3D.

Unexpectedly, even without specific treatment, human cells were able to attach to the decellularized leaves, as shown in FIGS. 3A-3B. DAPI staining showed that the cells adhered at different depths within the scaffold thus presenting a true 3D structure. As show in FIGS. 3C-3D, measurement of Young's modulus by AFM three days after cell seeding showed that recellularized leaves are more rigid than decellularized leaves even if they do not show same rigidity as fresh leaves (98 MPa vs. 12 MPa vs. 132 MPa, respectively) confirming that human cells are firmly attached to the scaffold.

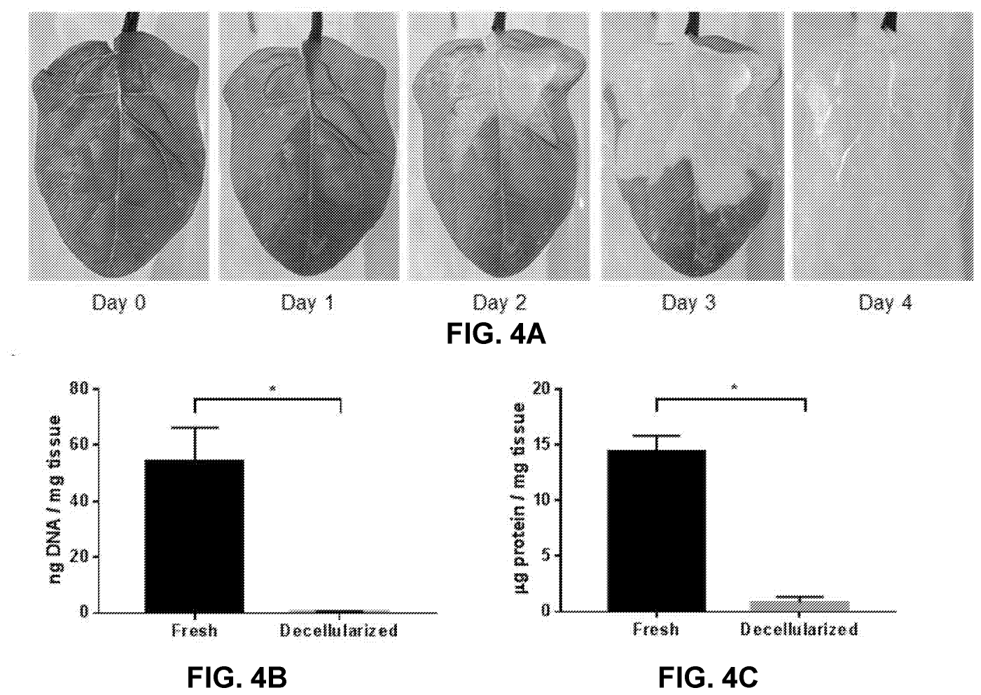

Viability and proliferation of PC3 cancer cells seeded on the leaf was assessed by using a tetrazolium (MTT) colorimetric assay. Absorbance increases along the 6 days of cell culture suggesting that cells are alive and able to proliferative on the spinach scaffold, shown in FIG. 4A (Time Lapse of decellularization process over 5 days. The first two days leaf is treated with 1% SDS solution and then treatment is switched to a 10% sodium chlorite/0.1% Triton-X 100 solution until spinach leaf becomes completely translucent). FIG. 4B is a comparison of DNA content in both fresh and decellularized leaves (n=3). FIG. 4C is a comparison of protein content in both fresh and decellularized leaves quantified by micro BCA protein assay (n=3).

Second, to evaluate if cells are also able to respond to external stimuli, cells seeded on the leaf were irradiated and a y-H2AX assay was performed by using fluorescence microscopy. Results showed that fluorescence level in cell nuclei increases for one hour post-irradiation compared to the non-irradiated control, shown in FIG. 4B. This demonstrates that y-H2AX is recruited for radiation-induced DNA damage repair and therefore cellular response to radiation is active in cells seeded on leaves. Finally, gene expression level of eight well known radiation response genes were compared before and after 5 Gy irradiation in MCF7 cells seeded either on a leaf or in a standard 20 cell culture dish. Unexpectedly, results showed that gene expression after irradiation is different between cells cultured onto the spinach leaf versus standard cell culture, shown in FIG. 4C. Without wishing to be bound by theory, this may be explained by the structure of the leaf allowing cells to grow in three-dimensional configuration under relevant biological mechanical stress. Thus, viable cancer cells, responsive to stimuli, can populate the leaf scaffold.

### Example 2: Vascularized Cellulose Scaffolds to Mimic TME and Provide a New In Vitro Model to Investigate Human Prostate Cancer Tumor Response to IR

The use of decellularized plant leaves as a vascularized scaffold to be repopulated by human cells was assessed. Results show that decellularization of spinach leaves can provide a robust cellulose scaffold with mechanical properties allowing for attachment of prostate cancer cells, stroma cells (fibroblasts) and endothelial cells. Cancer cells may survive and proliferate for up to one week. Unexpectedly, they also respond to radiation-induced DNA damage as shown by expression level of DNA damage-involved genes and proteins. Moreover, clonogenic survival shows that cancer cells radiosensitivity is not modified by the attachment on the plant scaffold. Thus, in one embodiment, plant-based scaffolds as disclosed herein may be used to mimic TME and study radiation response

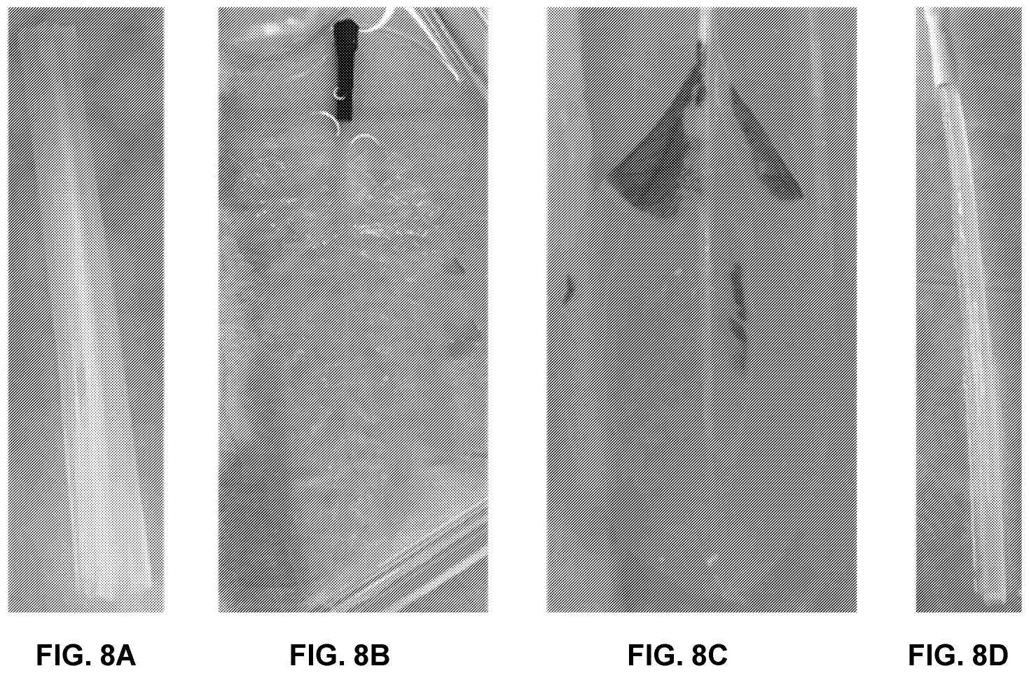

Baby spinach leaves were cannulated through their petiole, with a 26-gauge needle wrapped with a heat shrink tubing, and then washed three times in hexanes to remove cuticle. In a humidified environment, leaves were then perfused with 1% sodium dodecyl sulfate (SDS) solution for 2 days, followed by 2 days of perfusion with 0.1 TritonX-100, 10% sodium chlorite solution and finally rinsed for 2 days with deionized water. As soon as 24 hours after the start of treatment, the leaves began to lose chlorophyll to become finally fully translucent at Day 5 (FIG. 4A). To assess the efficiency of the decellularization process, protein and DNA content was then quantified. Decellularized leaves contained significantly less DNA (FIG. 4B) and protein (FIG. 4C) compared to fresh leaves suggesting that decellularization treatment was effective to remove all vegetal content. Following the same approach, we also managed to decellularize other types of plant, allowing us to create different scaffolds, which could be used for various tumor models according to their specific venation patterns, size, shape, and mechanical properties. See, e.g, FIGS. 8A-8D.

### Recellularization of Cellulose Scaffold

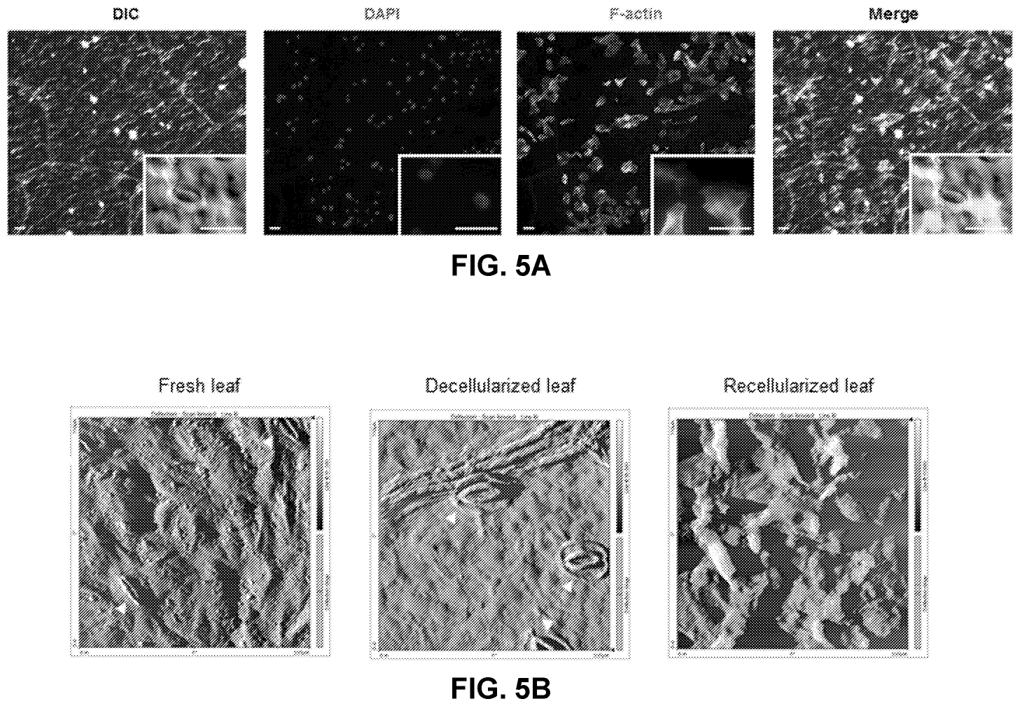

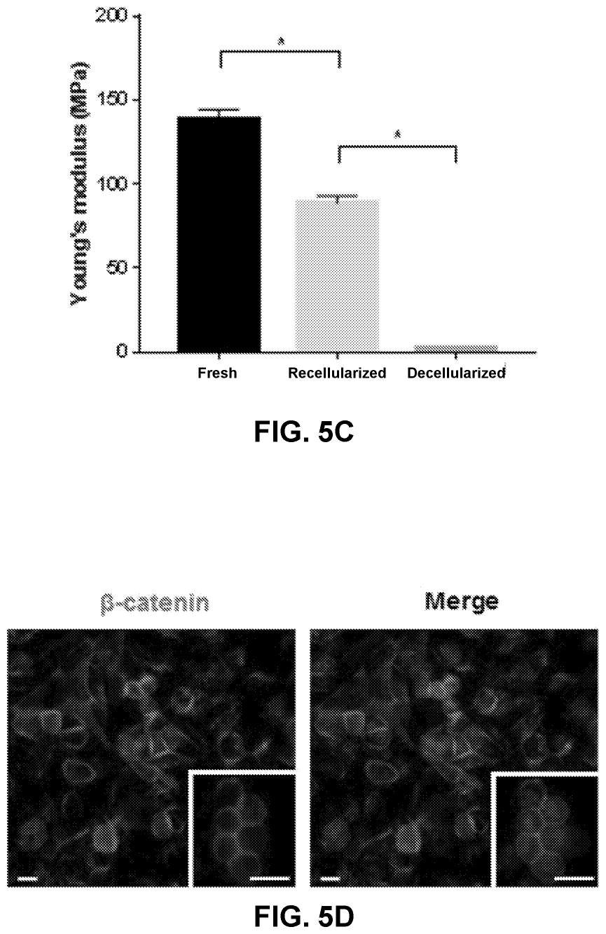

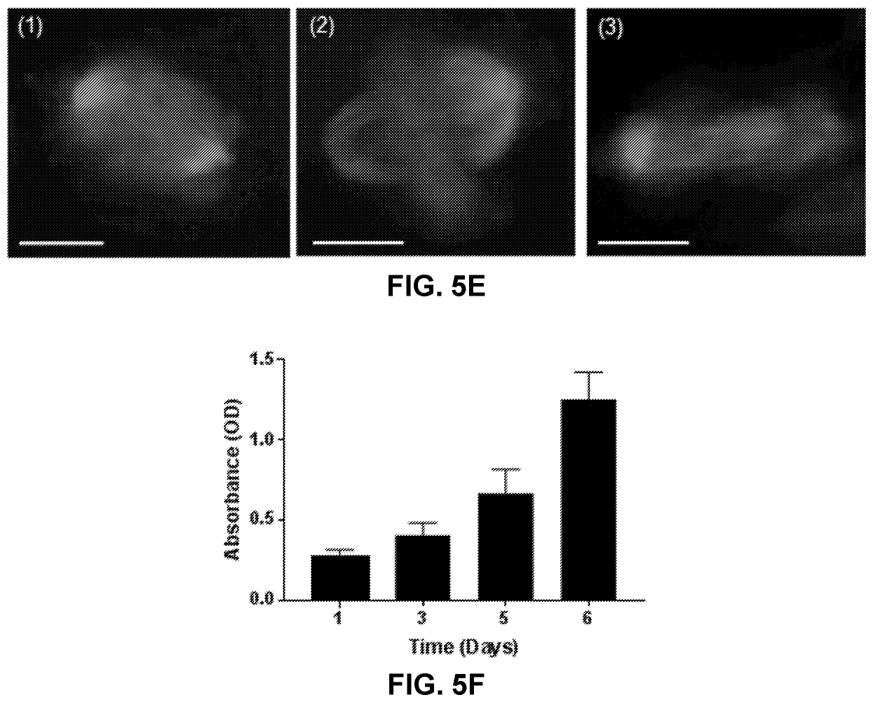

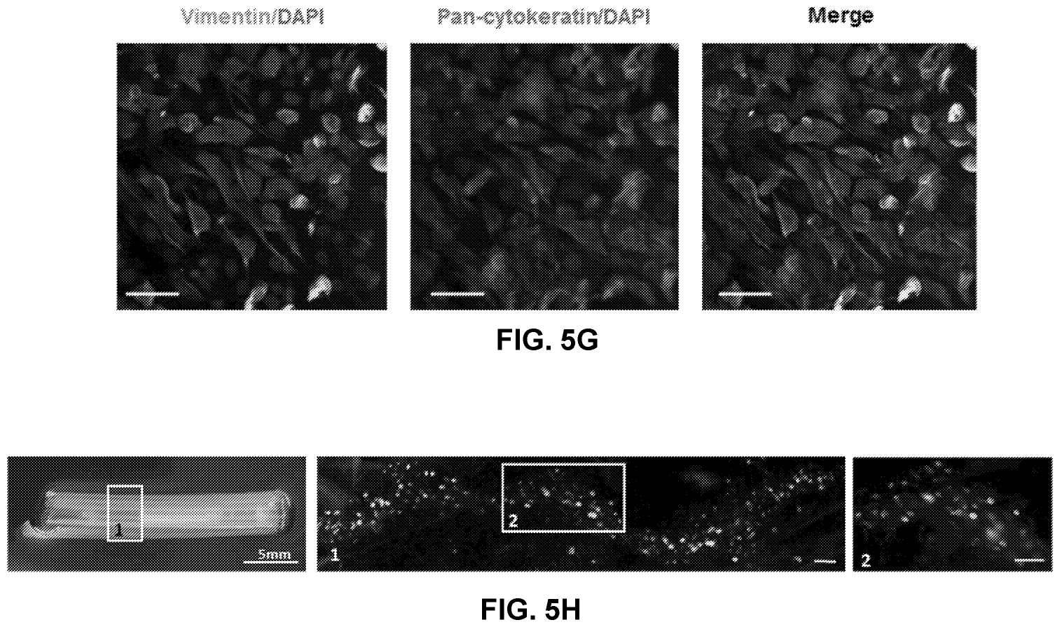

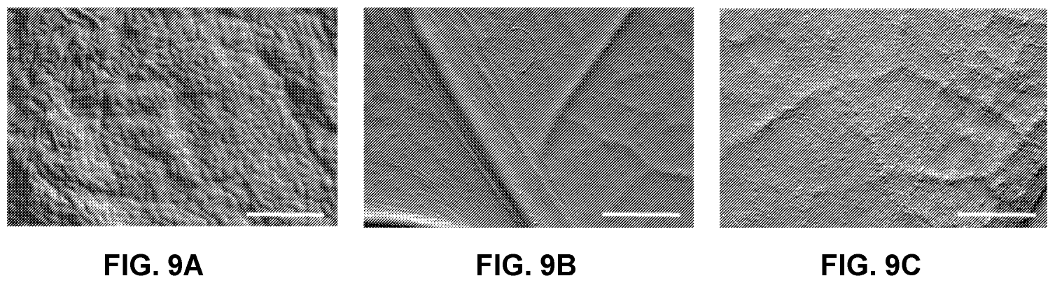

Adherence of human prostate cancer cells (PC3) on the decellularized scaffold was accessed. Leaf structures were first functionalized with a combination of collagen and fibronectin treatment in order to better mimic TME and presence of ECM. After 24 h incubation, fluorescence microscopy imaging shows that PC3 are present on the leaf surface are shown in FIG. 5A. (Visualization of the leaf scaffold and PC3 cells seeded on the leaf surface using Differential Interference Contrast (DIC) picture, DAPI-fluorescent picture (blue), F-actin-fluorescent picture (green). Scale bar=40 μm.) Long and well-formed actin fibers are visible showing that cells adhere and spread correctly on the functionalized cellulose scaffold. Atomic force microscopy (AFM)-based imaging and force mapping were then used to confirm cell attachment. First, images contouring leaf surface show that plant structures such as stoma and veins are more visible on the decellularized leaf compared to fresh leaf suggesting that vegetal cells are actually removed from the native sample and confirming the high efficiency of decellularization process is shown in FIG. 5B (AFM images showing surface structure of fresh, decellularized and recellularized leaves. Image size 110×110 μm. White arrows indicate stoma.) Second, the surface of recellularized leaf shows a distinct relief compared to the decellularized leaf suggesting that cells bound to the cellulose scaffold, and repopulated it. A larger scale optical 3D leaf topography and texture imaging using a GelSight tactile sensor¹¹ was performed. The photometric results showed identical results to AFM scans (i.e. apparent vasculature on decellularized leaf masked by presence of plant cells on fresh leaf) as shown in FIGS. 9A-9C.

Finally, measurement of Young's modulus by using force-distance curves-based AFM showed that the recellularized scaffold is more rigid than the decellularized scaffold (88.65±4.3 vs. 2.81±0.03 MPa) although it is still less robust than the native leaf (139.36±5.00 MPa) as shown in FIG. 5C (Comparison of the Young's modulus of fresh, decellularized and recellularized leaves represented as mean±SEM.) All together, these results demonstrate that PC3 cells may attach onto the functionalized scaffold.

It has been discovered that a tissue-like layer may be formed via the repopulated cellulose scaffold. Fluorescence imaging showed that cells on the scaffold may create cell-cell junctions, such as adherens junctions, when visualized by the expression of β-catenin displayed in FIG. 5D (visualization of β-catenin (red) by fluorescence microscopy. Nuclei have been counterstained with DAPI (blue). Scale bar=25 μm.) Accordingly, cells can develop as a fully differentiated and polarized tissue-like monolayer. Moreover, well-formed mitotic spindles are visible in PC3 cells showing different phases of mitosis and thus demonstrating that cells are alive and able to divide on the leaf scaffold as shown in FIG. 5E (PC3 cells were immunostained with anti α-tubulin monoclonal mouse IgG (red) 48 hours after seeding and counterstained with DAPI (blue). Fluorescence imaging showed the presence of cells in different phase of mitosis such as prometaphase (1), metaphase (2) and late anaphase (3) Scale bar=10 μm.) This has been confirmed using a cell proliferation assay showing that PC3 cells proliferate up to 6 days as shown in FIG. 5F (Proliferation of PC3 cells seeded on decellularized spinach leaf scaffold assessed by MTT assay over a period of 6 days. Data are represented as mean±SEM of 3 independent experiments with 4 replicates each.). Unexpectedly, a co-culture of epithelial and fibroblasts cells was developed, as shown on FIG. 5G (Co-culture of fibroblasts and PC3 cells. Cells were immunostained with anti-vimentin polyclonal rabbit IgG (green) and anti pan-cytokeratin monoclonal mouse IgG (red) and counterstained with DAPI (blue). Scale bar=50 μm.), where vimentin staining is mainly present in fibroblast cells while pan-cytokeratin staining was only present in PC3 cells.

It has also been discovered that human cells may be attached to the inner surfaces of the vasculature of the scaffold. Human umbilical vein endothelial cells (HUVECs) were injected into a parsley stem and allowed to proliferate for 5 days. Fluorescence microscopy showed that they were able to colonize and attach the inner part of the stem is shown in FIG. 5H (HUVECs were injected into parsley stem and allowed to grow for 5 days. Parsley stem was then immunostained, cut in half and imaged with fluorescence microscopy (F-actin, green; nucleus, DAPI). Scale bar=100 μm unless specified.).

Thus in one embodiment, the decellularized vascularized scaffold may be repopulated by the appropriate human cells to mimic a human tissue.

### Radiation Response of Cells Seeded on Cellulose Scaffold

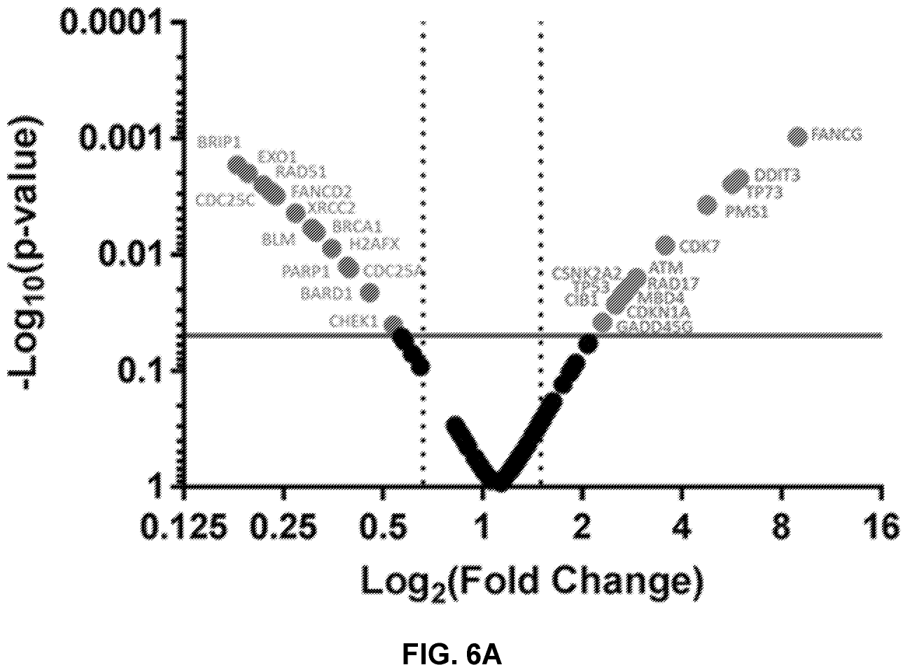

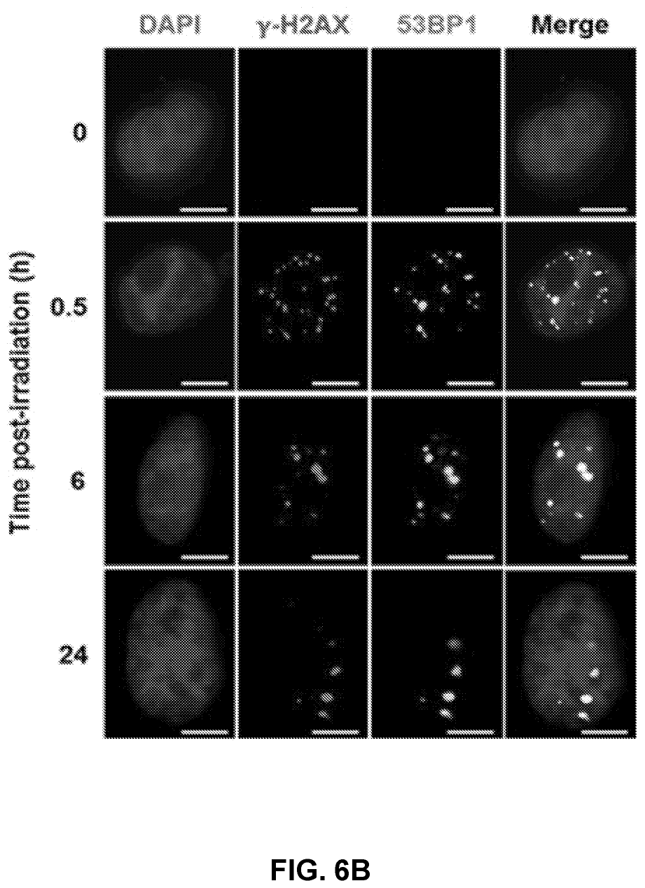

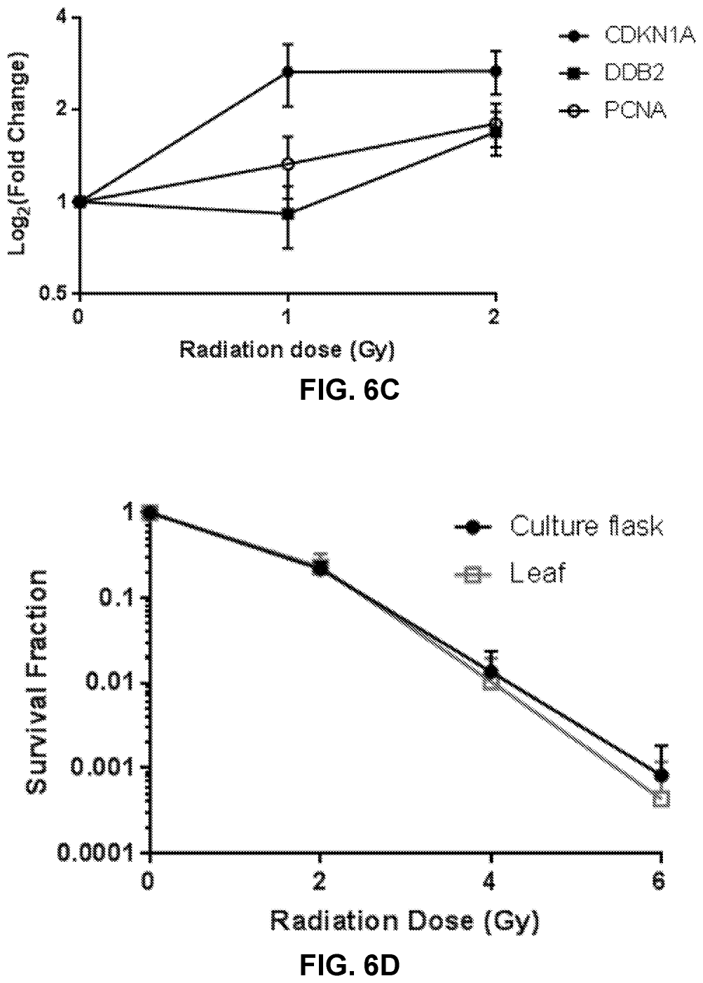

Response of the cells growing on the scaffold external IR was then assessed.. First, expression of 84 genes involved in DNA damage signaling pathways after 1 Gy irradiation was assessed. FIG. 6A is a volcano plot showing differentially expressed genes at 6 hours between 0 and 1 Gy-irradiated PC3 cells seeded on the leaf. A total of 85 genes values have been measured (including black, red and green dots). 26 genes have |log 2 expression change|>1.5 and p-value <0.05 (up-/downregulated genes, red/green dots respectively). Results showed that 13 genes were significantly upregulated (fold change >1.5) and 13 genes downregulated (fold change <0.66) suggesting that cells seeded on the leaf surface responded to radiation stress. To confirm, the kinetics of radiation-induced DNA double-strand breaks (DSBs) repair was evaluated by measuring foci formation of two markers of DSBs (γ-H2AX and 53BP1). The number of γ-H2AX and 53BP1 foci per nucleus was visualized in PC3 cells at 30 min, 6 and 24 hours after 2 Gy irradiation.

FIG. 6B shows representative nuclei of PC3 cells cultured on leaf with γ-H2AX and 53BP1 foci at 30 min, 6 and 24 hours after 2 Gy irradiation. Scale bar=8 μm. As shown in FIG. 6B, IR significantly increased the number of γ-H2AX and 53BP1 foci 30 minutes post-irradiation compared with sham-irradiated cells. From 6 hours, foci number decreased and came back to baseline level at 24 hours, confirming that cells were damaged by irradiation and were able to initiate and perform DNA damage repair. The dose dependency of the cellular response was investigated. FIG. 6C shows individual dose response curves of CDKN1A, DDB2 and PCNA genes after 1 and 2 Gy irradiation of PC3 seeded on the leaf surface. Data indicate mean±SEM. Expression of 3 well-established radiation dosimetry genes (CDKN1A, DDB2 and PCNA) was measured 6 hours after 1 and 2 Gy irradiation. Results showed increasing gene expression with increasing dose (FIG. 6C). Thus, it is believed that the cell response to IR is a function of the dose, and does not act as a dichotomous on/off response. Finally, a clonogenic assay was performed on PC3 cells that proliferated for three days either in culture flask or on a leaf in order to assess if the leaf scaffold could affect cell radiosensitivity. FIG. 6D shows cell survival curves of PC3 cells cultured on leaf and in cell culture flasks as assayed by colony-forming ability. Colonies with more than 50 cells were scored. Data represent mean±SEM of three independent experiments. As shown in FIG. 6D, the survival fraction of cells cultured on the leaf is the same as the one of cells cultured in flaks, suggesting that radiosensitivity of PC3 cells is similar between the two cell culture conditions.

Thus in one embodiment, vascularized cellulose scaffolds disclosed herein are used to mimic TME and provide a new in vitro model to investigate tumor response to IR.

In one embodiment, vegetal structures may be decellularized by chemical treatment and their resulting cellulose scaffold may be repopulated with human cells both on the surface and in the inner plant vasculature. In one aspect, the seeded human cells are able to form cell junctions and proliferate, and are metabolically active and functional, as demonstrated by their response to external ionizing radiation. In one embodiment, diversity in leaf structures offer a large choice of different scaffolds with different properties to possibly match some of the human tumor phenotypes. In one embodiment, the leaf nerves provide a scaffold to mimic different types of vasculature architecture. In one embodiment, the TME may be recapitulated via co-culture of blood vessels-like structures in close proximity with stroma and cancer cells seeded on the surface, as present in in vivo tumor. Current bioengineering techniques, such as microfabricated devices and 3D printing, are unable to create patent perfusion vessels and functional microvasculature inferior to a few microns in diameter and therefore do not reproduce the 100-200 μm oxygen diffusion limit normally encountered within human tissues.

Second, malignant transformation is also accompanied by specific changes in cellular and extra-cellular mechanical properties which regulate a wide range of cellular responses critical to tumorigenesis. Accordingly, in one embodiment, biophysical characteristics including matrix stiffness, pore size, viscoelasticity, crosslinking proteins and density, fiber network configuration, and/or cancer cell stiffness may be simulated via proper selection of plant species. Each plant species being unique and displaying different strength and hardness of leaves, cellulose scaffolds provide matrix with different stiffness, elasticity and cell adhesion to better recapitulate the mechanical properties encountered in different types of tumors or at different stages of disease progression. Thus, plant-based scaffolds and methods disclosed herein may be used to mimic complex cellular and molecular interactions, such as response to IR.

### Decellularization of Plant Tissues

To initiate decellularization, a spinach leaf was cannulated through the petiole (base of the stem) with a 26-gauge needle and secured with heat shrink tape tubing. The wax cuticle protecting the leaf was removed with 3 cycles of alternating washes with hexanes and phosphate-buffered saline (PBS). The prepared leaves were connected to the gravity bags filled up with the different solutions. Decellularization by chemical treatment started with perfusion of a solution of 1% sodium dodecyl sulfate (SDS) in deionized (DI) water for 2 days, followed by a solution of 10% sodium chlorite and 0.1% Triton-X 100 in DI water for 2 days. The leaves were then flushed with DI water for an additional 48 hours. Leaves were stored in DI water at 4 degrees Celsius (° C.) until ready to use.

### DNA/Protein Quantification

Leaves were first placed in centrifuge tubes and then were disrupted after freezing in liquid nitrogen by using a pestle. Resulting powder were transferred to a microcentrifuge tube for DNA and protein quantification. DNA content of fresh and decellularized leaves was extracted using PureLink Genomic DNA Mini Kit (Invitrogen) following manufacturer recommendation, while proteins were extracted by radioimmunoprecipitation assay (RIPA) (Pierce RIPA buffer, ThermoFisher Scientific). DNA content was quantified by reading absorbance at 260 nm and protein content was quantified using microBCA protein assay kit (ThermoFisher Scientific). Both absorbance were read by using Epoch microplate spectrophotometer (BioTek Instruments).

### Human Cell Culture and Seeding

PC3 and fibroblasts were cultured in RPMI 1640 medium containing 10% fetal bovine serum (FBS) and 1% Penicillin/Streptomycin (P/S) and HUVECs in Endothelial Cell Growth Medium (Cell Applications, Inc., Cat. No. 211-500). Before recellularization, leaf and stem scaffolds were first sterilized using UV Stratalinker 2400 (Stratagene) for 30 minutes. For PC3 and fibroblasts, leaf structures were functionalized with collagen and fibronectin proteins. Briefly, scaffolds were incubated in 50 ug/ml of collagen I (A1048301, Thermo Scientific) in 20 mM acetic acid solution for 4 hours, followed by two washes in PBS and a final wash in complete medium. Leaves were then incubated in 10 ug/ml fibronectin (F0895, Sigma-Aldrich) for 24 hours followed by three washes in complete medium. Finally, treated leaves were cut into small pieces and fit to the bottom of untreated multiple well plates. Inserts were placed in the well, on the top of the leaf, to contain the cells and facilitate cellular attachment. For HUVECs, one million cells were concentrated in 100 uL Endothelial Cell Growth Medium and mixed with equal ratios of Geltrex gel (Geltrex™ LDEV-Free Reduced Growth Factor Basement Membrane Matrix, Cat. No. A1413202, ThermoFisher Scientific) before seeding inside of the decellularized parsley stem. Geltrex coating concentration was determined by calculating the surface area inside of the parsley stem. Once seeded, cells were incubated for 30 minutes at 37° C. to allow for substrate formation followed by an additional 2 hours before perfusing fresh medium to promote cell attachment. Cells were allowed to attach for different times, depending on the assay, at 37° C. with 5% CO₂ atmosphere.

### Atomic Force Microscopy (AFM)-Based Imaging and Force Mapping

The AFM instrument used was Nanosurf LensAFM (Nanosurf, Switzerland) coupled with an upright Nikon Eclipse E800 optical microscope (Nikon, Japan). The cantilevers (Veeco) were 405-495 μm long, 45-55 μm wide, and 1.5-2.5 μm thick. Their spring constant was 0.1-0.2 N/m and their resonant frequency in an aqueous solution was 10-16 kHz. All AFM observations were performed at room temperature (24-26° C.). The images were acquired with 512 points by 512 lines. The scanning range was 110 μm×1100 μm and the scan time per line was 2 seconds. For force measurement, the viscoelastic properties of the cells were determined using force spectroscopy mode. In this mode, the AFM probe was moved in the vertical direction towards the cell surface, indented into the leaf surface and retracted away from the leaf surface right after the tip reached the targeted indentation force. The force curves were performed at three different areas of the same leaf for three different leaves with 8×8 force curves per area using the indentation force of 20 nN and the approaching velocity of approximately 2 μm/s. The scanning range was 27.5 μm×27.5 μm. All images and force distance curves were processed with the Nanosurf easyScan 2 3.8.0 software and the Young's modulus was extracted using AtomicJ 1.7.2 software²⁷.

### 3D Surface Mapping

Topographical images were directly taken from fresh, decellularized and recellularized leaves using a tactile sensor pad imaged with a GelSight, Inc. Benchtop System. The deformable gel elastomer pad (Medium-Firm, 20180524-001) was pressed onto each leaf. Six photographs were acquired by a standard DSLR camera (Canon Rebel T3i) with 18 megapixels resolved with a 5× lens (Canon MP-E 65 mm 1-5× Macro Lens). Each image is taken from a different angle illuminated with LED lighting. The images represent a 4.5 mm×3.0 mm area and are combined with GelSight software (GSCapture) to generate a textural map of the leaf surface.

### MTT Cell Viability Assay

A modified MTT experiment was developed from CellTiter 96 NonRadioactive Cell Proliferation Assay kit (Promega). Treated leaves were cut into 1×1 cm sized pieces and fit to the bottom of an untreated 48-well plate. 0.75×0.75 cm inserts were placed in the well on the top of the leaf, cells were plated at 10,000 cells/insert and incubated at 37° C. with 5% CO2 atmosphere. After 24 hours, the inserts were removed and leaves with attached cells were re-plated into non-treated 96-well plates. The medium was refreshed every 72 hours. Tetrazolium component was added at day 1, 3, 5 and 6 and absorbance of formazan product was measured at 570 nm by using Epoch microplate spectrophotometer (Biotek Instruments).

### Irradiation

All irradiation was performed using a cabinet X-ray machine (X-RAD 320, Precision X-Ray Inc., North Branford, Conn.) at 320 kVp and 12.5 mA with a 2 mm Al filter. Dose-rate was 3 Gy/min. The source-to-axis distance was 42 cm. The beam was calibrated using a UNIDOS E PTW T10010 electrometer and TN30013 ionization chamber, with measurement done in air, for a 15 cm×15 cm field size.

### Immunofluorescence Staining and Microscopy

Functionalized leaves were cut into 2×2 cm sized pieces and plated on an untreated 12-well plate with 1.75×1.75 cm inserts. Cells were seeded at 100,000 cells/insert and incubate for 24 hours. Cells were then fixed with 4% paraformaldehyde (PFA) in PBS for 15 min and permeabilized with 0.1% Triton X-100/PBS for 5 min. For γ-H2AX and 53BP1 foci formation assay, cell were fixed and permeabilized 30 minutes, 6 and 24 hours after 2 Gy-irradiation. Then, cells were blocked for 30 minutes at room temperature in 1% bovin serum albumin (BSA)/PBS and immunostained with antibodies diluted in 1% BSA/PBS to γH2AX Ser139 (JBW301; Upstate Cell Signaling; 1/800), 53BP1 (ab21083, Abcam, 1/500), vimentin (ab92547, Abcam, 1/100), pan-cytokeratin (ab7753, Abcam, 1/100), followed by Cy3-conjugated anti-mouse IgG (115-165-062, Jackson ImmunoResearch, 1/1000) and Alexa Fluor 647-conjugated anti-rabbit IgG (111-605-045, Jackson ImmunoResearch, 1/500) or Alexa Fluo 647-conjugated Phalloidin (#A22287, Life Technologies, 1/40) and counterstained with DAPI. Images were obtained using a Zeiss Axio Imager M2 epifluorescent microscope and were acquired with a Zeiss AxioCam MRm camera using ZEN 4.5 software at the Biomedical Imaging Core Facility at the UA College of Medicine—Phoenix.

### Gene Expression Assay

Treated leaves (2×2 cm pieces) were seeded with 500,000 cells/mL in an untreated 12-well plates and incubated for 72 hours. Then, cells were exposed to irradiation at either 1 Gy or 2 Gy. Six hours after irradiation, PC3 cells on the leaf were lysed and RNA was extracted using RNAspin Mini kit (GE Healthcare, 25-0500-71) according to the manufacturer's recommendation. RNA quantification was performed using Epoch microplate spectrophotometer (BioTek Instruments). RNA was stored at −80° C. until further use. RT-qPCR was then performed using RT2 Profiler PCR Array Human DNA Damage Signaling Pathway (Qiagen, #330231). Three hundred nanograms of total RNA were first used to generate cDNA using RT2 First Strand Kit according to manufacturer's instructions. Briefly, the reaction was first incubated at 42° C. for 5 minutes to eliminate residual genomic DNA and then placed one minute on ice. After addition of the reverse transcription mix, reaction was then placed at 42° C. for 15 additional minutes and stopped by incubation at 95° C. for 5 minutes before proceeding to PCR. RT2 Profiler Array PCR reactions were carried out on a 96-well plate format using a Stratagene Mx30005P (Agilent Technologies, Inc). The cDNA was added to the plate along with the RT Mastermix containing 2×RT2 SYBR Green. Cycling parameters were 10 min at 95° C. for initial denaturation, followed by 40 cycles of denaturation at 95° C. for 15 seconds and annealing and extension at 60° C. for 1 minute. Melting curves were automatically generated, ranging step-wise from 60 to 95° C. Data were collected by MxPro qPCR Software (Agilent). Values were normalized with actin, beta (ACTB); beta-2-microglobulin (B2M); glyceraldehyde-3-phosphate dehydrogenase (GAPDH); hypoxanthine phosphoribosyltransferase 1 (HPRT1) and ribosomal protein, large, P0 (RPLP0) and analyzed according to delta Ct method.²⁸ Genes were considered to be significantly expressed if the associated p-values were less than 0.05 and log 2-based expression change was greater than 1.5 or less than 0.66. The p-values for gene expression changes were calculated using Student's t-tests method.

### Colony Formation Assay

Cell survival was evaluated by colony formation assay. Cells were cultured both on leaf or in culture flasks for 3 days. Then, cells were harvested by trypsin, counted, seeded in triplicate into 6-well plates at appropriate cell densities and immediately irradiated. Then, cells were cultured for 14 days, and they were fixed with 6.0% glutaraldehyde and stained with 0.5% crystal violet. Colonies with more than 50 cells were counted and surviving fractions were calculated on the basis of the plating efficiencies of corresponding non-irradiated cells.

### Statistical Analysis

All statistical tests and graphs were performed with GraphPad Prism version 7.00 for Windows (GraphPad Software Inc., La Jolla, Calif.). ImageJ software was used to quantify γH2AX and 53BP1 foci. All results are presented as mean±SEM. Statistical comparisons were made by using Student's t-test. All differences were considered statistically significant when p<0.05.

### REFERENCES

1. Hanahan, D. & Weinberg, R. A. Hallmarks of cancer: the next generation. Cell 144, 646-674 (2011).
2. Fang, Y. & Eglen, R. M. Three-Dimensional Cell Cultures in Drug Discovery and Development. Slas Discov. 22, 456-472 (2017).
3. Eke, I. & Cordes, N. Radiobiology goes 3D: how ECM and cell morphology impact on cell survival after irradiation. Radiother. Oncol. J. Eur. Soc. Ther. Radiol. Oncol. 99, 271-278 (2011).
4. Acheva, A. et al. Human 3-D tissue models in radiation biology: current status and future perspectives. Int. J. Radiat. Res. 12, 81-98 (2014).
5. Gershlak, J. R. et al. Crossing kingdoms: Using decellularized plants as perfusable tissue engineering scaffolds. Biomaterials 125, 13-22 (2017).
6. Fontana, G. et al. Biofunctionalized Plants as Diverse Biomaterials for

Human Cell Culture. Adv. Healthc. Mater. 6, (2017).
7. Modulevsky, D. J., Lefebvre, C., Haase, K., Al-Rekabi, Z. & Pelling, A. E. Apple Derived Cellulose Scaffolds for 3D Mammalian Cell Culture. PLOS ONE 9, e97835 (2014).
8. Vogel, V. Unraveling the Mechanobiology of Extracellular Matrix. Annu. Rev. Physiol. 80, 353-387 (2018).
9. Theocharis, A. D., Skandalis, S. S., Gialeli, C. & Karamanos, N. K.

Extracellular matrix structure. Adv. Drug Deliv. Rev. 97, 4-27 (2016).
10. Kular, J. K., Basu, S. & Sharma, R. I. The extracellular matrix: Structure, composition, age-related differences, tools for analysis and applications for tissue engineering. J. Tissue Eng. 5, 2041731414557112 (2014).
11. Johnson, M. K., Cole, F., Raj, A. & Adelson, E. H. Microgeometry capture using an elastomeric sensor. ACM Trans Graph 30, 46-46 (2011).
12. Lacombe, J., Sima, C., Amundson, S. A. & Zenhausern, F. Candidate gene biodosimetry markers of exposure to external ionizing radiation in human blood: A systematic review. PloS One 13, e0198851 (2018).
13. Lu, T.-P., Hsu, Y.-Y., Lai, L.-C., Tsai, M.-H. & Chuang, E. Y. Identification of Gene Expression Biomarkers for Predicting Radiation Exposure. Sci. Rep. 4, 6293 (2014).
14. Macaeva, E. et al. Radiation-induced alternative transcription and splicing events and their applicability to practical biodosimetry. Sci. Rep. 6, 19251 (2016).
15. Paul, S. & Amundson, S. A. Development of gene expression signatures for practical radiation biodosimetry. Int. J. Radiat. Oncol. Biol. Phys. 71, 1236-1244 (2008).
16. Jain, R. K. et al. Biomarkers of response and resistance to antiangiogenic therapy. Nat. Rev. Clin. Oncol. 6, 327-338 (2009).
17. Chen, F.-H. et al. Radiotherapy Decreases Vascular Density and Causes Hypoxia with Macrophage Aggregation in TRAMP-C1 Prostate Tumors. Clin. Cancer Res. Off. J. Am. Assoc. Cancer Res. 15, 1721-1729 (2009).
18. Baish, J. W. et al. Scaling rules for diffusive drug delivery in tumor and normal tissues. Proc. Natl. Acad. Sci. U.S.A. 108, 1799-1803 (2011).
19. Katira, P., Bonnecaze, R. T. & Zaman, M. H. Modeling the Mechanics of Cancer: Effect of Changes in Cellular and Extra-Cellular Mechanical Properties. Front. Oncol. 3, (2013).
20. Emon, B., Bauer, J., Jain, Y., Jung, B. & Saif, T. Biophysics of Tumor Microenvironment and Cancer Metastasis—A Mini Review. Comput. Struct. Biotechnol. J. 16, 279-287 (2018).
21. Wang, S., Ren, L., Liu, Y., Han, Z. & Yang, Y. Mechanical Characteristics of Typical Plant Leaves. J. Bionic Eng. 7, 294-300 (2010).
22. Lacombe, J., Phillips, S. L. & Zenhausern, F. Microfluidics as a new tool in radiation biology. Cancer Lett. 371, 292-300 (2016).
23. Najafi, M., Fardid, R., Hadadi, G. & Fardid, M. The Mechanisms of Radiation-Induced Bystander Effect. J. Biomed. Phys. Eng. 4, 163-172 (2014).
24. EL-Ashhab, F., Sheha, L., Abdalkhalek, M. & Khalaf, H. A. The influence of gamma irradiation on the intrinsic properties of cellulose acetate polymers. J. Assoc. Arab Univ. Basic Appl. Sci. 14, 46-50 (2013).
25. Henniges, U., Okubayashi, S., Rosenau, T. & Potthast, A. Irradiation of Cellulosic Pulps: Understanding Its Impact on Cellulose Oxidation. Biomacromolecules 13, 4171-4178 (2012).
26. Polvi, J. et al. Primary Radiation Defect Production in Polyethylene and Cellulose. J. Phys. Chem. B 116, 13932-13938 (2012).
27. Hermanowicz, P., Sarna, M., Burda, K. & Gabryś, H. AtomicJ: an open source software for analysis of force curves. Rev. Sci. Instrum. 85, 063703 (2014).
28. Livak, K. J. & Schmittgen, T. D. Analysis of relative gene expression data using real-time quantitative PCR and the 2(−Delta Delta C(T)) Method. Methods San Diego Calif. 25, 402-408 (2001).
WO 2017/160862 to Murphy et al., 21 Sep. 2017

### Example 3: Supercritical Fluid Decellularization Methods for Plant-Derived Cellulose Scaffolds

It has been discovered that decellularization of plant material via supercritical fluid may be faster and able to better preserve tissue structure and mechanical properties than conventional methods that use detergents, such as sodium dodecyl sulfate (SDS) and Triton X-100. In particular, it has been discovered that supercritical CO₂ (sfCO₂) with peracetic acid (PAA) and ethanol may be particularly useful in decellularizing plant material quickly and efficiently while also preserving the cellulosic structure.

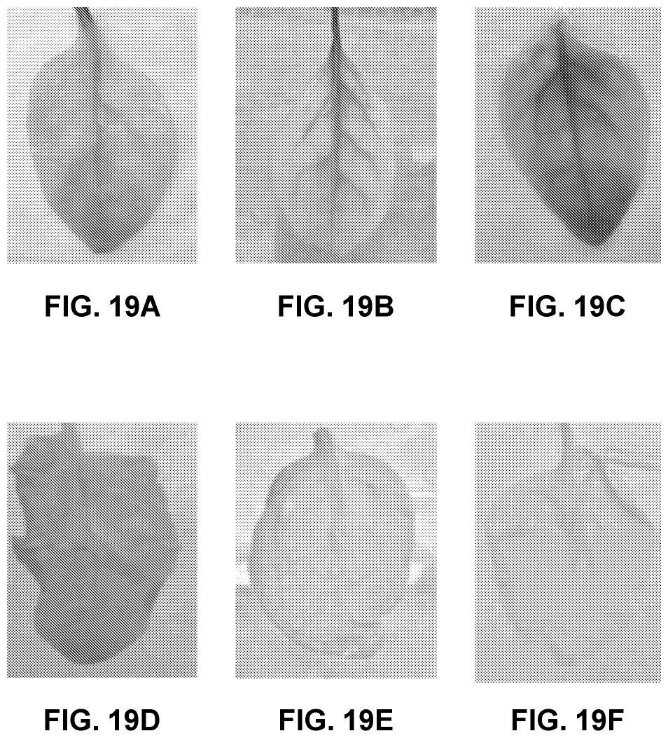

Different sfCO₂ co-solvents were evaluated FIGS. 19A-19F show the results of leaf decellularization via various sfCO₂ co-solvents. Spinach leaves were visually inspected after being treated with supercritical CO₂ with various weak base co-solvents. Ethanol and combinations of peracetic acid (PAA) were the most effective at removing plant material (FIGS. 19D-19F) while bleach was the least effective (FIG. 19C) at removing plant contents such as chlorophyll. The sfCO₂ decellularization method was initiated by soaking the plant material in a hexane bath for 5 minutes to remove the wax cuticle, followed by two washes with PBS. Each individual leaf was placed in its own vented 15-ml conical tube and 5 mL of co-solvent was added directly to each tube. The lids were fastened onto the tubes, yet left loose to allow more opportunity for the fluid to penetrate the tube. A maximum of three tubes were placed into the 600 m L pressure vessel of a NovaGenesis500 (Novasterilis, N.Y.) and sealed with 25-foot pounds of torque. The leaves were exposed to scCO₂ at 2500 psi, 33° C. for three hours. Time clock was initiated when the pressure and temperature equalized. FIG. 19A shows a spinach leaf after being treated with supercritical CO₂ and 75% EtOH. FIG. 19B shows a spinach leaf after being treated with supercritical CO₂ and 5% SDS in 75% EtOH. FIG. 19C shows a spinach leaf after being treated with supercritical CO₂ and 10% Bleach in 75% EtOH. FIG. 19D shows a spinach leaf after being treated with supercritical CO₂ and 1% PAA in 75% EtOH. FIG. 19E shows a spinach leaf after being treated with supercritical CO₂ and 2% PAA in 75% EtOH. FIG. 19F shows a spinach leaf after being treated with supercritical CO₂ and 3% PAA in 75% EtOH.

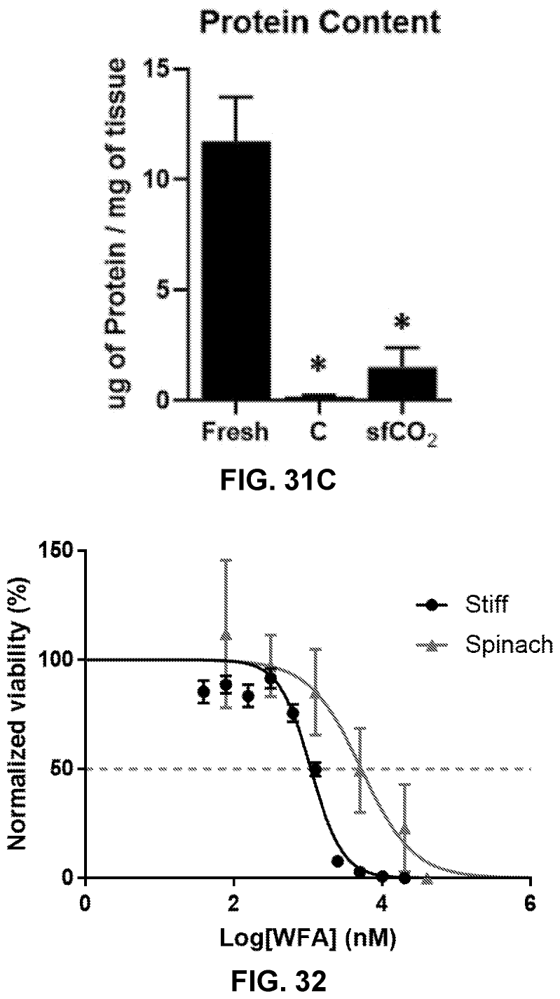

Treatment time may range between 1 and 3 hours at 1500-2500 psi and 33-34° C. Particularly useful methods of decellularization include using 5 ml of co-solvent containing 75% EtOH and 3% PAA at 2500 psi, 33° C. for three hours. This method efficiently decellularizes vegetal materials, as shown in FIGS. 31A-31C.

### Example 4: Mimicking of a Biological Tissue

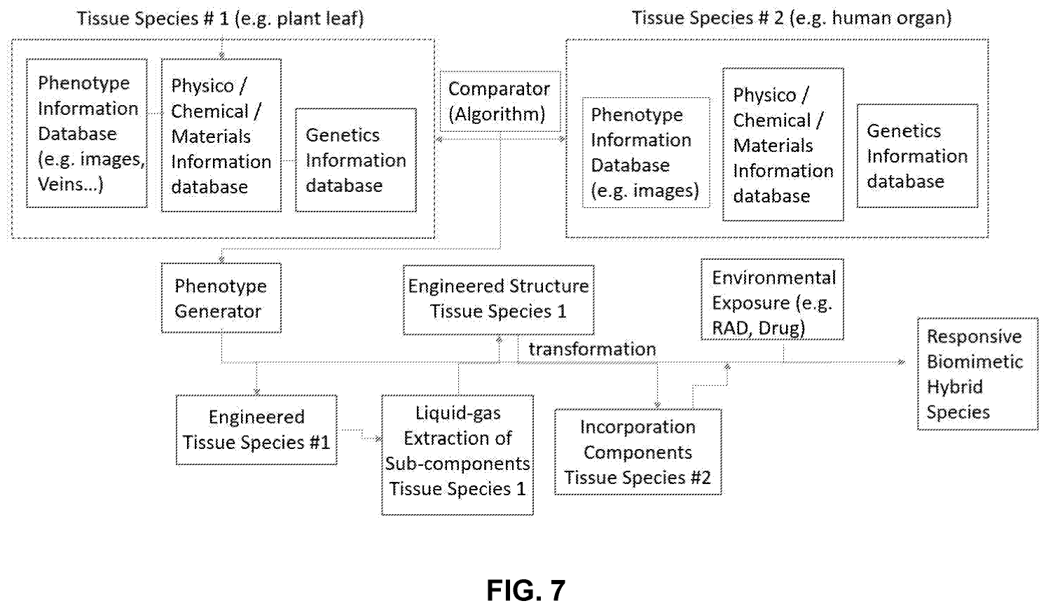

Referring to FIG. 7, an exemplary method of mimicking a biological tissue is schematically illustrated by a flow-chart. In the method, a First Tissue Species database includes a Phenotype Information Database, a Physico/Chemical/Materials information database and a Genetic Information database. Genetic Information database may include genetic information related to the plurality of different plant species.

In some embodiments, the information stored in the Phenotype Information Database of the First Tissue Species database may include information corresponding to the vascular morphology of each of the plurality of plant species. In one example, the vascular morphology information may include vascular density (e.g., number of vessels per unit area for a 2D representation or number of vessels per unit volume for a 3D representation), vascular branching (e.g., a mathematical formula that approximates the branching morphology, such as shape, angle, density, spacing, etc.), vascular capacity (e.g., friction losses of fluid flowing inside the leaf vasculature), among others, that characterizes the vasculature of a plant tissue of the database. Representative plants include any of a range of plants that may have a geometry and scaffold useful for mimicking mammalian tissue.

In some embodiments, the information stored in the Physico/Chemical/Materials information database of the First Tissue Species database may include information corresponding to biomechanical properties of each of the plurality of plant species. In one example, the biomechanical properties may include elastic modulus (i.e., Young's modulus), ultimate tensile strength, among others, that characterizes the biomechanical properties of a plant tissue of the database.

In some embodiments, the information stored in the Genetic Information database of the First Tissue Species database may include genetic information related to the plurality of different plant species. In one example, the information stored in the Genetic Information database may include DNA or RNA, for example DNA sequence information that characterizes the genetic information of a plant tissue of the database.

The Second Tissue Species database may include a Phenotype Information Database, a Physico/Chemical/Materials information database and a Genetic Information database related to a plurality of different mammalian tissue types (e.g., different human organs). In one embodiment, the First Tissue Species database stores information related to tissue of a plurality of different human organs. The information stored in the Phenotype Information Database may include morphology properties related to the plurality of different human organs. The information in the Physico/Chemical/Materials information database may include biomechanical and/or chemical properties related to the plurality of different human organs. The Genetic Information database may include genetic information related to the plurality of different human organs.

In some embodiments, the information stored in the Phenotype Information Database of the Second Tissue Species database may include information corresponding to the vascular morphology of each of the plurality of human organs. In one example, the vascular morphology information may include vascular density (e.g., number of vessels per unit area for a 2D representation of tissue of a human heart or number of vessels per unit volume for a 3D representation of tissue of a human heart), vascular branching (e.g., a mathematical formula that approximates the branching morphology of tissue of a human heart), vascular capacity (e.g., friction losses of fluid flowing inside the tissue of a human heart) among others, that characterizes the vasculature of a human tissue of the database.

In some embodiments, the information stored in the Physico/Chemical/Materials information database of the Second Tissue Species database may include information corresponding to biomechanical properties of each of the human tissue types. In one example, the biomechanical properties may include elastic modulus (i.e., Young's modulus), ultimate tensile strength, among others, that characterizes the biomechanical properties of a human tissue of the database.

In some embodiments, the information stored in the Genetic Information database of the Second Tissue Species database may include genetic information related to the plurality of different human tissues. In one example, the information stored in the Genetic Information database may include DNA or RNA, for example DNA sequence information among others, that characterizes the genetic information of a human tissue of the database.

In the method, a human tissue type may be selected. As shown in FIG. 7, a Comparator may compare the information stored in the First database to the information stored in the Second database to statistically identify a plant tissue of best fit for the selected human tissue. Next, the selected plant tissue is provided and undergoes a liquid-gas extraction of sub-components to produce an engineered structure, such as a decellularized vascularized cellulose scaffold. Next, components of the selected human tissue are incorporated into the engineered structure, for example human cells may be seeded onto the vascularized cellulose scaffold to produce an analogue vascular mammalian (human) tissue. Next, the analogue vascular mammalian tissue may be exposed to environmental stimulus, such as radiation or one or more drugs.

### Example 5: Development of Biomechanically Tunable Plant-Based Scaffolds to Study Cell-Substrate Interactions

In vitro cell culture models of the prior art do not accurately recapitulate the mechanical characteristics, in particular stiffness, of in vivo tissue. Thus, plant-based scaffolds with different stiffness may be used to reproduce the broad range of mechanical properties of human tissue. Several plant species were decellularized and the resulting scaffolds displayed different stiffness starting from few kPa, much softer than traditional tissue culture polystyrene (TCPS). Melanoma SK-MEL-28 and prostate PC3 cells were then seeded on spinach scaffolds to compare mechanoregulation with cells seeded on TCPS. Results showed that YAP/TAZ signaling was inactivated, cellular morphology altered and proliferation rate decreased when cells were cultured on leaf scaffold. Unexpectedly, cell culture on soft vegetal scaffold also affected cellular response to external stress. Thus, SK-MEL-28 cells phenotype is modified leading to a drug resistance while PC3 cells showed altered gene expression and radiation response.

Thus, decellularized plant-based scaffolds may reproduce the broad range of human tissue stiffness, and thus be used to as an alternative to standard cell culture to better mimic mechanical properties of different disease or healthy tissue environment. The activation of mechanotransduction pathways of cells seeded on the leaf was compared to cells seeded in standard plastic cell culture flasks. In one aspect, these scaffolds may be used as new cellular models for drug screening and treatment response. In one aspect the selection of the particular plant-based scaffold may impact cellular response to external stimuli such as drug response in melanoma cells or radiation response in prostate cancer cells compared to standard cell culture models.

Plant material, including baby spinach leaves (Spinacia oleracea), cherry tomato plant leaf (Solanum lycopersicum), aquatic plant (Echinodorus grisebachii) and A. borealis (Kalanchoe fedtschenkoi variegati) were acquired. To initiate decellularization, spinach, tomato and aquatic leaves were cannulated through the petiole (base of the stem) with a 26-gauge needle and secured with heat shrink tubing. A. borealis was not cannulated but directly soaked in the different solutions on an orbital shaker. The wax cuticle protecting the leaf was removed with 3 cycles of alternating washes with hexanes and phosphate-buffered saline (PBS). The prepared leaves were connected to gravity bags filled with the different solutions. Decellularization by chemical treatment started with perfusion of a solution of 1% sodium dodecyl sulfate (SDS) in deionized (DI) water for 2 days, followed by a solution of 10% sodium chlorite and 0.1% Triton-X 100 in DI water for 2 days. The leaves were then flushed with DI water for an additional 48 hours. Leaves were stored in DI water at 4 degrees Celsius (° C.) until ready for use.

DNA/Protein Quantification

Leaves were first placed in centrifuge tubes and then were disrupted after freezing in liquid nitrogen by using a pestle. Resulting powder was transferred to a microcentrifuge tube for DNA and protein quantification. DNA content of fresh and decellularized leaves was extracted using DNeasy Plant Mini Kit (Qiagen) following manufacturer recommendation, while proteins were extracted by radioimmunoprecipitation assay (RIPA) (Pierce RIPA buffer, ThermoFisher Scientific) for 30 min on ice. DNA content was quantified by reading absorbance at 260 nm and protein content was quantified using microBCA protein assay kit (ThermoFisher Scientific). Both absorbances were measured using Epoch microplate spectrophotometer (BioTek Instruments).

Human Cell Culture and Seeding on Leaf Scaffold

Prostate cancer cells (PC3) and melanoma cells (SK-MEL-28) were obtained from ATCC (CRL-1435 and HTB-72, respectively). PC3 cells were cultured in Roswell Park Memorial Institute 1640 medium and SK-MEL-28 in Minimum Essential Medium, both supplemented with 10% fetal bovine serum (FBS) and 1% Penicillin/Streptomycin (P/S). Prior to recellularization, spinach leaf scaffolds were first sterilized using a UV Stratalinker 2400 (Stratagene) for 30 minutes. Leaf structures were then functionalized with collagen and fibronectin proteins. Briefly, scaffolds were incubated in 50 ug/ml of collagen I (A1048301, Thermo Scientific) in 20 mM acetic acid solution for 4 hours, followed by two washes in PBS and a final wash in complete medium. Leaves were then incubated in 10 ug/ml fibronectin (F0895, Sigma-Aldrich) for 24 hours followed by three washes in complete medium. Finally, treated leaves were cut into small pieces and fit to the bottom of untreated multiple well plates. Inserts were placed in the well, on the top of the leaf, to contain the cells and facilitate cellular attachment. Cell attachment was promoted for different periods of time, depending on the assay, at 37° C. with 5% CO₂ atmosphere.

Atomic Force Microscopy (AFM)-Based Imaging and Force Mapping

AFM instrument was Nanosurf LensAFM (Nanosurf, Switzerland) coupled with an upright Nikon Eclipse E800 optical microscope (Nikon, Japan). The SCM-PIC cantilevers (Bruker) were 405-495 μm long, 45-55 μm wide, and 1.5-2.5 μm thick. Their spring constant was 0.1-0.2 N/m and their resonant frequency in an aqueous solution was 10-16 kHz. All AFM observations were performed at room temperature (24-26° C.). The images were acquired with 512 points by 512 lines. The scanning range was 87.5 μm×87.5 μm and the scan time per line was 2 seconds. For force measurement, the viscoelastic properties of the cells were determined using force spectroscopy mode. In this mode, the AFM probe was moved in the vertical direction towards the cell surface, indented into the leaf surface and retracted away from the leaf surface right after the tip reached the targeted indentation force. The force curves were performed at three different areas of the same leaf for three different leaves with 8×8 force curves per area using the indentation force of 20 nN and the approaching velocity of approximately 2 μm/s. The scanning range was 27.5 μm×27.5 μm. The force-distance curves of stiff substrate were acquired by using a PPP-FMR9-SPL1 cantilevers (Bruker) that are 225 μm long, 28 μm wide with a spring constant of 2 N/m and a frequency of 75 kHz. All images and force distance curves were processed with the Nanosurf easyScan 2 3.8.0 software and the Young's modulus was extracted using AtomicJ 1.7.2 software, assuming a Poisson's ratio of 0.5 [12].

3D Surface Mapping

Topographical images were directly taken from fresh, decellularized and recellularized leaves using a tactile sensor pad imaged with a GelSight, Inc. Benchtop System. The deformable gel elastomer pad (Medium-Firm, 20180524-001) was pressed onto each leaf. Six photographs were acquired by a standard DSLR camera (Canon Rebel T3i) with 18 megapixels resolved with a 5× lens (Canon MP-E 65 mm 1-5× Macro Lens). Each image is taken from a different angle illuminated with LED lighting. The images represent a 4.5 mm×3.0 mm area and are combined with GelSight software (GSCapture) to generate a textural map of the leaf surface.

MTT Cell Viability Assay

A modified MTT experiment was developed from CellTiter 96 NonRadioactive Cell Proliferation Assay kit (Promega). Treated leaves were cut into 1×1 cm sized pieces and fit to the bottom of an untreated 48-well plate. 0.75×0.75 cm inserts were placed in the well on the top of the leaf, cells were plated at 10,000 cells/insert and incubated at 37° C. with 5% CO2 atmosphere. After 24 hours, the inserts were removed and leaves with attached cells were cut and re-plated into non-treated 96-well plates. In parallel, cells were also directly seeded into a treated 96-well plate at a concentration of 5000 cells/well. The medium was refreshed every 72 hours. Tetrazolium component was added at day 1, 3, 5 and 7 and absorbance of formazan product was measured at 570 nm by using Epoch microplate spectrophotometer (Biotek Instruments). For the drug response assay, withaferin A (WFA) was added at concentrations ranging from 0.156 to 40 μM and incubated for 72 hours prior to the addition of the formazan product. DMSO (0.8%) served as vehicle and control. The concentration of drugs that resulted in 50% of cell death (IC₅₀) was determined from dose-response curve by using PRISM 7.0 (GraphPad100 Software, San Diego, Calif., USA). Experiments were repeated three times, and data represented as the mean of quadruplicate wells±SEM.

Immunoblotting

For protein extraction, recellularized leaf scaffolds and cells in TCPS at 80% confluency were first washed three times with PBS to remove excess of cell culture medium. The leaf scaffold was then immerged in RIPA buffer for 30 min on ice with regular vortex steps while RIPA was directly added into the TCPS for 15 min on ice before cell scrapping. Cell lysates were then centrifuged at maximal speed, supernatants were collected and protein contents were quantified using microBCA protein assay kit (ThermoFisher Scientific). Absorbance was read using an Epoch microplate spectrophotometer (BioTek Instruments). Samples (5 μg total protein) were mixed with Laemmli sample buffer (Bio-Rad) and heated at 95° C. for 5 minutes. Samples were then loaded on graded pre-cast polyacrylamide gels (4-20% mini PROTEAN TGX gels, Bio-Rad), separated based on size by electrophoresis (90V for 2 hours) and transferred (300 mA for 90 min) to PVDF membranes (Bio-Rad). Membranes were then blocked with PBS plus 0.1% Tween-20 containing 5% dried non-fat milk at room temperature for one hour. Blots were then incubated at 4° C. overnight with primary antibodies against Yorkie-homologues/yes-associated protein−1 (YAP) (sc101199; Santa Cruz; 1/200) and transcriptional coactivator with PDZ-binding motif (TAZ) (#4883; Cell Signaling; 1/1000). Glyceraldehyde 3-phosphate dehydrogenase (GAPDH) (sc365062; Santa Cruz; 1/200). was used as the control. After five washes (5 min/each) in PBS plus Tween-20, membranes were incubated with anti-mouse HRP-conjugated antibody (1/10,000; Jackson ImmunoResearch Laboratories) or anti-rabbit HRP-conjugated antibody (1/5,000; Jackson ImmunoResearch Laboratories) at room temperature for one hour. After five additional washes, membranes were developed with ECL substrate (Clarity Western, Bio-Rad) and imaged using a GBox Chemi doc system (XX6, Syngene). Band intensities were quantified by using ImageJ and differences between experimental conditions were analyzed using the Mann-Whitney test.

Real-Time Quantitative Reverse Transcription PCR (qRT-PCR)

Treated leaves (2×2 cm pieces) were seeded with 500,000 cells/mL in an untreated 12-well plate and incubated for 72 hours. In parallel, cells were also grown in TCPS until 80% confluency. Cells on the leaf or in TCPS were lysed and RNA was extracted using RNAspin Mini kit (GE Healthcare, 25-0500-71) according to the manufacturer's recommendation. RNA quantification was performed using Epoch microplate spectrophotometer (BioTek Instruments). RNA was stored at −80° C. until further use. One microgram of total RNA was first used to generate cDNA using a QuantiTect Reverse Transcriptase kit (Qiagen, #205310) according to manufacturer's instructions. Briefly, the reaction was first incubated at 42° C. for 2 minutes to eliminate residual genomic DNA and then placed immediately on ice. After addition of the reverse transcription mix, reaction was then placed at 42° C. for 15 additional minutes and stopped by incubation at 95° C. for 3 minutes before proceeding to PCR. QuantiFast SYBR Green PCR reactions (Qiagen, #204054) were carried out on a 96-well plate format using a Stratagene Mx30005P (Agilent Technologies, Inc). The cDNA was added to the plate along with the RT Mastermix containing 2× QuantiFast SYBR Green PCR Master Mix, RNase-free water and primers (1 μM): ANKRD1 (F: AGAACTGTGCTGGGAAGACG (SEQ ID NO:1); R: GCCATGCCTTCAAAATGCCA (SEQ ID NO:2)), CTGF (F: AGGAGTGGGTGTGTGACGA (SEQ ID NO:3); R: CCAGGCAGTTGGCTCTAATC (SEQ ID NO:4)), GAPDH (F: CTCCTGCACCACCAACTGCT (SEQ ID NO:5); R: GGGCCATCCACAGTCTTCTG (SEQ ID NO:6)) MITF (F: GTTGCCTGTCTCGGGAAACT (SEQ ID NO:7); R: TACACGCTGTGAGCTCCCTT (SEQ ID NO:8)), MLANA (F: GGGAGTCTTACTGCTCATCGG (SEQ ID NO:9); R: TCAAACCCTTCTTGTGGGCA (SEQ ID NO:10)), SOX10 (F: GGAGGCTGCTGAACGAAAGT (SEQ ID NO:11); R: GGGCGCTCTTGTAGTGGG (SEQ ID NO:12)), TAZ (F: GGACCAAGTACATGAACCACC (SEQ ID NO:13); R: TGCAGGACTGGTGATTGGAC (SEQ ID NO:14)), TYR (F: CGAGTCGGATCTGGTCATGG (SEQ ID NO:15); R: GACACAGCAAGCTCACAAGC (SEQ ID NO:16)) and YAP (F: CCCTCGTTTTGCCATGAACC (SEQ ID NO:17); R: GTTGCTGCTGGTTGGAGTTG (SEQ ID NO:18)). Cycling parameters were 5 min at 95° C. for initial activation, followed by 40 cycles of denaturation at 95° C. for 10 seconds and combined annealing and extension at 60° C. for 30 seconds. Melting curves were automatically generated, ranging step-wise from 60 to 95° C. Data were collected by MxPro qPCR Software (Agilent). Values were normalized with glyceraldehyde-3-phosphate dehydrogenase (GAPDH) and analyzed according to delta Ct method [13].

Scanning Electron Microscopy (SEM)

Functionalized leaves were cut into 1×1 cm sized pieces and plated on an untreated 24-well plate with 0.75×0.75 cm inserts. Cells were seeded at 50,000 cells/insert and incubated for 72 hours. In parallel, cells were also seeded on coverslip at a concentration of 12 000 cells/cm2 for 72 hours. Cells were fixed with 2.5% glutaraldehyde in 1×PBS overnight at 4° C. Samples were dehydrated in increasing concentrations of ethanol (50, 70, 85, 95, 95, 100, 100) for one hour each and left overnight in 100% ethanol at 4° C. Samples were left to dry on pin stub mounts (12.7×8 mm, Ted Pella) and sputter coated with gold (10 nm) prior imaging to the Eyring Materials Center at Arizona State University with a SEM-FEG XL30 (FEI).

Irradiation

All irradiation was performed using a cabinet X-ray machine (X-RAD 320, Precision X-Ray Inc., North Branford, Conn.) at 320 kVp and 12.5 mA with a 2 mm Al filter. Dose-rate was 3 Gy/min. The source-to-axis distance was 42 cm. The beam was calibrated using a UNIDOS E PTW T10010 electrometer and TN30013 ionization chamber, with measurement done in air, for a 15 cm×15 cm field size.

Immunofluorescence Staining and Microscopy

Functionalized leaves were cut into 2×2 cm sized pieces and plated on an untreated 12-well plate with 1.75×1.75 cm inserts. Cells were seeded at 100,000 cells/insert and incubated for 72 hours. In parallel, cells were also seeded on coverslip at a concentration of 12 000 cells/cm² for 72 hours. Cells were then fixed with 4% paraformaldehyde (PFA) in PBS for 15 min and permeabilized with 0.1% Triton X-100/PBS for 5 min. For γ-H2AX foci formation assay, cells were fixed and permeabilized at 1, 6 and 24 hours after 2 Gy-irradiation. Then, cells were blocked for 30 minutes at room temperature in 1% bovin serum albumin (BSA)/PBS and immunostained with antibodies diluted in 1% BSA/PBS to γH2AX Ser139 (JBW301; Upstate Cell Signaling; 1/800), alpha-tubulin (sc5286; Santa Cruz; 1/150), beta-catenin (sc7963; Santa Cruz; 1/100), YAP (sc-101199; Santa Cruz; 1/50) and Ki-67 (ab15580; AbCam; 1/1000) followed by Cy3-conjugated anti-mouse IgG (115-165-062, Jackson ImmunoResearch, 1/1000) and Alexa Fluor 647-conjugated anti-rabbit IgG (111-605-045, Jackson ImmunoResearch, 1/500) or Alexa Fluo 647-conjugated Phalloidin (#A22287, Life Technologies, 1/40) and counterstained with DAPI. Images were obtained using a Zeiss Axio Imager M2 epifluorescent microscope and were acquired with a Zeiss AxioCam MRm camera using ZEN 4.5 software at the Biomedical Imaging Core Facility at the UA College of Medicine—Phoenix.

DNA Damage Signaling Pathway Assay

Treated leaves (2×2 cm pieces) were seeded with 500,000 cells/mL in an untreated 12-well plate and incubated for 72 hours. In parallel, cells were also grown in TCPS until 80% confluency. Then, cells were exposed to irradiation at 2 Gy. Six hours after irradiation, PC3 cells in TCPS were harvested and lyzed, while cells on the leaf were directly lyzed on the scaffold. RNA was extracted using RNAspin Mini kit (GE Healthcare, 25-0500-71) according to the manufacturer's recommendation. RNA quantification was performed using Epoch microplate spectrophotometer (BioTek Instruments). RNA was stored at −80° C. until further use. qRT-PCR was then performed using RT₂ Profiler PCR Array Human DNA Damage Signaling Pathway (Qiagen, #330231). Three hundred nanograms of total RNA were first used to generate cDNA using RT² First Strand Kit according to manufacturer's instructions. Briefly, the reaction was first incubated at 42° C. for 5 minutes to eliminate residual genomic DNA and then placed one minute on ice. After addition of the reverse transcription mix, reaction was then placed at 42° C. for 15 additional minutes and stopped by incubation at 95° C. for 5 minutes before proceeding to PCR. RT² Profiler Array PCR reactions were carried out on a 96-well plate format using a Stratagene Mx30005P (Agilent Technologies, Inc). The cDNA was added to the plate along with the RT Mastermix containing 2×RT2 SYBR Green. Cycling parameters were 10 min at 95° C. for initial denaturation, followed by 40 cycles of denaturation at 95° C. for 15 seconds and annealing and extension at 60° C. for 1 minute. Melting curves were automatically generated, ranging step-wise from 60 to 95° C. Data were collected by MxPro qPCR Software (Agilent). Values were normalized with actin, beta (ACTB); beta-2-microglobulin (B2M); glyceraldehyde-3-phosphate dehydrogenase (GAPDH); hypoxanthine phosphoribosyltransferase 1 (HPRT1) and ribosomal protein, large, P0 (RPLP0) and analyzed according to delta Ct method [13]. Gene expression were considered to be significantly modified if the associated p-values were less than 0.05 and fold changes (FC) was greater than 1.5 or less than 0.66.

Clonogenic Assay

Cells were cultured both on spinach leaf or in TCPS for 3 days. Then, cells were harvested by trypsin, counted, seeded in triplicate into 6-well plates at appropriate cell densities and immediately irradiated at 0, 2, 4 and 6 Gy. Then, cells were cultured for 14 days, and they were fixed with 6.0% glutaraldehyde and stained with 0.5% crystal violet. Colonies with more than 50 cells were counted and surviving fractions were calculated on the basis of the plating efficiencies of corresponding non-irradiated cells.

Statistical Analysis

All statistical tests and graphs were performed with GraphPad Prism version 7.00 for Windows (GraphPad Software Inc., La Jolla, Calif.). ImageJ software was used to quantify γH2AX and 53BP1 foci, immunoblot bands intensities and cell spreading areas. All results are presented as mean±SEM. Statistical comparisons were made by using Student's t-test or Mann-Whitney test if data distribution did not pass normality test. All differences were considered statistically significant when p<0.05.

Decellularized Vegetal Scaffolds Display a Broad Range of Stiffness

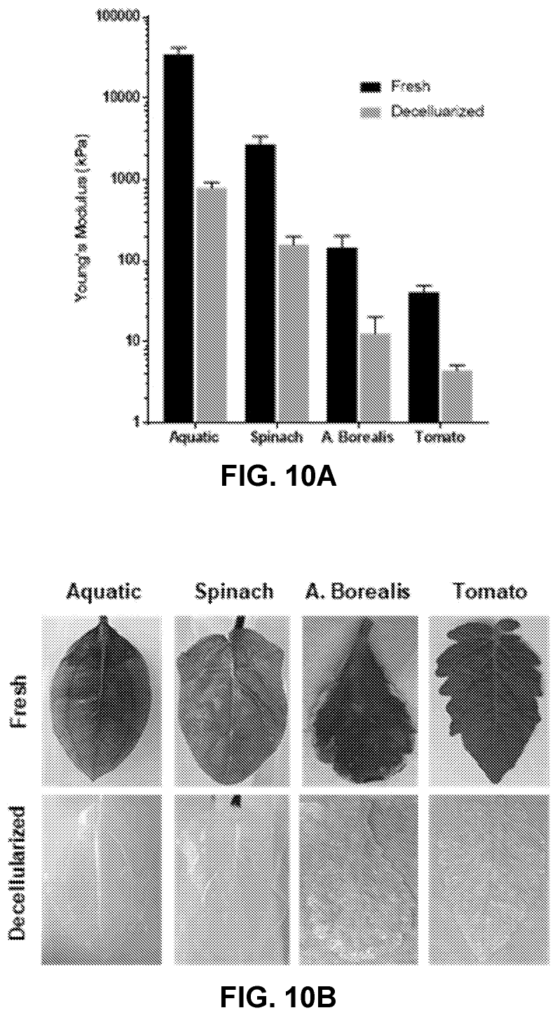

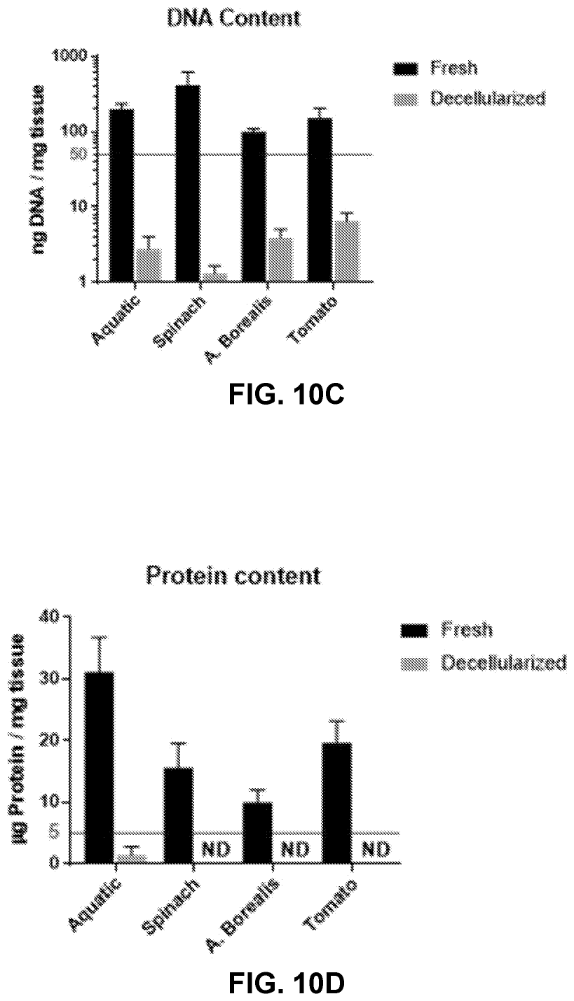

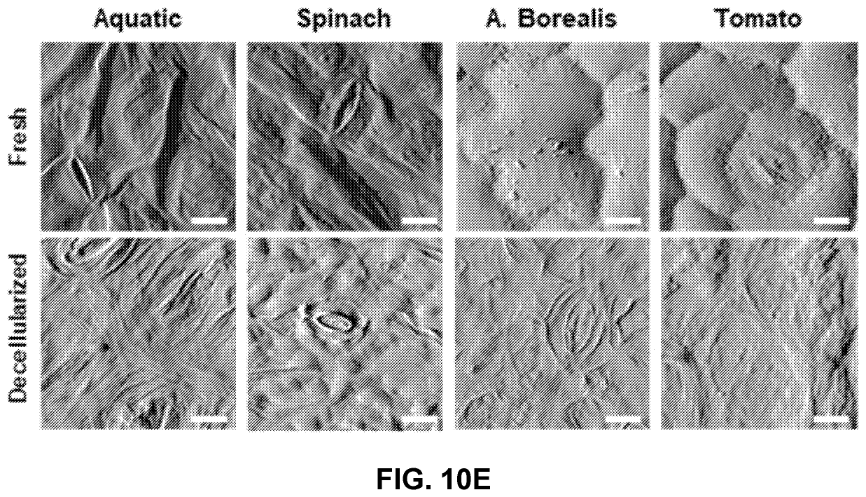

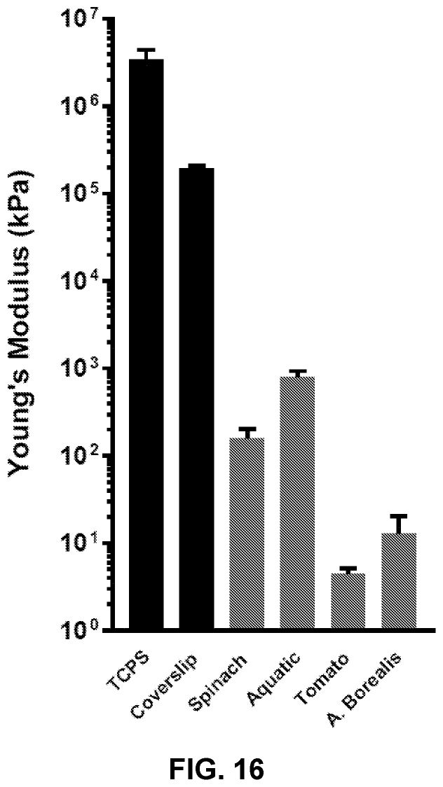

Vegetal structures with different Young Modulus (YM) as measured by atomic force microscopy (AFM) were decellularized by serial chemical treatment. Among the native plants, the aquatic plant Echinodorus grisebachii leaves presented the highest YM (34±7 MPa) and tomato (Solanum lycopersicum) leaves the lowest YM (41±21 kPa), while A. borealis (Kalanchoe fedtschenkoi variegata “Aurora borealis”) and spinach (Spinacia oleracea) leaves had intermediate YM (147±55 kPa and 17±8 MPa respectively) (FIG. 10A). After 7 days of treatment, all vegetal structures lost chlorophyll and appeared fully translucent (FIG. 10B) suggesting that plant material have been successfully removed from the native structure. To assess the efficiency of this decellularization process, protein and DNA content was then quantified. Decellularized leaves contained significantly less DNA (FIG. 10C) and protein (FIG. 10D) compared to fresh leaves, below the minimal requirement to consider a tissue as decellularized [14]. In addition, AFM-based imaging showed that plant structures, such as stoma and veins, are more visible on the decellularized leaf compared to fresh leaf (FIG. 10E). Altogether, these data suggest that decellularization treatment was effective to remove all vegetal content. AFM-based force mapping performed on resulting scaffolds showed that YM of decellularized structures were all lower than native structures confirming that plant structures were soften by chemical decellularization treatment (FIG. 10A). Interestingly, after decellularization, vegetal structures still displayed a broad range of stiffness, with YM of 794±131, 158±44, 13±7 and 4±0.6 kPa for aquatic, spinach, A. borealis and tomato leaves respectively. This suggests that resulting scaffolds may provide more appropriate matrixes to reproduce the mechanical properties of different human tissue than standard tissue culture polystyrene (TCPS) flasks or coverslips which displayed much higher YM (FIG. 16). To better characterize the biomechanical patterning of the cells seeded on vegetal scaffolds, mechanosensitive pathways of the cells seeded on the scaffolds were compared to those of cells seeded on stiff substrate.

YAP/TAZ Signaling Pathway is Inactivated in Cells Cultured on Spinach Leaf Decellularized Scaffolds

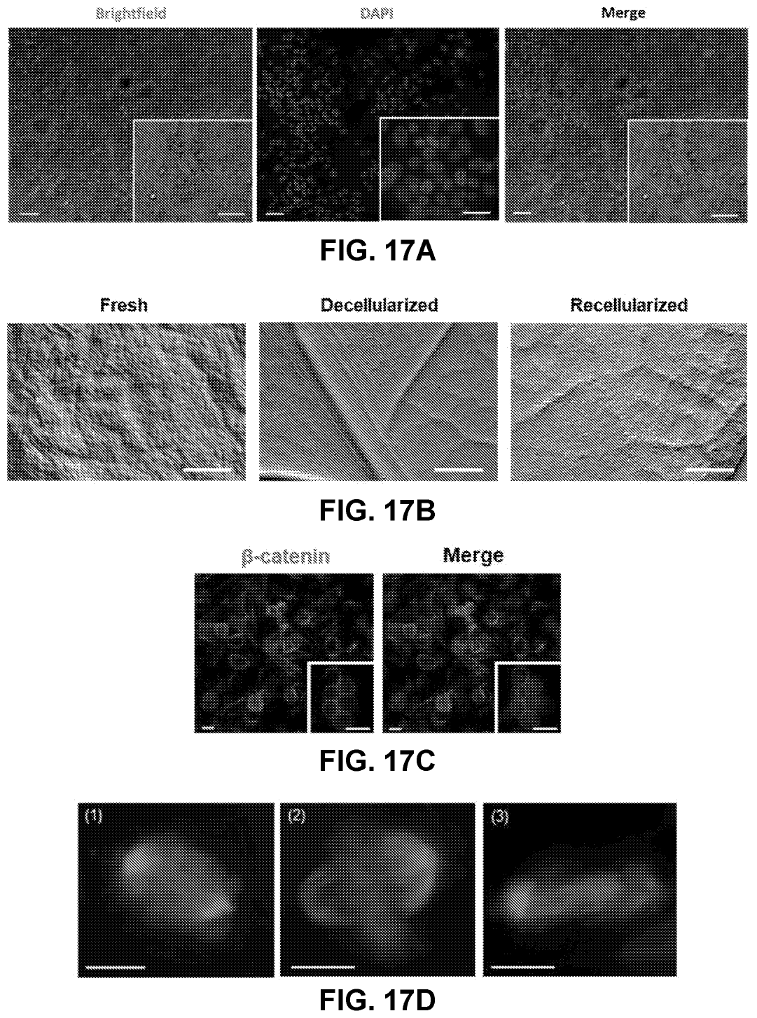

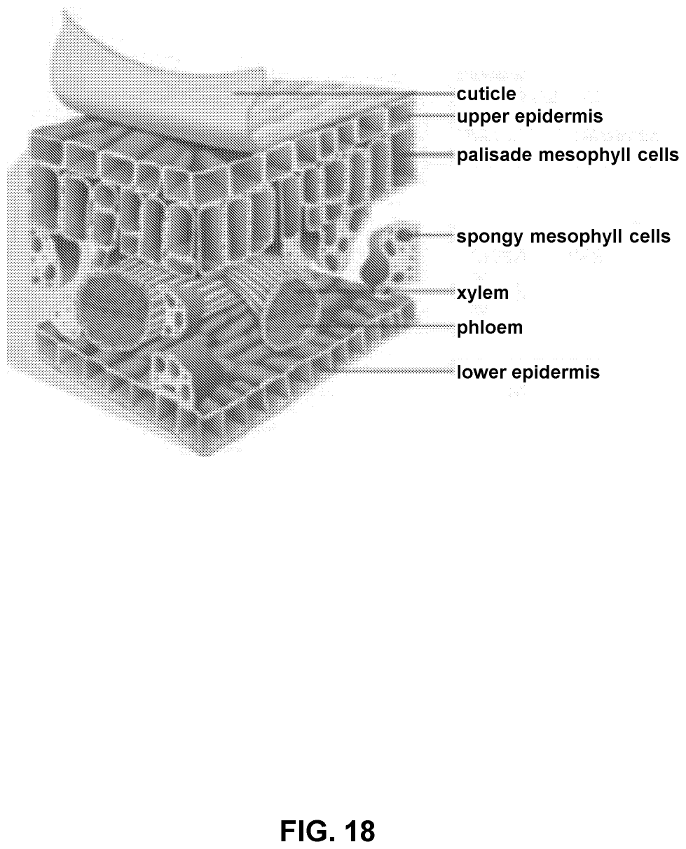

To assess this mechanical response, prostate cancer cell line (PC3) were first seeded on decellularized spinach leaves. Since mechanical and biochemical properties of the ECM regulate cell morphology, adhesion, proliferation, communication and tissue formation [15-17], leaf structures were first functionalized with a combination of collagen and fibronectin treatment in order to improve cell attachment and better mimic the presence of ECM. After 24 h incubation, fluorescence microscopy imaging showed that PC3 cells are present on the leaf surface (FIG. 17A). Larger scale optical 3D leaf topography and texture imaging was performed by using a GelSight tactile sensor [18]. The photometric results showed an apparent vasculature on decellularized leaf masked by presence of plant cells on fresh leaf and, in a lesser degree, by human cells on the recellularized scaffolds (FIG. 17B). In addition, fluorescence imaging showed that cells were able to create cell-cell junctions, such as adherens junctions, when visualized by the expression of β-catenin (FIG. 17C). Finally, well-formed mitotic spindles were visible in cells showing different phases of mitosis and thus demonstrating that cells are alive and able to divide on the leaf scaffold (FIG. 17D). All together, these results demonstrate that cells were able to attach onto the functionalized scaffold and were able to proliferate and form a tissue-like layer.

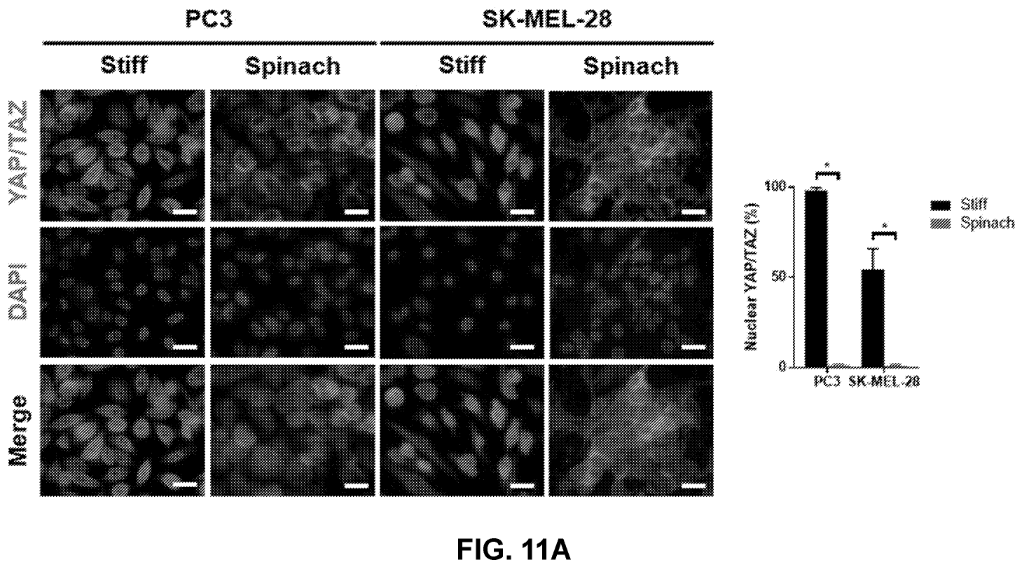

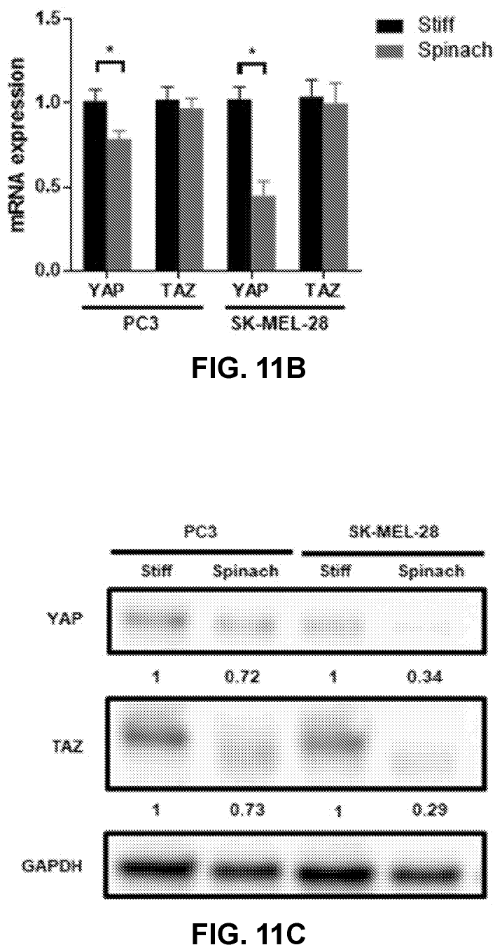

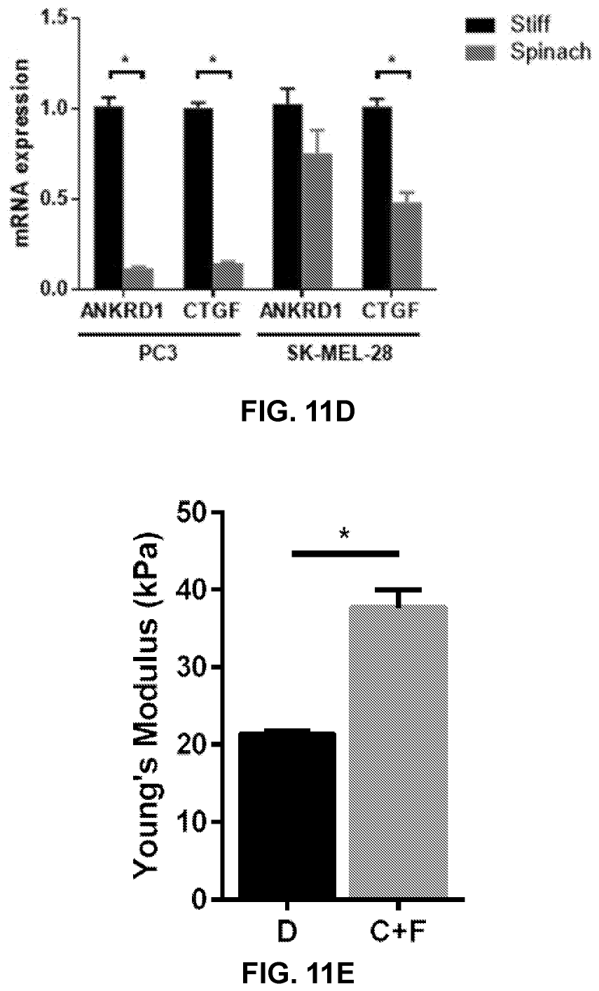

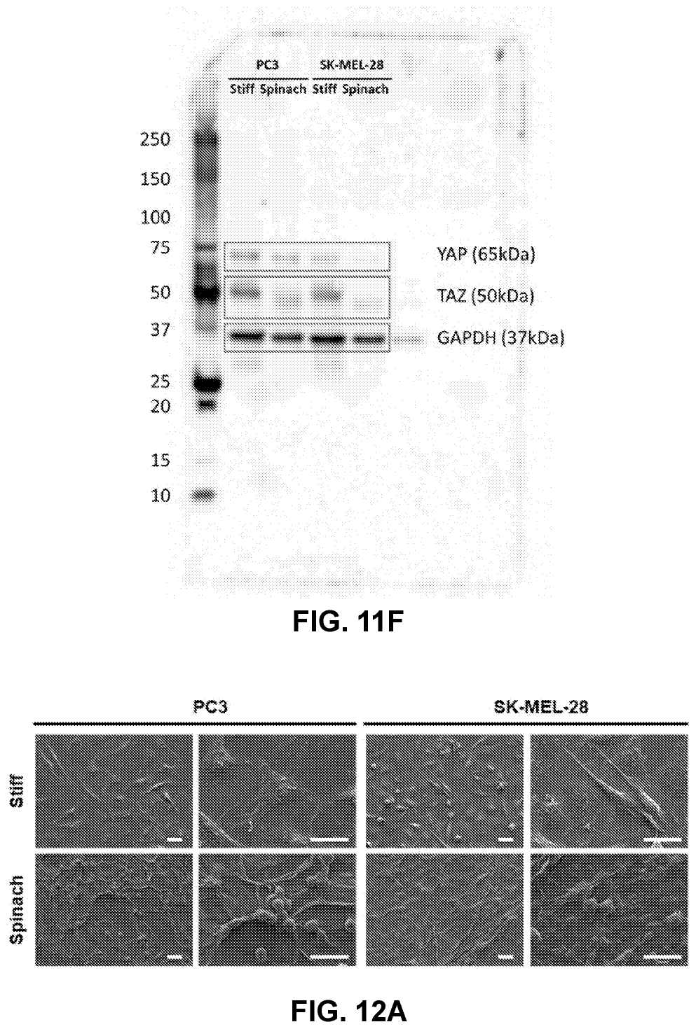

Since the recent identification of the Yorkie-homologues YAP (Yes-associated protein) and TAZ (transcriptional coactivator with PDZ-binding motif, also known as WWTR1) as nuclear sensors of mechanical signals exerted by substrate rigidity [19], a new model is emerging in which multiple types of mechanical inputs in a variety of cellular settings rely on the regulation of these two transcriptional regulators [20]. Therefore, endogenous YAP/TAZ subcellular localization was assessed by immunofluorescence in PC3 and SK-MEL-28 cells seeded on spinach leaf and stiff substrate. Results showed that YAP/TAZ were clearly nuclear on stiff substrate but became predominantly cytoplasmic on cells cultured on spinach leaf scaffold as shown in FIG. 11A (Immunofluorescence images of YAP/TAZ and nuclei (DAPI) in PC3 and SK-MEL-28 cells seeded on coverslip and spinach scaffold for 3 days. Scale bars=15 μm. Graphs indicate the percentage of cells with nuclear YAP/TAZ (n=3, *p<0.05; Student t-test)). In addition, mRNA expression of YAP was significantly decreased in PC3 and SK-MEL-28 cell lines (p=0.031 and p=0.0001 respectively) when cultured on spinach scaffolds while mRNA TAZ expression was not modified as shown in FIG. 11B (Quantitative RT-PCR analysis in PC3 and SK-MEL-28 cells to measure YAP and TAZ mRNA levels. Cells were grown on TCPS or spinach scaffold for 3 days. Data were normalized to expression on TCPS and indicated as mean±SEM (n=3, *p<0.05; Student t-test). YAP and TAZ proteins were also under-expressed in both cell lines when cultured on spinach scaffolds compared to stiff substrate as shown in FIG. 11C (Immunoblotting of YAP and TAZ in PC3 and SK-MEL-28 cells seeded on TCPS and spinach scaffolds). Finally, the measurement of the expression of two YAP/TAZ regulated genes, ANKRD1 and CTGF, showed that ANKRD1 was significantly downregulated in PC3 cells and CTGF was significantly downregulated in both cell lines seeded on leaf scaffold compared to stiff substrate as shown in FIG. 11D (Quantitative RT-PCR for YAP/TAZ target genes (CTGF and ANKRD1) in PC3 and SK-MEL-28 cells. Cells were grown on TCPS or spinach scaffold for 3 days. Data were normalized to expression on TCPS and indicated as mean±SEM (n=3, *p<0.05; Student t-test)). FIG. 11E shows Functionalization increases stiffness of decellularized scaffold. Comparison of the Young's modulus of decellularized (D) and functionalized (C+F) spinach leaf with collagen and fibronectin as measured by AFM. The graph shows the mean±SEM (n=3, *p<0.05; Student t-test). FIG. 11F shows original uncropped western blot image. Red rectangles correspond to the cropped sections shown in FIG. 11C. Altogether, these results suggested that YAP/TAZ pathway is not functional in cells cultured on leaf scaffold

Cell Culture on Leaf Scaffold Induces Cell Morphology Changes

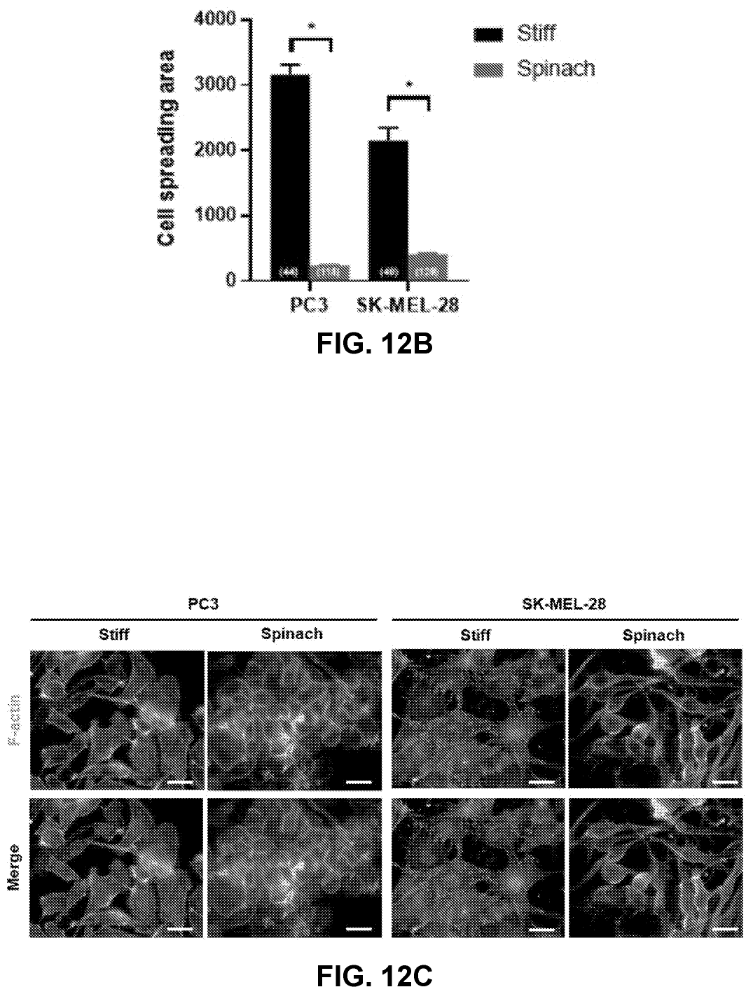

Because of low stiffness and YAP/TAZ inactivation, the tunability of cellular morphology of cells seeded on spinach scaffolds was investigated. Scanning electron microscopy revealed that both PC3 and SK-MEL-28 cells seeded on stiff substrate displayed an extended shape, with longer cell cilia, and a broader cell body compared to cells seeded on leaf scaffold that presented a round shape (FIG. 12A). The cell spreading area was quantified and shown to be 92.4 and 81% reduction for PC3 and SK-MEL-28 cells respectively when they are cultured on leaf scaffold compared to the stiff substrate (FIG. 12B). In addition, F-actin immunofluorescence confirmed this observation by revealing that cells maintain their round morphology with diffuse actin on leaf scaffold, whereas cells seeded on plastic substrate displayed a polygonal morphology with numerous F-actin stress fibers (FIG. 12C).

Proliferation Rate is Decreased for Cells Seeded on Leaf Scaffold

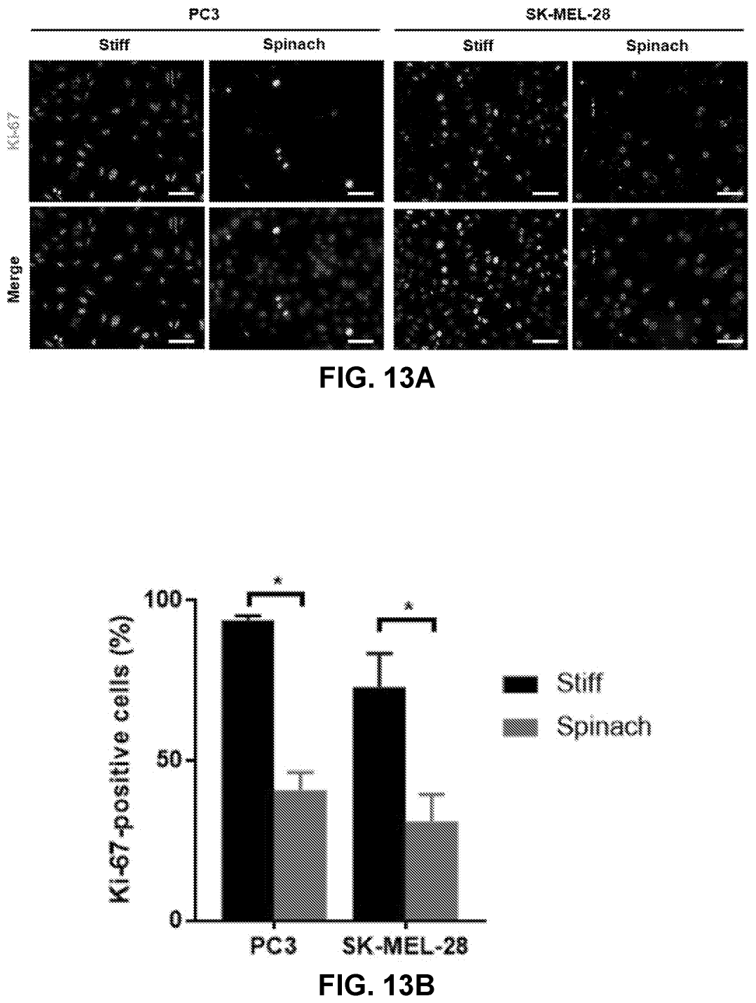

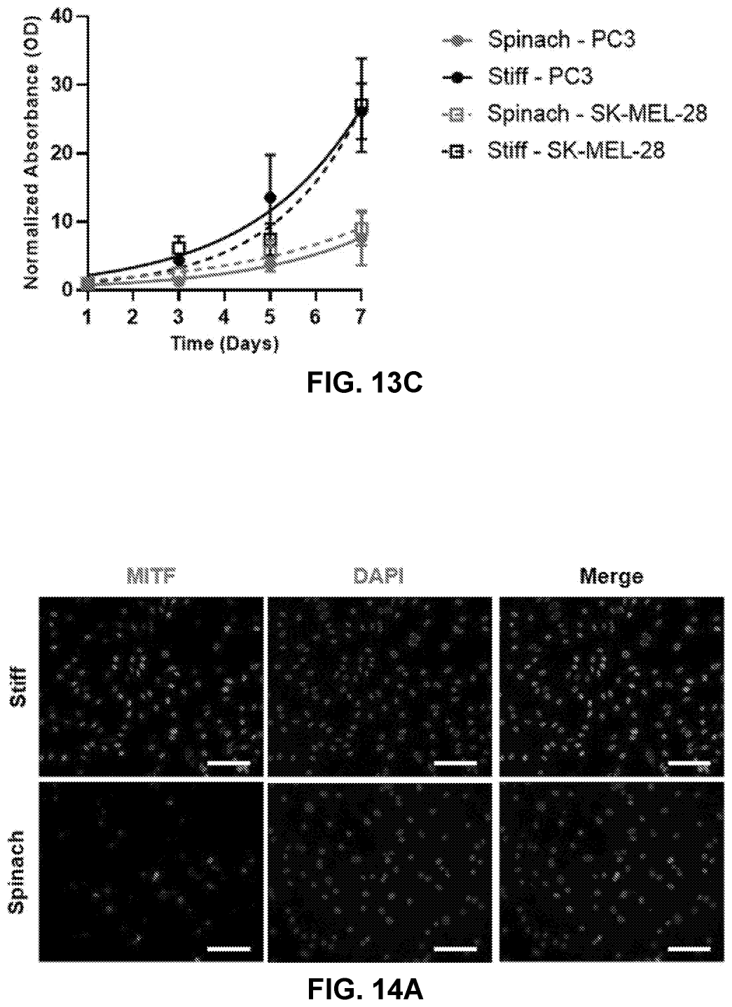

The proliferation activity of PC3 and SK-MEL-28 cells on stiff and leaf substrates was then assessed by measuring Ki-67 staining. Immunofluorescence showed that Ki-67 expression was upregulated in cells cultured for 72 hours on stiff substrate compared to leaf scaffold (FIG. 13A). The number of Ki-67-positive cells was significantly lower for PC3 and SK-MEL-28 cells (p<0.0001 and p=0.003, respectively) when they are cultured on spinach leaf scaffold (FIG. 13B), suggesting a potential higher proliferation rate of cells seeded on stiff substrate. To confirm this hypothesis, a modified MTT assay was then performed to assess proliferation of PC3 and SK-MEL-28 seeded on the leaf compared to a stiff substrate. Results showed that after 7 days, the normalized absorbance for PC3 and SK-MEL-28 cells grown on the stiff substrate is respectively 3.5-fold and 3-fold higher compared to the values for cells grown on the spinach leaf scaffold (FIG. 13C). Together these results showed that the cells growing on leaf scaffold have a lower proliferation rate than cells growing on a stiff substrate.

Cell Culture on Leaf Scaffold Changes Melanoma SK-MEL-28 Cells Phenotype

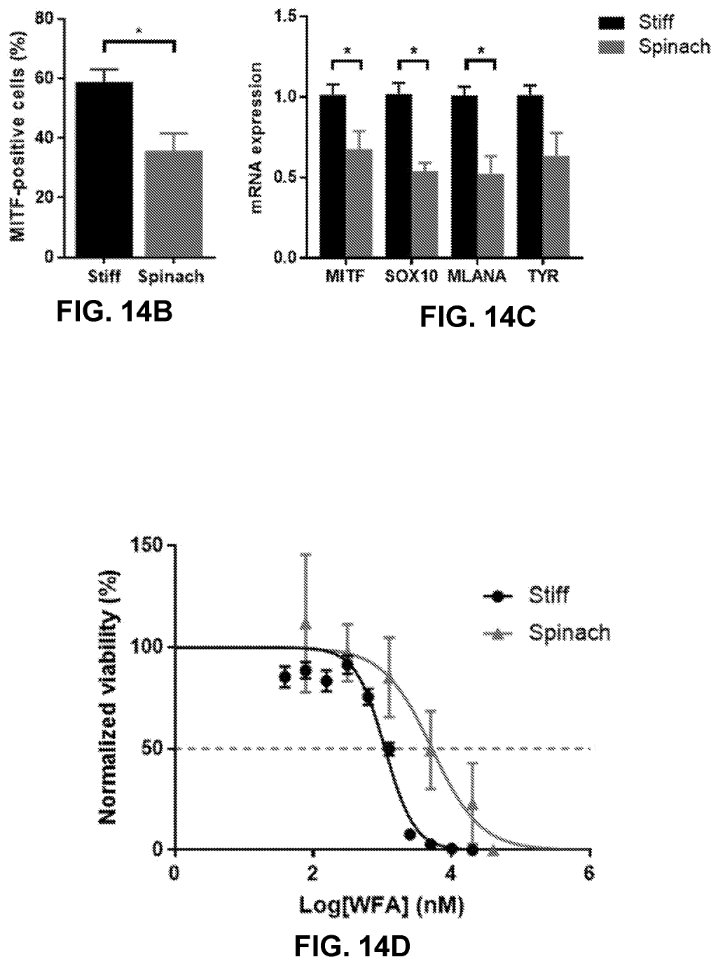

Microphthalmia-associated Transcription Factor (MITF) is a lineage-determining transcription factor critical for regulation of the melanocytic lineage during development and implicated as both a melanoma tumor suppressor and oncogene [21,22]. MITF is required for proliferation and has been identified as a factor prone to amplification [23,24]. Two phenotypically distinct populations of melanoma cells were described related to MITF levels: high-MITF population is associated with differentiation and proliferation, whereas low-MITF cells, although they proliferate slowly, are endowed with the invasive and EMT-like characteristics [25]. MITF is amplified in SKMel-28 cells [26] and, therefore, it was investigated whether its expression could be altered by the leaf scaffold. Immunofluorescence showed that MITF expression was upregulated in cells cultured on stiff substrate compared to leaf scaffold (FIG. 14A). The number of MITF-positive cells was significantly lower (p<0.016) for cells cultured on spinach leaf scaffold (FIG. 14B). Gene expression was then analyzed by qRT-PCR for MITF and three genes of the MITF-high expression signature (SOX10, MLANA and TYR) [27]. Results showed that MITF, and its target genes SOX10 and MLANA were significantly down-regulated when cells were cultured on decellularized spinach leaves compared to stiff substrate, suggesting that the leaf scaffold altered MITF and its associated pathways (FIG. 14C). Since MITF signature can impact drug response [28,29], response of SK-MEL-28 to withaferin A (WFA) was investigated. WFA is a natural compound from the withanolide family that induces apoptosis in human melanoma cell lines [30] and decreases uveal melanoma tumor growth in vivo [31]. Dose-response curves extracted from MTT assay showed that SK-MEL-28 cells seeded on stiff substrate were more sensitive to WFA (IC₅₀=1.1 μM) than cells on leaf scaffold (IC₅₀=5.2 μM).

Response of PC3 Cells to Radiation Differs Between Cells Seeded on Plastic and Leaf Substrate

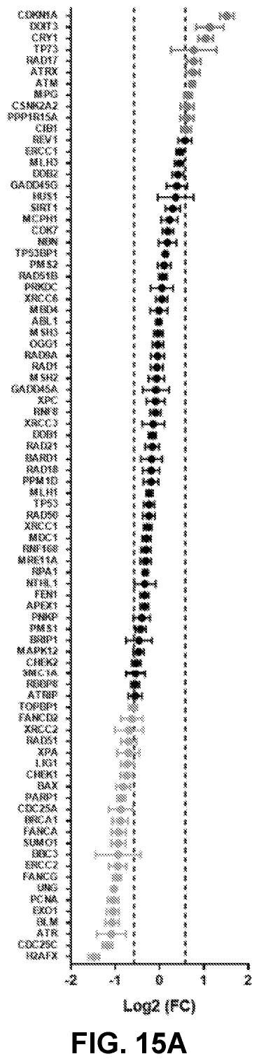

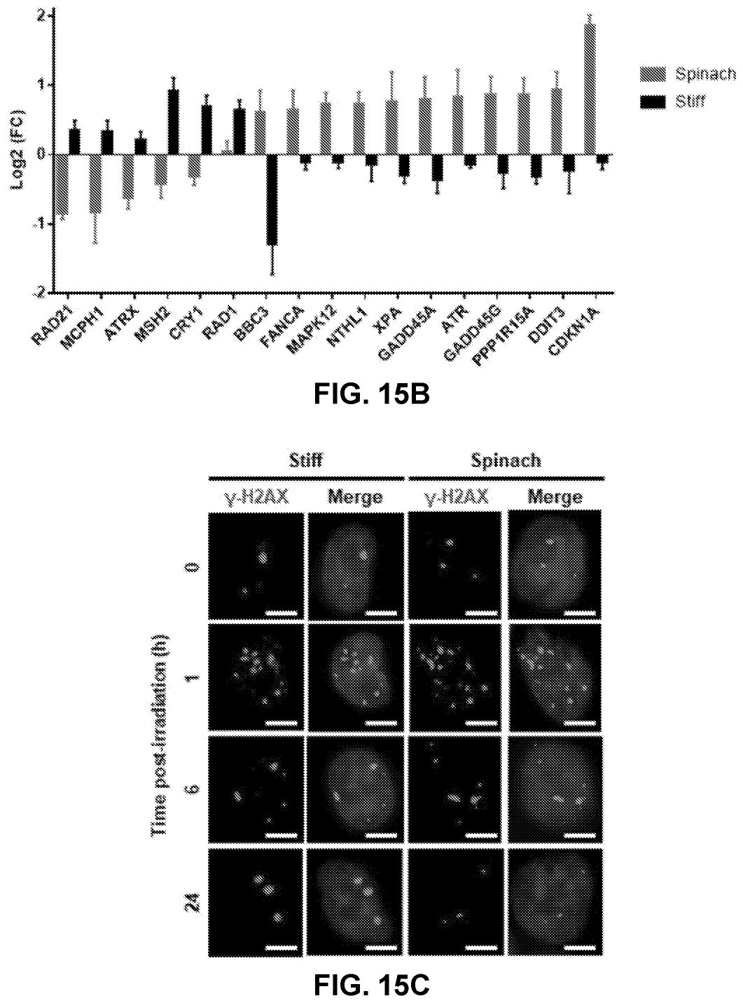

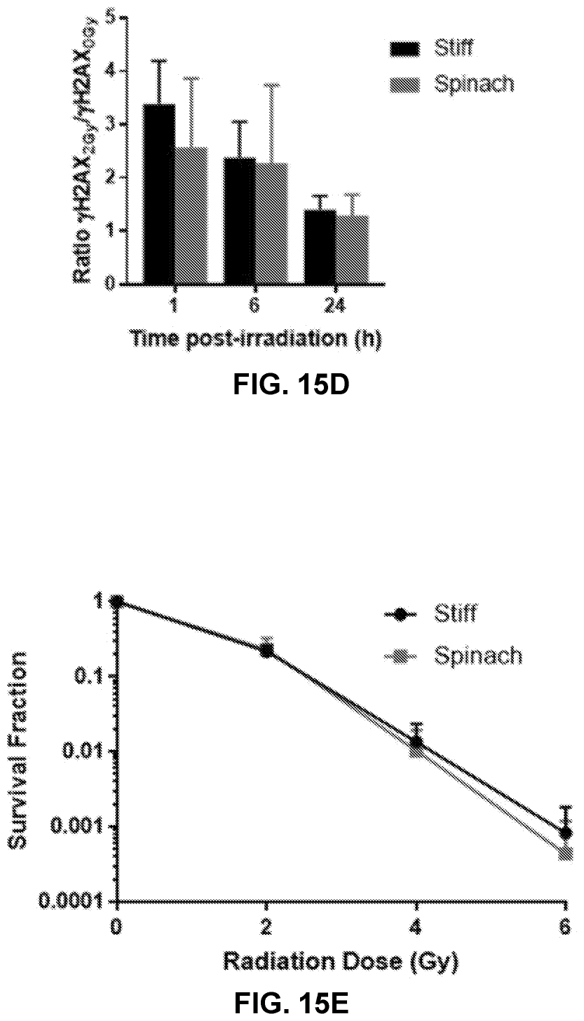

In addition to SK-MEL-28 drug response, PC3 cells were irradiated to assess whether cell culture on leaf scaffold could also modify radiation response. First, the expression of 84 genes involved in DNA damage signaling pathways were investigated in PC3 cells seeded for 3 days on leaf scaffold or TCPS. Results showed that 34 of 84 genes are differentially expressed (1.5<FC<0.66) between the two conditions, with 11 upregulated and 23 downregulated genes in PC3 cells cultured on spinach leaf scaffold compared to cells cultured in TCPS (FIG. 15A). Unexpectedly, the comparison of genes differentially expressed after 2 Gy-irradiation revealed that 11 genes (CDKN1A, DDIT3, PPP1R15A, GADD45G, ATR, GADD45A, XPA, NTHL1, MAPK12, FANCA and BBC3) are up-regulated and 6 genes (RAD1, CRY1, MSH2, ATRX, MCPH1 and RAD21) are down-regulated when cells were irradiated on spinach leaf scaffold (FIG. 15B). p53 signaling pathway is enriched by the upregulated genes while cell cycle is enriched by the downregulated genes, suggesting a potential switch in radiation-induced DNA damage signaling between leaf scaffold and stiff substrate. To investigate if radiosensitivity could be affected DNA double-strand breaks (DSBs) were assessed by monitoring the formation of γH2AX foci. As shown on FIG. 15C, the number of γH2AX foci increased 1 h after 2 Gy-irradiation compared to sham-irradiated in both cells seeded on spinach scaffold or stiff substrate. At 6 h post-irradiation, the number of foci started to decrease to return to normal level at 24 h. Interestingly, the kinetic of DBSs restoration is similar between cells seeded on spinach scaffold or stiff substrate suggesting that DNA damage is effectively repaired in both conditions (FIG. 15D). This was confirmed by clonogenic assay that showed that survival fraction of irradiated PC3 cells seeded on spinach leaf scaffold decreased following the same trend as cells seeded on stiff substrate demonstrating that PC3 radiosensitivity is similar between both conditions (FIG. 15E).

### REFERENCES ASSOCIATED WITH EXAMPLE 5

[1] Y. Fang, R. M. Eglen, Three-Dimensional Cell Cultures in Drug Discovery and Development, Slas Discov. 22 (2017) 456-472. https://doi.org/10.1177/1087057117696795.
[2] B. Ladoux, R.-M. Mege, Mechanobiology of collective cell behaviours, Nat. Rev. Mol. Cell Biol. 18 (2017) 743-757. https://doi.org/10.1038/nrm.2017.98.
[3] F. Broders-Bondon, T. H. Nguyen Ho-Bouldoires, M.-E. Fernandez-Sanchez, E. Farge, Mechanotransduction in tumor progression: The dark side of the force, J. Cell Biol. 217 (2018) 1571-1587. https://doi.org/10.1083/jcb.201701039.
[4] D. E. Discher, P. Janmey, Y. Wang, Tissue Cells Feel and Respond to the Stiffness of Their Substrate, Science. 310 (2005) 1139-1143. https://doi.org/10.1126/science.1116995.
[5] J. D. Humphrey, E. R. Dufresne, M. A. Schwartz, Mechanotransduction and extracellular matrix homeostasis, Nat. Rev. Mol. Cell Biol. 15 (2014) 802-812. https://doi.org/10.1038/nrm3896.
[6] H. Wolfenson, B. Yang, M. P. Sheetz, Steps in Mechanotransduction Pathways that Control Cell Morphology, Annu. Rev. Physiol. 81 (2019) 585-605. https://doi.org/10.1146/annurev-physiol-021317-121245.
[7] J. M. Northcott, I. S. Dean, J. K. Mouw, V. M. Weaver, Feeling Stress: The Mechanics of Cancer Progression and Aggression, Front. Cell Dev. Biol. 6 (2018). https://doi.org/10.3389/fcell.2018.00017.
[8] J. R. Gershlak, S. Hernandez, G. Fontana, L. R. Perreault, K. J. Hansen, S. A. Larson, B. Y. K. Binder, D. M. Dolivo, T. Yang, T. Dominko, M. W. Rolle, P. J. Weathers, F. Medina-Bolivar, C. L. Cramer, W. L. Murphy, G. R. Gaudette, Crossing kingdoms: Using decellularized plants as perfusable tissue engineering scaffolds, Biomaterials. 125 (2017) 13-22. https://doi.org/10.1016/j.biomaterials.2017.02.011.
[9] J. Read, G. D. Sanson, Characterizing sclerophylly: the mechanical properties of a diverse range of leaf types, New Phytol. 160 (2003) 81-99. https://doi.org/10.1046/j.1469-8137.2003.00855.x.
[10] S. Wang, L. Ren, Y. Liu, Z. Han, Y. Yang, Mechanical characteristics of typical plant leaves, J. Bionic Eng. 7 (2010) 294-300. https://doi.org/10.1016/S1672-6529(10)60253-3.
[11] K. Kitajima, S. J. Wright, J. W. Westbrook, Leaf cellulose density as the key determinant of inter- and intra-specific variation in leaf fracture toughness in a species-rich tropical forest, Interface Focus. 6 (2016) 20150100. https://doi.org/10.1098/rsfs.2015.0100.
[12] P. Hermanowicz, M. Sarna, K. Burda, H. Gabryś, AtomicJ: an open source software for analysis of force curves, Rev. Sci. Instrum. 85 (2014) 063703. https://doi.org/10.1063/1.4881683.
[13] K. J. Livak, T. D. Schmittgen, Analysis of relative gene expression data using real-time quantitative PCR and the 2(−Delta Delta C(T)) Method, Methods San Diego Calif. 25 (2001) 402-408. https://doi.org/10.1006/meth.2001.1262.
[14] P. M. Crapo, T. W. Gilbert, S. F. Badylak, An overview of tissue and whole organ decellularization processes, Biomaterials. 32 (2011) 3233-3243. https://doi.org/10.1016/j.biomaterials.2011.01.057.
[15] V. Vogel, Unraveling the Mechanobiology of Extracellular Matrix, Annu. Rev. Physiol. 80 (2018) 353-387. https://doi.org/10.1146/annurev-physiol-021317-121312.
[16] A. D. Theocharis, S. S. Skandalis, C. Gialeli, N. K. Karamanos, Extracellular matrix structure, Adv. Drug Deliv. Rev. 97 (2016) 4-27. https://doi.org/10.1016/j.addr.2015.11.001.
[17] J. K. Kular, S. Basu, R. I. Sharma, The extracellular matrix: Structure, composition, age-related differences, tools for analysis and applications for tissue engineering, J. Tissue Eng. 5 (2014) 2041731414557112. https://doi.org/10.1177/2041731414557112.
[18] M. K. Johnson, F. Cole, A. Raj, E. H. Adelson, Microgeometry capture using an elastomeric sensor, ACM Trans Graph. 30 (2011) 46-46. https://doi.org/10.1145/1964921.1964941.
[19] S. Dupont, L. Morsut, M. Aragona, E. Enzo, S. Giulitti, M. Cordenonsi, F. Zanconato, J. Le Digabel, M. Forcato, S. Bicciato, N. Elvassore, S. Piccolo, Role of YAP/TAZ in mechanotransduction, Nature. 474 (2011) 179-183. https://doi.org/10.1038/nature10137.
[20] A. Totaro, T. Panciera, S. Piccolo, YAP/TAZ upstream signals and downstream responses, Nat. Cell Biol. 20 (2018) 888-899. https://doi.org/10.1038/s41556-018-0142-z.
[21] L. A. Garraway, H. R. Widlund, M. A. Rubin, G. Getz, A. J. Berger, S. Ramaswamy, R. Beroukhim, D. A. Milner, S. R. Granter, J. Du, C. Lee, S. N. Wagner, C. Li, T. R. Golub, D. L. Rimm, M. L. Meyerson, D. E. Fisher, W. R. Sellers, Integrative genomic analyses identify MITF as a lineage survival oncogene amplified in malignant melanoma, Nature. 436 (2005) 117-122. https://doi.org/10.1038/nature03664.
[22] J. L. Simmons, C. J. Pierce, F. Al-Ejeh, G. M. Boyle, MITF and BRN2 contribute to metastatic growth after dissemination of melanoma, Sci. Rep. 7 (2017) 1-12. https://doi.org/10.1038/s41598-017-11366-y.
[23] S. Carreira, J. Goodall, L. Denat, M. Rodriguez, P. Nuciforo, K. S. Hoek, A. Testori, L. Larue, C. R. Goding, Mitf regulation of Dial controls melanoma proliferation and invasiveness, Genes Dev. 20 (2006) 3426-3439. https://doi.org/10.1101/gad.406406.
[24] K. S. Hoek, O. M. Eichhoff, N. C. Schlegel, U. Döbbeling, N. Kobert, L. Schaerer, S. Hemmi, R. Dummer, In vivo Switching of Human Melanoma Cells between Proliferative and Invasive States, Cancer Res. 68 (2008) 650-656. https://doi.org/10.1158/0008-5472.CAN-07-2491.
[25] K. Vlçkova, J. Vachtenheim, J. Réda, P. Horák, L. Ondrušová, Inducibly decreased MITF levels do not affect proliferation and phenotype switching but reduce differentiation of melanoma cells, J. Cell. Mol. Med. 22 (2018) 2240-2251. https://doi.org/10.1111/jcmm.13506.
[26] C. Wellbrock, S. Rana, H. Paterson, H. Pickersgill, T. Brummelkamp, R. Marais, Oncogenic BRAF Regulates Melanoma Proliferation through the Lineage Specific Factor MITF, PLOS ONE. 3 (2008) e2734. https://doi.org/10.1371/journal.pone.0002734.
[27] M. Ennen, C. Keime, G. Gambi, A. Kieny, S. Coassolo, C. Thibault-Carpentier, F. Margerin-Schaller, G. Davidson, C. Vagne, D. Lipsker, I. Davidson, MITF-High and MITF-Low Cells and a Novel Subpopulation Expressing Genes of Both Cell States Contribute to Intra- and Intertumoral Heterogeneity of Primary Melanoma, Clin. Cancer Res. Off. J. Am. Assoc. Cancer Res. 23 (2017) 7097-7107. https://doi.org/10.1158/1078-0432.CCR-17-0010.
[28] S. Aida, Y. Sonobe, H. Tanimura, N. Oikawa, M. Yuhki, H. Sakamoto, T. Mizuno, MITF suppression improves the sensitivity of melanoma cells to a BRAF inhibitor, Cancer Lett. 409 (2017) 116-124. https://doi.org/10.1016/j.canlet.2017.09.008.
[29] J. Müller, O. Krijgsman, J. Tsoi, L. Robert, W. Hugo, C. Song, X. Kong, P. A. Possik, P. D. M. Cornelissen-Steijger, M. H. G. Foppen, K. Kemper, C. R. Goding, U. McDermott, C. Blank, J. Haanen, T. G. Graeber, A. Ribas, R. S. Lo, D. S. Peeper, Low MITF/AXL ratio predicts early resistance to multiple targeted drugs in melanoma, Nat. Commun. 5 (2014) 1-15. https://doi.org/10.1038/ncomms6712.
[30] E. Mayola, C. Gallerne, D. D. Esposti, C. Martel, S. Pervaiz, L. Larue, B. Debuire, A. Lemoine, C. Brenner, C. Lemaire, Withaferin A induces apoptosis in human melanoma cells through generation of reactive oxygen species and down-regulation of Bcl-2, Apoptosis Int. J. Program. Cell Death. 16 (2011) 1014-1027. https://doi.org/10.1007/s10495-011-0625-x.
[31] A. K. Samadi, S. M. Cohen, R. Mukerji, V. Chaguturu, X. Zhang, B. N. Timmermann, M. S. Cohen, E. A. Person, Natural withanolide withaferin A induces apoptosis in uveal melanoma cells by suppression of Akt and c-MET activation, Tumour Biol. J. Int. Soc. Oncodevelopmental Biol. Med. 33 (2012) 1179-1189. https://doi.org/10.1007/s13277-012-0363-x.
[32] D. T. Butcher, T. Alliston, V. M. Weaver, A tense situation: forcing tumour progression, Nat. Rev. Cancer. 9 (2009) 108-122. https://doi.org/10.1038/nrc2544.
[33] F. Liu, D. Lagares, K. M. Choi, L. Stopfer, A. Marinković, V. Vrbanac, C. K. Probst, S. E. Hiemer, T. H. Sisson, J. C. Horowitz, I. O. Rosas, L. E. Fredenburgh, C. Feghali-Bostwick, X. Varelas, A. M. Tager, D. J. Tschumperlin, Mechanosignaling through YAP and TAZ drives fibroblast activation and fibrosis, Am. J. Physiol. Lung Cell. Mol. Physiol. 308 (2015) L344-357. https://doi.org/10.1152/ajplung.00300.2014.
[34] S. E. Cross, Y.-S. Jin, J. Rao, J. K. Gimzewski, Nanomechanical analysis of cells from cancer patients, Nat. Nanotechnol. 2 (2007) 780-783. https://doi.org/10.1038/nnano.2007.388.
[35] B. D. James, W. N. Ruddick, S. E. Vasisth, K. Dulany, S. Sulekar, A. Porras, A. Marañon, J. C. Nino, J. B. Allen, Palm readings: Manicaria saccifera palm fibers are biocompatible textiles with low immunogenicity, Mater. Sci. Eng. C. 108 (2020) 110484. https://doi.org/10.1016/j.msec.2019.110484.
[36] K. A. Jansen, D. M. Donato, H. E. Balcioglu, T. Schmidt, E. H. J. Danen, G. H. Koenderink, A guide to mechanobiology: Where biology and physics meet, Biochim. Biophys. Acta BBA—Mol. Cell Res. 1853 (2015) 3043-3052. https://doi.org/10.1016/j.bbamcr.2015.05.007.
[37] Y. Yang, K. Wang, X. Gu, K. W. Leong, Biophysical Regulation of Cell Behavior—Cross Talk between Substrate Stiffness and Nanotopography, Engineering. 3 (2017) 36-54. https://doi.org/10.1016/J.ENG.2017.01.014.
[38] H. Salmon, K. Franciszkiewicz, D. Damotte, M.-C. Dieu-Nosjean, P. Validire, A. Trautmann, F. Mami-Chouaib, E. Donnadieu, Matrix architecture defines the preferential localization and migration of T cells into the stroma of human lung tumors, J. Clin. Invest. 122 (2012) 899-910. https://doi.org/10.1172/JCI45817.
[39] K. R. Levental, H. Yu, L. Kass, J. N. Lakins, M. Egeblad, J. T. Erler, S. F. T. Fong, K. Csiszar, A. Giaccia, W. Weninger, M. Yamauchi, D. L. Gasser, V. M. Weaver, Matrix crosslinking forces tumor progression by enhancing integrin signaling, Cell. 139 (2009) 891-906. https://doi.org/10.1016/j.cell.2009.10.027.
[40] G. Fontana, J. Gershlak, M. Adamski, J.-S. Lee, S. Matsumoto, H. D. Le, B. Binder, J. Wirth, G. Gaudette, W. L. Murphy, Biofunctionalized Plants as Diverse Biomaterials for Human Cell Culture, Adv. Healthc. Mater. 6 (2017). https://doi.org/10.1002/adhm.201601225.
[41] S. Dikici, F. Claeyssens, S. MacNeil, Decellularised baby spinach leaves and their potential use in tissue engineering applications: Studying and promoting neovascularisation, J. Biomater. Appl. 34 (2019) 546-559. https://doi.org/10.1177/0885328219863115.
[42] J. Xie, D. Zhang, C. Zhou, Q. Yuan, L. Ye, X. Zhou, Substrate elasticity regulates adipose-derived stromal cell differentiation towards osteogenesis and adipogenesis through β-catenin transduction, Acta Biomater. 79 (2018) 83-95. https://doi.org/10.1016/j.actbio.2018.08.018.
[43] Q. Zhang, Y. Yu, H. Zhao, The effect of matrix stiffness on biomechanical properties of chondrocytes, Acta Biochim. Biophys. Sin. 48 (2016) 958-965. https://doi.org/10.1093/abbs/gmw087.
[44] K.-I. Wada, K. Itoga, T. Okano, S. Yonemura, H. Sasaki, Hippo pathway regulation by cell morphology and stress fibers, Dev. Camb. Engl. 138 (2011) 3907-3914. https://doi.org/10.1242/dev.070987.
[45] S. Piccolo, S. Dupont, M. Cordenonsi, The Biology of YAP/TAZ: Hippo Signaling and Beyond, Physiol. Rev. 94 (2014) 1287-1312. https://doi.org/10.1152/physrev.00005.2014.
[46] F. Zanconato, M. Forcato, G. Battilana, L. Azzolin, E. Quaranta, B. Bodega, A. Rosato, S. Bicciato, M. Cordenonsi, S. Piccolo, Genome-wide association between YAP/TAZ/TEAD and AP-1 at enhancers drives oncogenic growth, Nat. Cell Biol. 17 (2015) 1218-1227. https://doi.org/10.1038/ncb3216.
[47] F. Zanconato, M. Cordenonsi, S. Piccolo, YAP and TAZ: a signalling hub of the tumour microenvironment, Nat. Rev. Cancer. 19 (2019) 454-464. https://doi.org/10.1038/s41568-019-0168-y.
[48] B. Zhao, X. Wei, W. Li, R. S. Udan, Q. Yang, J. Kim, J. Xie, T. Ikenoue, J. Yu, L. Li, P. Zheng, K. Ye, A. Chinnaiyan, G. Halder, Z.-C. Lai, K.-L. Guan, Inactivation of YAP oncoprotein by the Hippo pathway is involved in cell contact inhibition and tissue growth control, Genes Dev. 21 (2007) 2747-2761. https://doi.org/10.1101/gad.1602907.
[49] Z. Miskolczi, M. P. Smith, E. J. Rowling, J. Ferguson, J. Barriuso, C. Wellbrock, Collagen abundance controls melanoma phenotypes through lineage-specific microenvironment sensing, Oncogene. 37 (2018) 3166-3182. https://doi.org/10.1038/s41388-018-0209-0.
[50] S. Carreira, J. Goodall, I. Aksan, S. A. La Rocca, M.-D. Galibert, L. Denat, L. Larue, C. R. Goding, Mitf cooperates with Rb1 and activates p21Cip1 expression to regulate cell cycle progression, Nature. 433 (2005) 764-769. https://doi.org/10.1038/nature03269.
[51] A. E. Loercher, E. M. H. Tank, R. B. Delston, J. W. Harbour, MITF links differentiation with cell cycle arrest in melanocytes by transcriptional activation of INK4A, J. Cell Biol. 168 (2005) 35-40. https://doi.org/10.1083/jcb.200410115.
[52] G. Pathria, B. Garg, V. Borgdorff, K. Garg, C. Wagner, G. Superti-Furga, S. N. Wagner, Overcoming MITF-conferred drug resistance through dual AURKA/MAPK targeting in human melanoma cells, Cell Death Dis. 7 (2016) e2135. https://doi.org/10.1038/cddis.2015.369.
[53] J. L. Leight, A. P. Drain, V. M. Weaver, Extracellular Matrix Remodeling and Stiffening Modulate Tumor Phenotype and Treatment Response, Annu. Rev. Cancer Biol. 1 (2017) 313-334. https://doi.org/10.1146/annurev-cancerbio-050216-034431.
[54] T. Stylianopoulos, L. L. Munn, R. K. Jain, Reengineering the Physical Microenvironment of Tumors to Improve Drug Delivery and Efficacy: From Mathematical Modeling to Bench to Bedside, Trends Cancer. 4 (2018) 292-319. https://doi.org/10.1016/j.trecan.2018.02.005.
[55] A. Singh, I. Brito, J. Lammerding, Beyond Tissue Stiffness and Bioadhesivity: Advanced Biomaterials to Model Tumor Microenvironments and Drug Resistance, Trends Cancer. 4 (2018) 281-291. https://doi.org/10.1016/j.trecan.2018.01.008.
[56] A. Fernandez-L, M. Squatrito, P. Northcott, A. Awan, E. C. Holland, M. D. Taylor, Z. Nahlé, A. M. Kenney, Oncogenic YAP promotes radioresistance and genomic instability in medulloblastoma through IGF2-mediated Akt activation, Oncogene. 31 (2012) 1923-1937. https://doi.org/10.1038/onc.2011.379.
[57] H. Cheng, Z. Zhang, R. Rodriguez-Barrueco, A. Borczuk, H. Liu, J. Yu, J. M. Silva, S. K. Cheng, R. Perez-Soler, B. Halmos, Functional genomics screen identifies YAP1 as a key determinant to enhance treatment sensitivity in lung cancer cells, Oncotarget. 7 (2016) 28976-28988. https://doi.org/10.18632/oncotarget.6721.
[58] J. Y. Lee, J. K. Chang, A. A. Dominguez, H. Lee, S. Nam, J. Chang, S. Varma, L. S. Qi, R. B. West, O. Chaudhuri, YAP-independent mechanotransduction drives breast cancer progression, Nat. Commun. 10 (2019) 1-9. https://doi.org/10.1038/s41467-019-09755-0.
[59] D. J. Modulevsky, C. Lefebvre, K. Haase, Z. Al-Rekabi, A. E. Pelling, Apple Derived Cellulose Scaffolds for 3D Mammalian Cell Culture, PLOS ONE. 9 (2014) e97835. https://doi.org/10.1371/journal.pone.0097835.
[60] S. R. Caliari, J. A. Burdick, A Practical Guide to Hydrogels for Cell Culture, Nat. Methods. 13 (2016) 405-414. https://doi.org/10.1038/nmeth.3839.

### Example 6: Skin-On-Leaf Multilayered Vascularized Biological System to Study LET Radiation Effects

Research has demonstrated that the biological response to space radiation is unique due to a non-homogeneous, multi-energetic dose distribution (1). While the effect of such high-linear energy transfer (LET) radiation might be mitigated with effective shielding, an exposed human would be expected to receive skin doses 5-10-times higher than those experienced by internal organs during deep-space missions, because of its superficial location and susceptibility to absorption in the low energy spectra of protons and nuclei (2). Unfortunately, although the description of radiation-induced skin damage is relatively well documented, the late toxicity profiles and the underlying mechanisms are still poorly understood and prevent the development of efficient countermeasure approaches. The skin is a complex organ, especially from a biomechanical perspective, and there is a need to use more developed in vitro human models to overcome these challenges. Accordingly, decellularized plant leaves to provide three-dimensional (3D) biocompatible scaffold and create a “skin-on-leaf” multilayered vascularized biological system reproducing the different skin layers according to their mechanical properties and cellular components. The unique feature of these scaffolds allows vascularization of the model, thus interrogating the role of endothelial cells in radiation-induced skin injuries.

The “skin-on-leaf” may be exposed to X-ray (as reference) and high LET neutrons. The response of keratinocytes and dermal fibroblasts in the different layers as well as endothelial cells may be assessed.

The radiation response may be assessed with a rapamycin drug analog (everolimus) injected in the dermis vascular system. Testing of drug efficacy against high LET radiation may be conducted using neutron irradiation as well as acute and protracted galactic cosmic radiation (GCR).

Needle-generated stress may be induced on the skin-on-leaf and mechanotransduction signals may be investigated to assess whether such contacts can improve skin and stem cells resistance to space-relevant radiation.

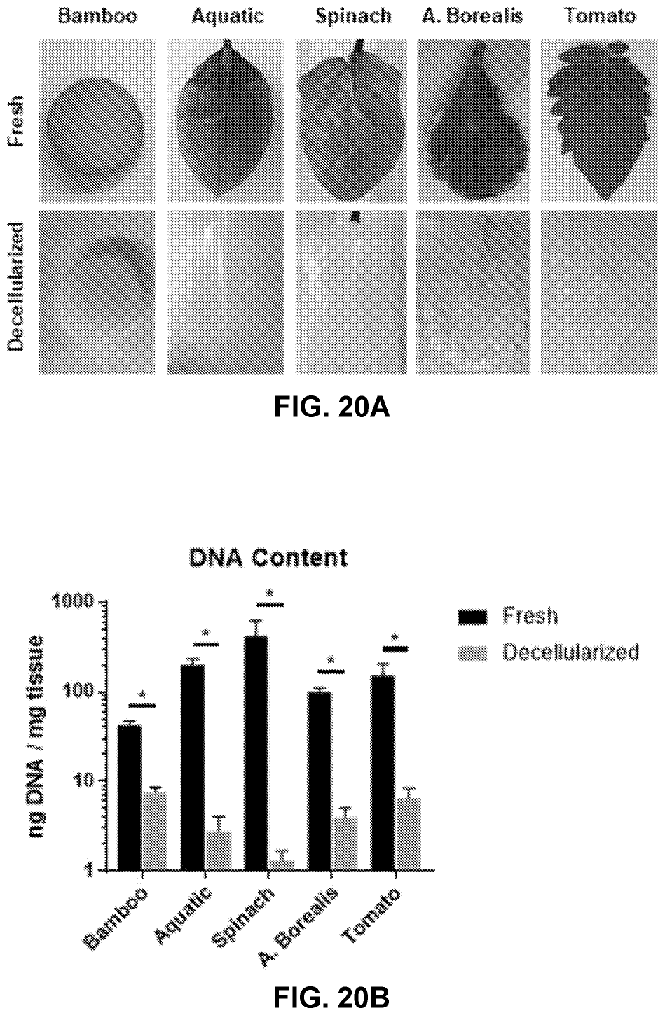

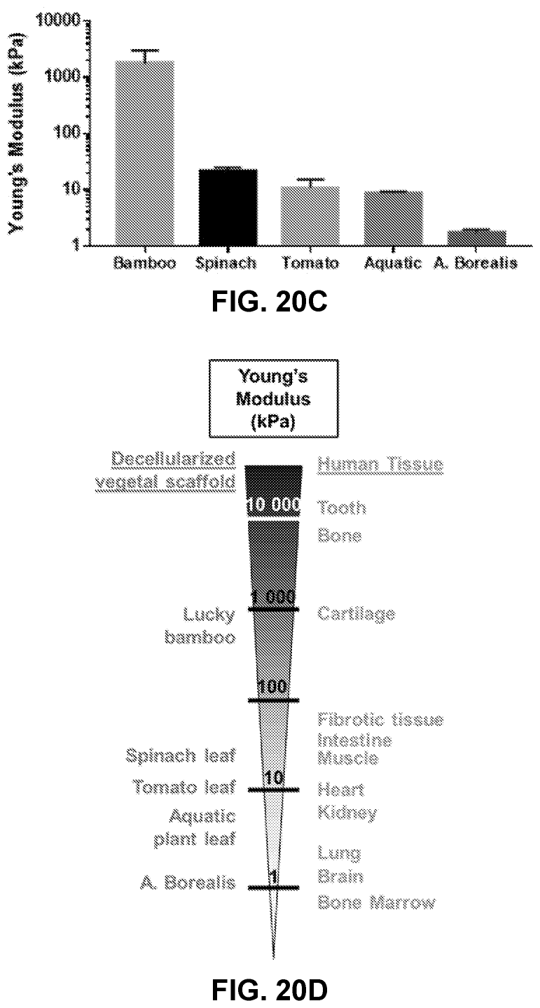

Different plant materials were decellularized to assess their stiffness and evaluate if they could match the range of human tissue stiffness. Plants, including baby spinach leaves, hybrid cherry tomato plant, aquatic plant, A. borealis and lucky bamboo, were decellularized by serial chemical treatment. After 7 days, all vegetal structures lost chlorophyll and appeared fully translucent (FIG. 20A). Decellularized leaves contained significantly less DNA (FIG. 20B) than fresh leaves, suggesting that plant material have been successfully removed from the native structure. Using AFM-based force mapping, we measured stiffness of decellularized scaffolds and found that they displayed a broad range of stiffness, with Young's modulus (YM) from 1.7±0.3 (A. borealis) to 1767±1260 kPa (bamboo) (FIG. 20C), thus matching stiffness of main human tissues (FIG. 20D) (14-18).

Recellularization of Decellularized Plant-Based Scaffolds and “Skin-On-Leaf” Model

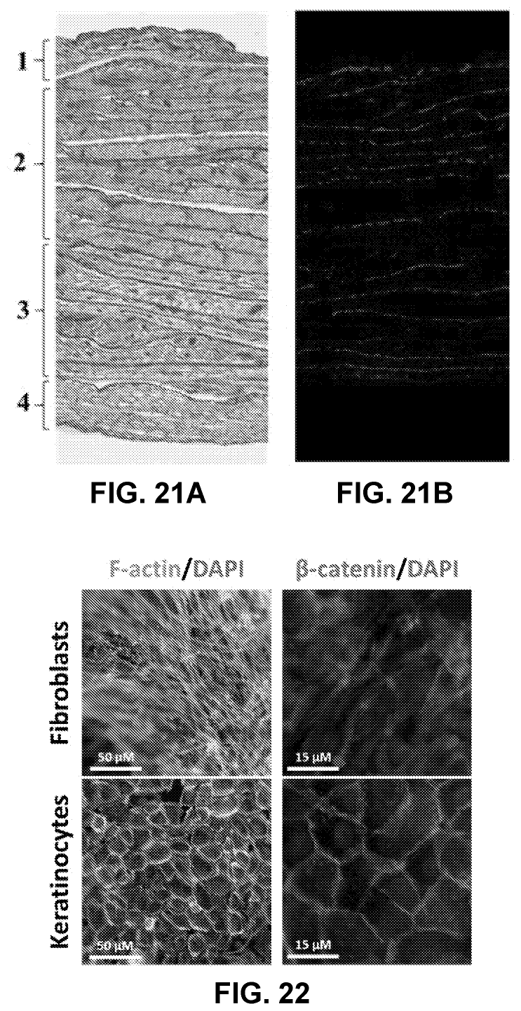

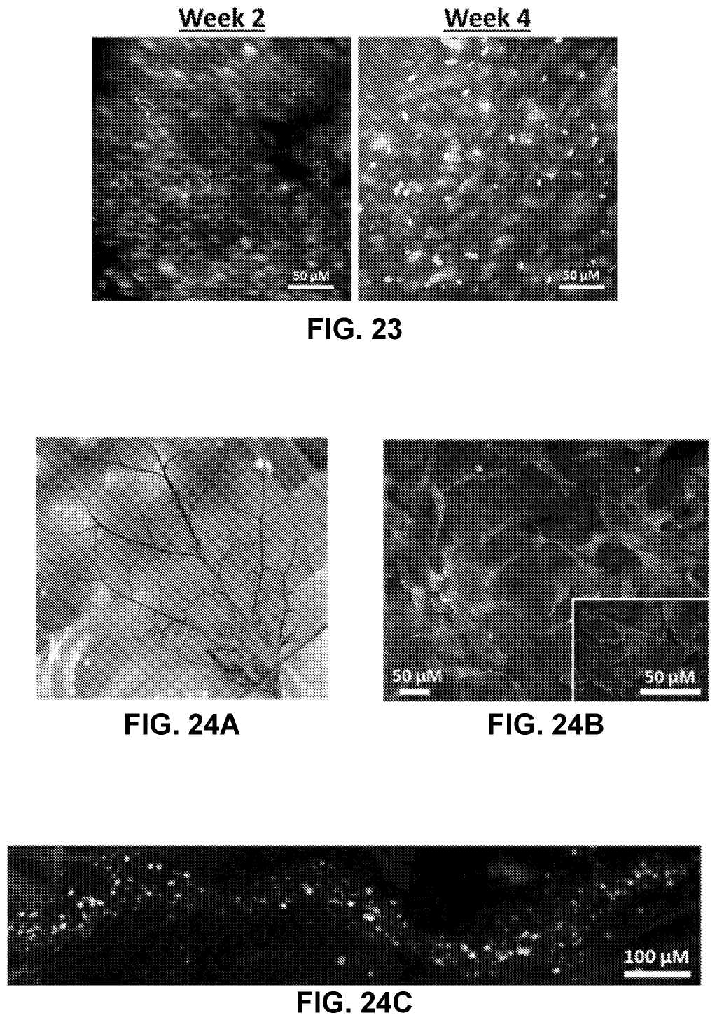

From a mechanical point of view, human skin appears as a layered composite containing the stiff thin cover layer presented by the stratum corneum (YM=100 kPa), below which are the more compliant layers of viable epidermis (YM=25 kPa) and dermis (YM=75 kPa) and further below the much softer adjacent layer of subcutaneous white adipose tissue (YM=8 kPa) (19,20). Although the correlation of YM with intramural and extraneous factors such as Langer's lines, skin's thickness, ageing and hydration make difficult to consider these numbers as universal values, they show the high anisotropy of the skin and suggest that its mechanics is complex. Using these bulk YM values, the multi-layer (stratified) skin model was created by using cellulose scaffolds from decellularized spinach to form the stiffest, top most layer (stratum corneum) of the epidermis, tomato leaves to form the epidermis, spinach leaves to form the dermis and A. borealis to form the softest subcutaneous layer (FIGS. 21A-21B). In this model, after functionalization of scaffolds with collagen and fibronectin, human normal epidermal keratinocytes were seeded on tomato leaves and human normal fibroblasts on spinach leaves to provide the cellular components of epidermis and dermis respectively. Fluorescence microscopy imaging confirmed that cells were present on the leaf surface (F-actin) and able to create tight junctions, as visualized by the expression of β-catenin (FIG. 22). All together, these results demonstrate that cells were able to attach onto the functionalized leaf scaffolds and were able to proliferate and form a tissue-like layer. In addition, these cells can survive up to four weeks in the system (FIG. 23).

Vascularization of Plant-Based Decellularized Scaffolds and Role of Endothelial Cells in Skin Cells Response to IR

In addition to their mechanical properties, one of the main advantages of the plant-based scaffolds is their natural vasculature. Once decellularized, the leaf veins remain and offer a network nearly identical to animal capillaries systems (FIG. 24A). In a similar manner to keratinocyte and fibroblast cells, human endothelial cells (HUVECs) can also be attached on leaf scaffold where they are able to connect and form cell junction (FIG. 24B). Interestingly, endothelial cells may be seeded within the veins to recreate a vascular network (FIG. 24C). Radiation damage to skin includes erythema, pigmentation, and dry and moist desquamation in the early phase (<4 weeks) and atrophy and fibrosis (or necrosis) in the later phase (>6 weeks) (22). However, radiation also damages the vasculature of the dermis, progressively changing superficial blood vessel structure, and often leading to a prominent, enlarged, and tortuous microvasculature near the skin surface (telangiectasia) (23). As radiation-damaged endothelial cells are not replaced within the tuft after irradiation (24,25), microvessel ends progressively retract and surviving endothelial cells enlarge to cover the affected area. This results in partial loss of adjacent basal and dermal cells. At some points in time, vessel continuity is broken by continued endothelial cell loss, manifesting telangiectasia under a thinned epidermis as an area of reddish discoloration with many dilated blood vessels.

Mechanotransduction Pathways Activity of Cells Seeded on Plant-Based Scaffolds

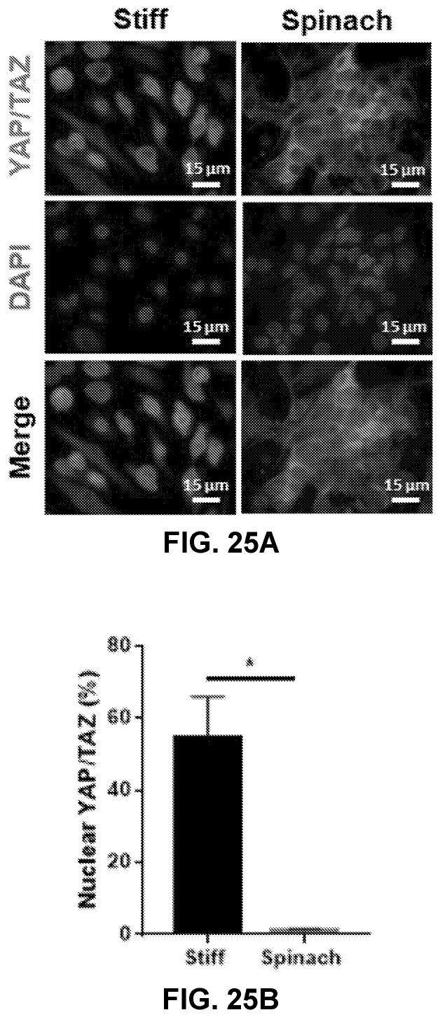

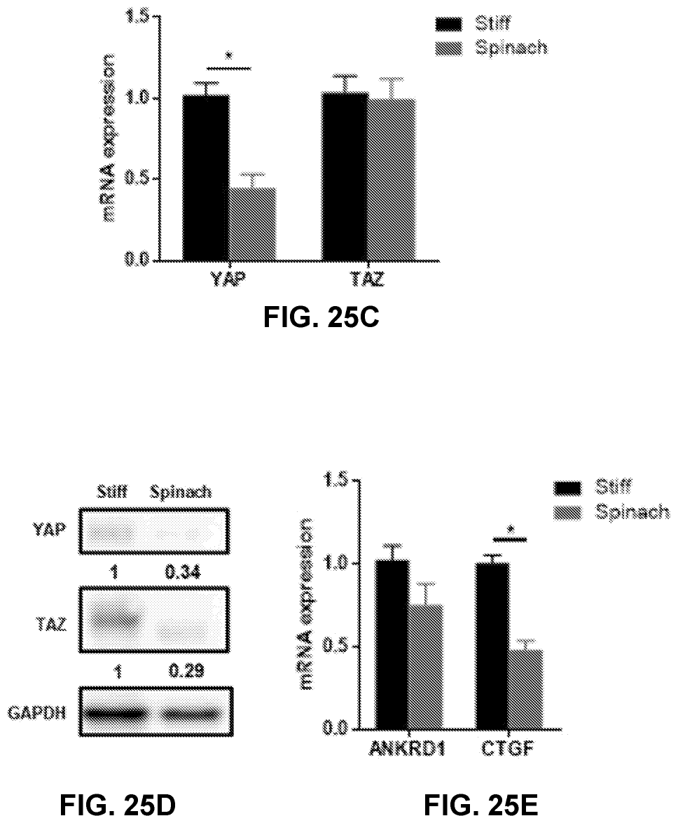

Yorkie-homologues YAP (Yes-associated protein) and TAZ (transcriptional coactivator with PDZ-binding motif, also known as WWTR1) pathways, were evaluated as a master regulator of mechanotransduction response. Cells were seeded on vegetal scaffolds and compared to stiff substrates. Endogenous YAP/TAZ subcellular localization was assessed via immunofluorescence of melanoma SK-MEL-28 cells seeded on spinach leaf and stiff substrate. Results showed that YAP/TAZ were clearly nuclear on stiff substrate but became predominantly cytoplasmic on cells cultured on spinach leaf scaffold (FIGS. 25A-25B). In addition, mRNA expression of YAP was significantly decreased in cells (p=0.0001) when cultured on spinach scaffolds while mRNA TAZ expression was not altered (FIG. 25C). YAP and TAZ proteins were also under-expressed in both cell lines when cultured on spinach scaffolds compared to stiff substrate (FIG. 25D). Finally, the measurement of the expression of two YAP/TAZ regulated genes, ANKRD1 and CTGF, showed that both are downregulated in cells are seeded on leaf scaffold (FIG. 25E). Thus, YAP/TAZ mechanotransduction pathways of cells seeded on leaf model is downregulated compared to standard cell culture. Proliferation activity is decreased when YAP is cytoplasmic and highlight the importance of having a multi-layer model to recapitulate the profile of the mechanotransduction pathways of the skin.

Radiation Response of Human Cells Seeded on Plant-Based Decellularized Scaffolds

The radiation response of prostate cancer cells (PC3) seeded on plant-based scaffolds was compared to conventional cell culture substrates such as TCPS flasks or glass coverslips. The expression of 84 genes involved in DNA damage signaling pathways was investigated. Results showed that 34 of 84 genes are differentially expressed (1.5<FC<0.66) between the two conditions, with 11 upregulated and 23 downregulated genes in PC3 cells cultured on spinach leaf scaffold compared to cells cultured in TCPS (FIG. 15A). Interestingly, the comparison of genes differentially expressed after 2 Gy-irradiation revealed that 11 genes (CDKN1A, DDIT3, PPP1R15A, GADD45G, ATR, GADD45A, XPA, NTHL1, MAPK12, FANCA and BBC3) are up-regulated and 6 genes (RAD1, CRY1, MSH2, ATRX, MCPH1 and RAD21) are down-regulated when cells were irradiated on spinach leaf scaffold (FIG. 15B). p53 signaling pathway is enriched by the upregulated genes while cell cycle is enriched by the downregulated genes, suggesting a potential switch in radiation-induced DNA damage signaling between leaf scaffold and stiff substrate. DNA double-strand breaks (DSBs) were investigaged by monitoring the formation of γH2AX foci. As shown on FIG. 15C, the number of γH2AX foci increased 1 h after 2 Gy-irradiation compared to sham-irradiated in both cells seeded on spinach scaffold or stiff substrate. At 6 h post-irradiation, the number of foci started to decrease to return to normal level at 24 h. Interestingly, the kinetic of DBSs restoration is similar between cells seeded on spinach scaffold or stiff substrate suggesting that, although it may be by different pathways, DNA damage is repaired as efficiently on leaf scaffold as in standard plate model (FIG. 15D). Although these experiments were only performed on cancer cells, they showed that radiation response can be measured on cells seeded on leaf scaffold and that the molecular pathways involved in this response differ from standard cell culture models, showing that leaf scaffolds offer a compatible model to study the effect of radiation in complex mechanical environments.

Pharmaceutical Countermeasure Approach

Rapamycin is a small molecule inhibitor of mammalian target of rapamycin (mTOR). mTOR coordinates eukaryotic cell growth and metabolism with environmental inputs including nutrients and growth factors. Extensive research over the past two decades has established a central role for mTOR in regulating many fundamental cell processes, from protein synthesis to autophagy, and deregulated mTOR signaling is implicated in the progression of cancer and diabetes, as well as the aging process (32). As such, rapamycin has several analogs including everolimus that are already FDA-approved as cancer therapeutics for human use (33).

Interestingly, some studies have also shown that rapamycin exerts a profoundly protective effect on normal cells upon exposure to IR. In mice, several studies showed the protecting effect of pharmacological mTOR inhibition with rapamycin to IR. A recent study demonstrated that transient inhibition of mTORC1 signaling by rapamycin treatment consistently accelerated liver recovery from irradiation, which was evidenced by decreasing sinusoidal congestion and increasing ALB expression after irradiation (34). The protective role of rapamycin on irradiated livers might be mediated by decreasing cellular apoptosis and increasing autophagy. Delivery of rapamycin also reduces radiation-induced pulmonary fibrosis and significantly prolonged survival after lethal thoracic irradiation (35). Treatment with rapamycin resulted in inhibition of radiation-induced signaling downstream of mTOR and reduced expression of pro-fibrotic, pro-inflammatory, and senescence associated cytokines in irradiated lungs. mTOR inhibition increases the clonogenic capacity of primary human oral keratinocytes and their resident self-renewing cells by preventing stem cell senescence and radiation-induced mucositis development (36). This protective effect of rapamycin is mediated by the increase in expression of mitochondrial superoxide dismutase (MnSOD), and the consequent inhibition of ROS formation and oxidative stress. mTOR inhibition also protects from the loss of proliferative basal epithelial stem cells upon IR in vivo, thereby preserving the integrity of the oral mucosa and protecting from radiation-induced mucositis. Finally, rapamycin pretreatment also mitigates hematopoietic system from radiation injury in both mice bone marrow and extramedullary hematopoietic organs by improving genomic stability and increasing survival of hematopoietic stem and progenitor cells (37).

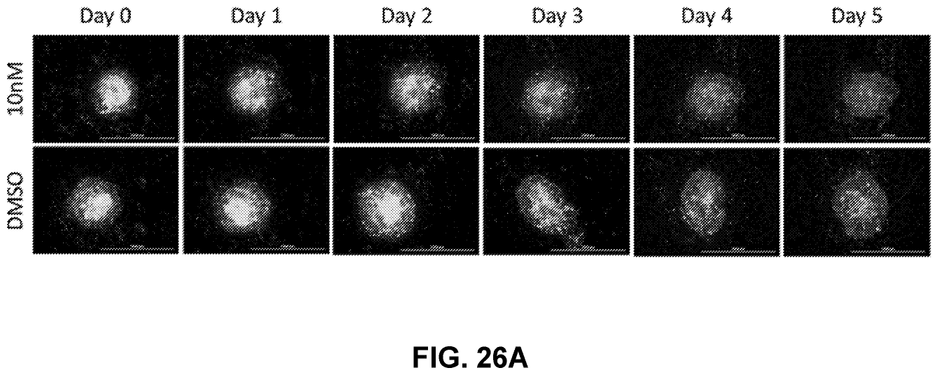

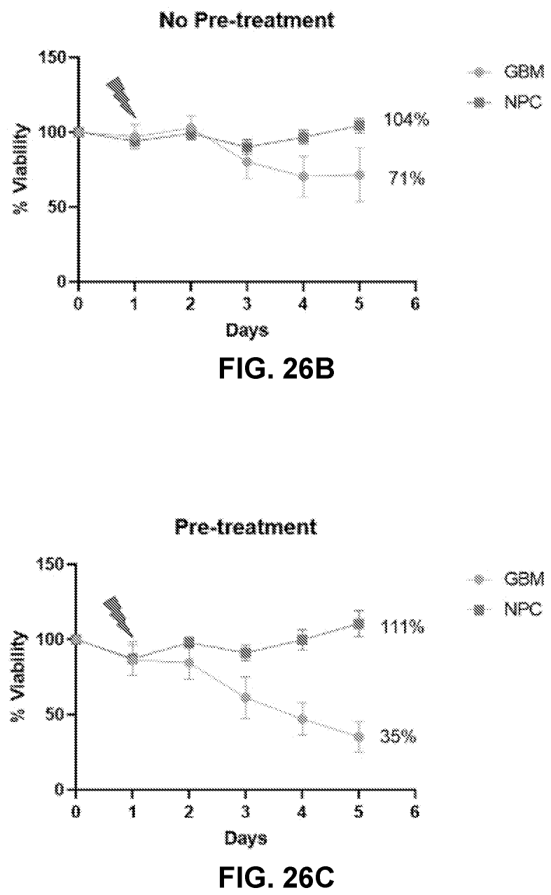

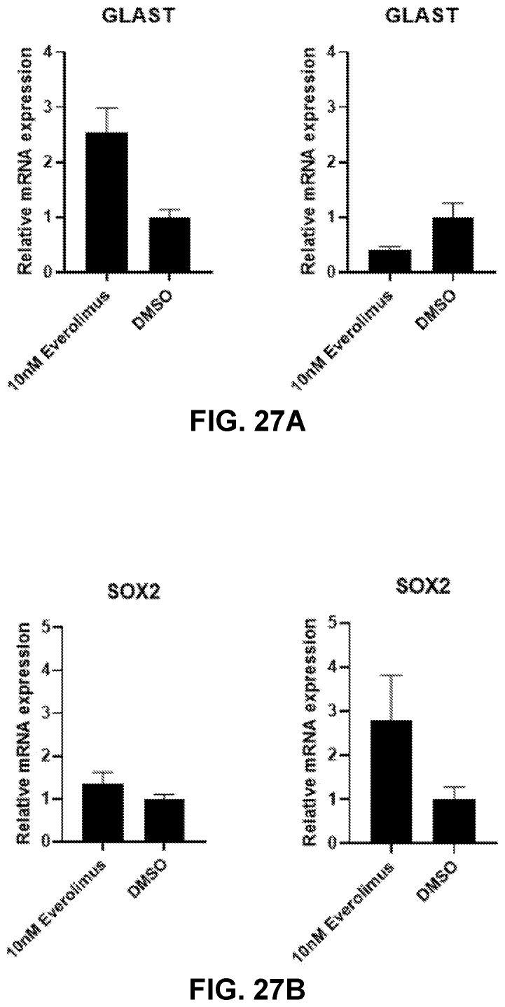

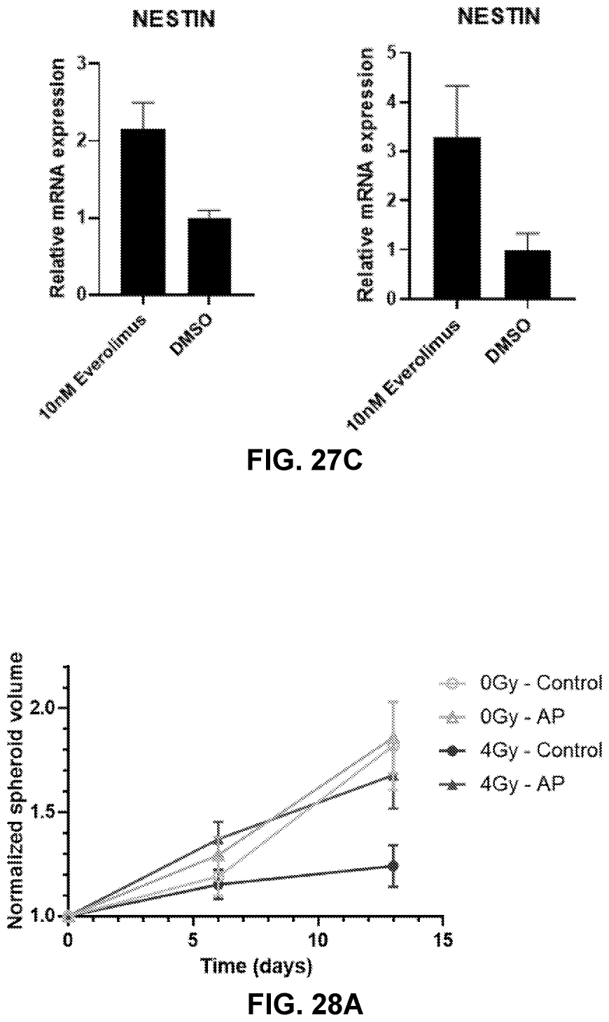

Thus, the effect of a rapamycin analog, everolimus, on human cells was investigated using an in vitro organoids model. GFP-tagged glioblastoma U87 cells were cultured in presence of RFP-tagged neural progenitor cells (NPCs), differentiated from dermal fibroblasts reprogrammed to induced pluripotent stem cells, to form organoids. The organoids were exposed to 10 nM everolimus for 24 hours before 2 Gy-irradiation. Results showed that for everolimus-treated organoids, GFP fluorescence intensity decreases over one week while RFP fluorescence signal increases compared to vehicle-treated organoids after irradiation (FIGS. 26A-26C), suggesting that U87 cells die upon radiation treatment whereas NPCs can survive and even proliferate. This has been confirmed by qRT-PCR where Glast mRNA level (glioblastoma biomarker) decreases whereas SOX2 and Nestin mRNA levels (NPCs biomarkers) increase in irradiated organoids pre-treatment with everolimus (FIGS. 27A-27C).

Non-Pharmaceutical/Non-Nutritional Radiation Countermeasure Approach

Complementary and alternative medicine, including acupuncture (AP), is gaining more and more interest. Several prospective trials in radiotherapy already investigated the effect of AP, mainly for the reduction of radiation-induced toxicity (38). Data are still controversial, some studies reporting benefits of true AP versus sham AP or standard treatment (39-41) while other showed no significance between true and sham AP (42,43). However, all of them showed significant improvement of AP over no treatment. Thus, without wishing to be bound by theory, it is believed that needle-generated stress could have an important effect on normal tissue response to external injuries.

How AP acts at cellular level is still poorly understood but multiples recent evidences suggest that the mechanoregulation response of the tissue could be of importance. Indeed, in studying AP, an important and frequently overlooked procedure is the manual needle manipulation performed by acupuncturists after needle insertion (44). The needle manipulations are typically a series of rapid bidirectional rotation or up-and-down piston movements. Spatially mapped and quantified by ultrasound, the needle stimulus is visualized to wind the tissue and cause tissue displacements of up to 100 μm (45). This deformation causes a mechanical signal that is sensed by nearby cells and convert into electrochemical activity and a biological response called mechanotransduction (46). Majority of the acupoint locations (80%) are found to be within connective tissue planes (47) and not along nervous channels as previously hypothesized (48). This leads to extracellular matrix rearrangements resulting in lower mechanical stress states of surrounding tissues (49). Using rat subcutaneous tissue explants, a study found that AP needle rotation caused fibroblasts to become aligned with collagen fibers and change shape from a rounded appearance to a more spindle-like shape (46). Increased cytoplasmic staining for polymerized F-actin can also be seen in fibroblasts one minute after needle rotation compared with needle insertion only. These observations suggest that the mechanical signal created by AP needle manipulation can induce intracellular cytoskeletal rearrangements in fibroblasts and possibly in other cells present within connective tissue, such as capillary endothelial cells. Cytoskeletal reorganization in response to mechanical load signals is known to induce cell contraction, migration, and protein synthesis as well as others potential effects, including autocrine and paracrine cellular effects (50,51). For instance, cytoskeleton also reorganizes after irradiation in endothelial cells (52) or mesenchymal stem cells (53) where it is involved in the increase of vascular permeability or senescence induction leading to radiation-induced irreversible damages. Autocrine and paracrine functions in bystander effects are well described and of importance for the transmission of radiation-induced damaging signals (54).

As direct evidence of the influence of mechanical stress on radiosensitivity, a study recently demonstrated that mechanical scratching occurring before IR decreases radiation-induced cell damage and increases cell repair in normal skin fibroblasts in vitro and in vivo (55). At the molecular level, this mechanical stress interrupts focal adhesion complexes and cell-cell cadherin interactions, transducing mechanical signals via activation of the phosphatidylinositol 3-kinase (PI3K), Akt, and glycogen synthase kinase 3 beta (GSK-3β) pathways. Although the mechanical injury applied to the cells in this study highlights for the first time that mechanical stress can protect skin cells from radiation.

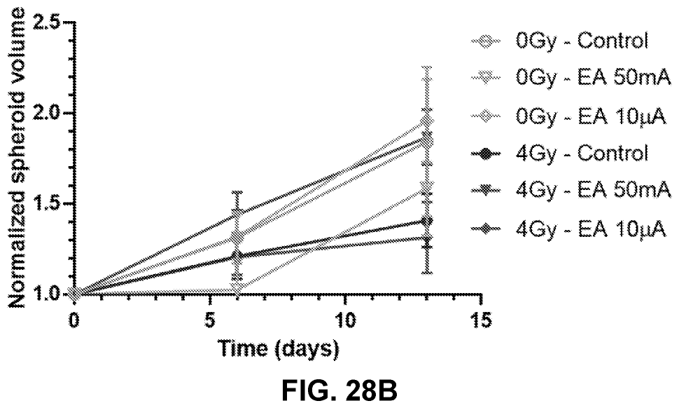

Thus, spheroids were generated with dermal fibroblasts+melanoma cell line that have been exposed to 4 Gy X-ray after being stimulated either by multiple standard AP needle punctures or by single electro-stimulated (EA) needle puncture. Results showed that after 2 weeks monitoring spheroid growth, volume of irradiated spheroids exposed to multiple puncture stimulation is higher than irradiated spheroids, suggesting that the needle-induced stress may have had a radioprotectant effect (FIG. 28A). In the non-irradiated group, AP-treated spheroids display the same volume as control spheroids, suggesting that AP treatment did not induce side effects. For the electro-stimulated spheroids, spheroids pre-treated with relevant physiological 50 mA current (and not with a 10 μA level) before irradiation present a higher volume after 15 days than non-treated spheroids (FIG. 28B). However, non-irradiated spheroids treated with 50 mA were smaller than non-treated spheroids suggesting that EA may slightly damage the spheroid.

Human skin is a complex living material, composed of several heterogeneous layers, each of them having different elasticity, stiffness, thickness, etc. Since these biomechanical properties regulate numerous biological processes through mechano-transduction mechanisms, including response to external stress (i.e. IR or drug exposure), there is a need to use a more comprehensive model reproducing both biochemical and mechanical complexity of human skin to evaluate the radiation effect. Thus, the response of skin cells, including keratinocytes and the deep layer of fibroblasts, as well as the epithelia-endothelial interaction, to IR was investigated by using a unique vascularized skin-on-leaf system that can recapitulate the mechanical properties of the multi-layer organization of the human skin. This system may be irradiated with low-LET photon (control) and high LET neutrons and radiation effects may be assessed by measuring inflammation response, DNA damages and cell integrity. In one embodiment, the multilayered vascularized biological system can be used to measure radiation effect on human skin and can provide a reliable platform for the assessment of skin countermeasure approaches.

Plant material useful for a multilayered vascularized biological system may include baby spinach leaves (Spinacia oleracea), hybrid cherry tomato plant leaves (Solanum lycopersicum), aquatic plant leaves (Echinodorus grisebachii) and A. borealis leaves (Kalanchoe fedtschenkoi variegati). To initiate decellularization, plant materials may be treated with serial chemical treatments including 5-min treatment with hexane to remove cuticle wax, followed by a solution of 1% sodium dodecyl sulfate (SDS) in deionized (DI) water for 48 hours, and a solution of 10% sodium chlorite and 0.1% Triton-X 100 in DI water for 48 hours. Finally, the leaves may be rinsed with DI water for an additional 2 days and then stored at 4° C. until use. Before starting biological experimental procedure, decellularized scaffolds may be individually selected in function of their stiffness as measured by AFM. AFM measurements may be performed at liquid interface with a Nanosurf Flex-Bio AFM System (Nanosurf, Switzerland) and gold coated cantilevers with nominal spring constant <0.1 N/m. Young's modulus may be extracted from the generated force curves using AtomicJ 1.7.2 software or equivalent software. The stiffest scaffolds may be used as the upper layer of epidermis (stratum corneum) and may not be recellularized. In a similar way, the softest may be used as the subcutaneous layer and may not be seeded with cells as well. Scaffold with intermediate stiffness may be used for epidermis (15-40 kPa) and dermis (50-90 kPa) with basal layers of each having the higher YM to better represent in vivo environment and YAP nuclear localization.

Prior to recellularization, each scaffold may be sterilized using a UV Stratalinker 2400 (Stratagene) for 30 minutes. Leaf scaffolds may be then functionalized by incubation in 50 ug/ml of collagen I in 20 mM acetic acid solution for 1 hour, followed by two washes in PBS and a final wash in complete medium. Finally, decellularized leaves may be cut into 1.5 cm×1.5 cm pieces to fit to the bottom of a non-treated 24-well tissue culture plate. 1.0 cm×1.0 cm inserts may be placed in the well on the top of the leaf to confine the cells and facilitate cellular attachment. Normal fibroblasts may be directly isolated from skin punch biopsy (from male and female healthy donors, matching average astronaut age) and seeded on 10 dermis layers. Some of them may be reprogrammed to iPSC by transfection of reprogramming transcription factors. The induced pluripotent stem cells may then differentiate into keratinocytes that may be seeded on five epidermis layers. Those human iPSC-derived SKPs (hiPSC-SKPs) show a similar gene expression signature to SKPs isolated from human skin dermis. Each seeded layer may be incubated alone for 24-48 hours to allow cell attachment and monolayer formation. Next, the full system may be assembled by superposing each layers to reach a thickness of ˜0.5 mm, which represents the average thickness of human skin (69). AFM measurement may be performed again on several skin-on-leaf systems to monitor the overall stiffness and thickness.

First, the system may be irradiated by using X-Ray as reference irradiation according to the NASA guidance in Human Explorations Research Opportunities (HERO). X-ray irradiation may be performed with X-RAD 320 cabinet (Precision X-Ray Inc.) at 320 KeV with a heavy 1.5 mm Al/0.75 mm Sn/0.25 mm Cu filter. The beam may be calibrated using a UNIDOS E PTW T10010 electrometer and TN30013 ionization chamber. Doses (0.5 and 1 Gy) may be delivered at a dose-rate <0.5 Gy/min.

After irradiation, the system may be “peeled” off and each layer may be collected at various time, including short (≤3 days) and long-time points (≥7 days). For the initial characterization, DNA damages from the different layers may be assessed by γH2AX/53BP1 foci assay at 1 h, 24 h, day 3 and day 5 post-irradiation. Analysis may be complemented by the measurement of DNA damage protein levels ((p)ATM, (p)DNA-Pkcs, Rad51, XRCC4, Ku70/Ku80, etc.) by western-blot (WB). Comparison between each layer may be made to highlight any “depth” effect due to the superposition of the layers and the three-dimensional conformation of the system. In addition, cell integrity, as measured by cell adhesion (E-Cadherin, integrin), tight junctions (occluding, ZO-1) or cytoskeleton modification (F-actin, Tau), may be assessed for short (1 and 3 days) and long timing points (5, 7 days, 2 and 4 weeks) by immunofluorescence (IF) or WB. Cellular death by apoptosis (TUNEL assay) and senescence (β-Galactosidase staining) may also be assessed for timing point >3 days. For a full view on the system, three skin-on-leaf of each condition (sham-irradiated vs. irradiated, for each time points) may be embedded with paraffin and sliced into 20 μm sections for IF staining. In vitro radiosensitivity of dermal fibroblasts is determined not only by processes directly involved in DNA-damage recognition and repair, but also by intracellular signaling cascades, which may lead to differentiation processes. For example, despite the presence of DNA damage in dermal fibroblasts weeks after the end of radiation treatment, there is no relationship between this damage and wound healing complications following surgery post-irradiation, suggesting that factors other than the radiosensitivity of the skin fibroblasts likely also play a role in radiation-induced skin injuries and wound healing (71). Therefore, in addition to “tissue” collection, supernatant may be regularly collected (1 h, 6 h, 12 h, day 1, 2, 3, 4, 5, 7, 14, 21 and 28) for reactive oxygen species (ROS) assay and cytokine profiles measurement. Total ROS as well as H₂O₂ and superoxide ion level may be measured. To assess the presence of any cytokine storm, levels may be measured using a multiplex human inflammatory cytokine ELISA Kit (Anogen, Canada) that includes IL-1α, IL-1β, IL-6, IL-8, GM-CSF, INF-γ, MCAF, and TNF-α. If necessary, cell lysis could also be collected to measure intracellular level of ROS and cytokines by flow cytometry.

Finally, emphasis may be given to the role of endothelial cells in radiation-induced skin injuries. For this, human primary dermal microvascular endothelial cells (HDMEC, PromoCell) that are Mayebrand Factor (vWF), CD31, Dil-Ac-LDL uptake positive, may be seeded into the vasculature of three additional dermis layers that may be added within the skin-on-leaf system. Biological outcomes similar to those previously described may be measured including DNA damages, skin cell integrity and death, inflammation response (ROS and cytokine assays). In addition, damages to endothelial cells may also be assessed. Mechanisms of action predominantly involve induction of a pro-inflammatory state by genotoxic stress, oxidative stress and damage-associated molecular patterns (DAMP) release that lead to pro-inflammatory state, procoagulatory and prothrombotic phenotype, mitochondrial dysfunction, and endothelial cell retraction and death (72). As such, in addition to bioassays previously described, endothelial adhesion molecules ICAM-1, VCAM-1, E-selectin and VE-cadherin expression levels, HMGB1 release, NO production and Ca²⁺ concentrations may be assessed in the vascular layers.

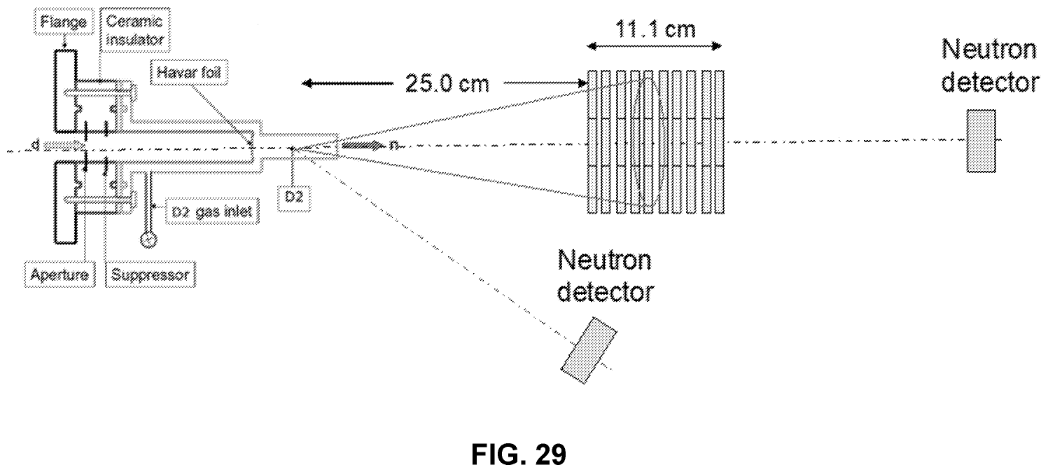

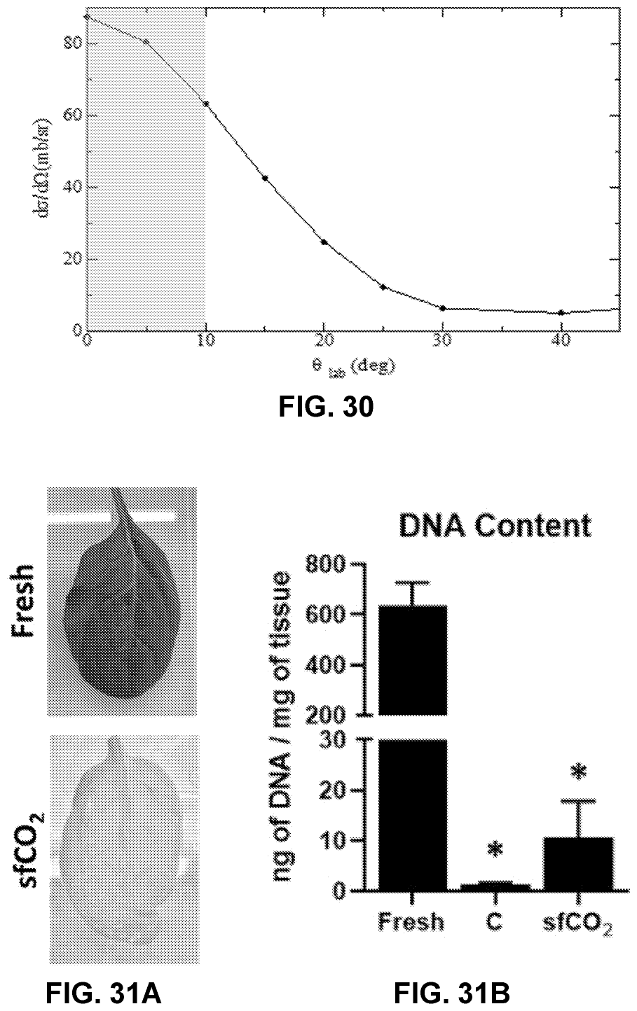

Once the system would have been characterized with X-Ray control, neutron irradiation may commence. The experiment layout for the sample irradiations is shown in FIG. 29. The neutrons are produced using the ²H(d, n)³He reaction by bombarding a 3.16-cm long gas cell that is pressurized to 7.8 atm. with research-grade deuterium gas. The gas is contained in the cell by a 6.35 μm thick Havar foil, and the deuteron beam is stopped in a 0.5 mm thick tantalum disk at the end of the cell. The incident deuteron beam energy is set to produce 10-MeV neutrons at the center of the gas cell. The angular distribution of the neutrons emitted from the source reaction for 10-MeV neutrons is shown in FIG. 30. The shaded region indicates the angular coverage of the samples during irradiation. The neutron flux decreases by 30% between the samples located in the center of the stack to those at the outer edge. The average dose of the samples in the center position may be about 15% higher than the dose in samples positioned in the ring around the center. The variation in the dose delivered to the samples in ring is estimated to be less than 5%. After 50% of the target dose has been delivered to the samples at the front of the stack, the stack may be rotated by 180° to even out the dose throughout the sample during the second half of the irradiation. The neutron beam flux may be monitored continuously during the irradiation by the two 2-in diameter×2-in thick liquid scintillation detectors.

To minimize background neutrons in the beam, each irradiation set may start with a new tantalum beam stop and a new Havar foil beam entrance window in the neutron production gas cell. This feature may be achieved by having foil inserts and tantalum beam stops fabricated solely for use in this project. The energy spectrum of the neutron beam may be measured at the beginning and end of each irradiation set. The spectrum may be used to calculate the absorbed radiation dose from the measured neutron beam flux. The dose rate is calculated using the equation: \dot{D}ₙ(E)=(Ø(E)EΣNᵢσ_{if}) where Dₙ is the dose rate (Gy/hr), Ø(E) is the neutron flux on the sample (neutrons/cm²/hr), E is neutron energy, Nᵢ is the number of nuclei of type i in the sample per unit of mass (kg), σᵢ is the scattering cross section of the i^{th} nucleus for neutrons of energy E, and f is the mean fractional energy transferred from a neutron to an atom during a collision. The f was computed using a Monte-Carlo simulation. The equation was evaluated using the elemental composition of synthetic human tissue (by mass: 71.4% oxygen, 14.9% carbon, 10.0% hydrogen, 3.4% nitrogen, 0.2% sodium and 0.1% chlorine) (73). The neutron-scattering cross sections, σᵢ, are taken from (74). For 10-MeV neutrons using the setup shown in FIG. 29, the calculated exposure field at the front of the center sample is 0.1635 mGy/hr/nA. With 3.0 μA of deuteron beam on target, the estimated dosage rate at the front and center of the sample stack may be 491 mGy/hr. Along the beam axis at the middle of the stack the dose is about (491 mGy/hr)×(25.0/30.5)²×0.75=247 mGy/hr. The dose is reduced because of the increased distance from the neutron source (25.0 to 30.5 cm) and because of the attenuation of the neutrons in the sample and disk materials (the transmission of 10-MeV neutrons through five samples is 0.75).

Biological outcome measured after neutron exposure may be identical to those measured for X-Ray irradiation and that may have demonstrated interest for characterizing our platform. One advantage of the skin-on-leaf system is to reproduce more accurately the mechanical constraints of the skin. Therefore, the mechanotransduction response to irradiation may be investigated. Recent studies showed that YAP and TAZ regulate skin wound healing by modulating the expression of TGF-β1 signaling pathway components such as Smad-2, p21, and Smad-7, and integrin signaling to control skin homeostasis, suggesting that YAP and TAZ localization to the nucleus is required for skin wound healing (31,75). Therefore, YAP/TAZ may be investigated through the different layers. WB will be used to assess protein expression from Hippo signaling pathway (including TEAD, LATS and 14-3-3) and IF to for subcellular localization of YAP/TAZ. YAP/TAZ activity will be assessed by measuring YAP target genes (CYR61, CTGF, AREG, MYC, Gli2, and AXL) using qRT-PCR (76).

Finally, multi-enzyme shotgun proteomics strategies developed by the National Cancer Institute's Clinical Proteomic Tumor Analysis Consortium (CPTAC) (77,78) may be used to identify protein changes after high LET irradiation within epidermis and dermis cells. At short- (day 1) and long-term (day 7) post-irradiation, keratinocyte, fibroblast and endothelial cells may be lyzed and fractionated by off-line high-pH chromatography (HpH) before subsequent LC-MS/MS analysis. Expression changes of proteins of interest may be confirmed using WB or ELISA-based method.

Of course, additional or alternative plants could be used to tune mechanical properties of the skin-on-leaf system. Mechanical properties of skin are also very heterogenous and vary with age, gender, ethnicity, anatomical location, hydration level, etc. However, the relative stiffness between each layer is reproducible and often match the same pattern, therefore the system may be assembled according to the following order of stiffness subcutaneous<epidermis<dermis<stratum corneum.

Measuring the Efficacy of a Pharmaceutical Drug to Protect Both Skin and Vascular Cells Against High LET Radiation Exposure

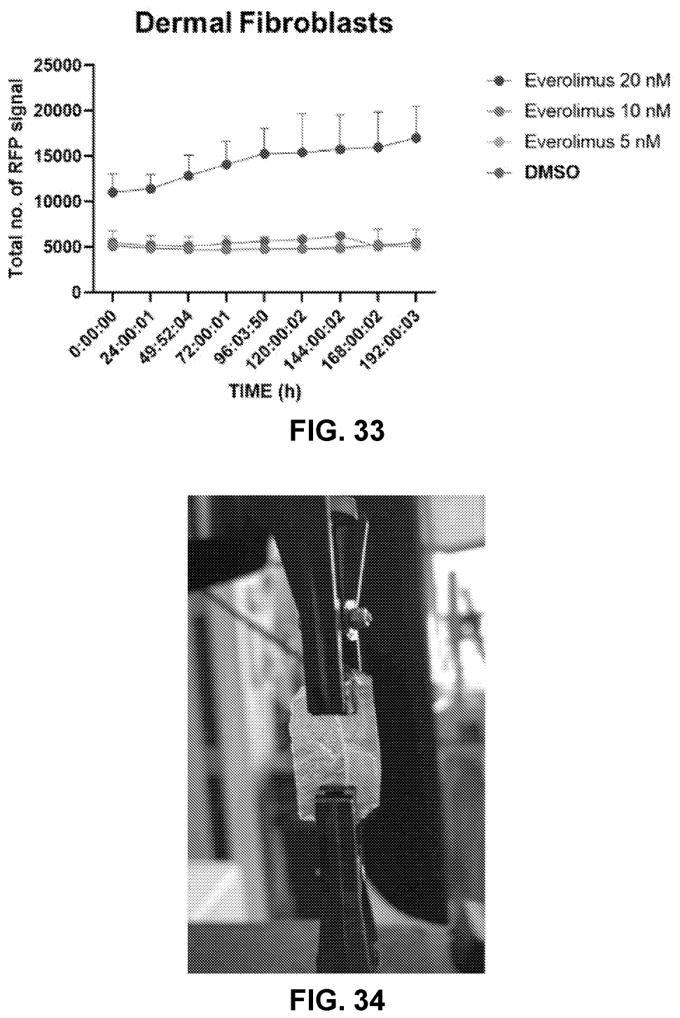

Response of Sk-MEL-28 cells to withaferin A (WFA), a natural compound from the withanolide family that induces apoptosis in human melanoma cell lines (96) and decreases uveal melanoma tumor growth in vivo (97) was assessed and showed that SK-MEL-28 cells seeded on stiff substrate (TCPS) were more sensitive to WFA (IC₅₀=1.1 μM) than cells on leaf scaffold (IC₅₀=5.2 μM) (FIG. 32), suggesting that the leaf model could better reproduce in vivo response. In addition, we previously showed that everolimus could protect neural progenitor stems from IR. We also assessed its effect on skin cells and demonstrated that the growth of irradiated dermal fibroblasts spheroids pre-treated with everolimus increased compared to the non-pretreated spheroids (FIG. 33), also suggesting a radioprotective of everolimus on skin cells.

Investigation of Acupuncture-Based Mechanical Stress as a New Radiation Countermeasure Approach

Without wishing to be bound by theory, it is believed that mechanical-induced stress of acupuncture triggers a radioprotective effect, regulated by the modification of cell adhesion contacts that activates mechanotransduction pathways and cytoskeletal rearrangements. Accordingly, the skin on a leaf structure may be used to investigate Acupuncture-based mechanical stress as a radiation countermeasure. The unique malleable and elastic characteristics of the sink on a leaf model allows an AP needle to be plunged deep into the dermis layer to mechanically stress the fibroblasts. The model may be then irradiated and inflammation response, as well as skin integrity and vascular functions, may be investigated to determine if such treatment resulted in less damage after irradiation compared to the control.

### REFERENCES ASSOCIATED WITH EXAMPLE 6

1. J. C. Chancellor, R. S. Blue, K. A. Cengel, S. M. Auñógn-Chancellor, K. H. Rubins, H. G. Katzgraber, A. R. Kennedy, Limitations in predicting the space radiation health risk for exploration astronauts. NPJ Microgravity. 4 (2018), doi:10.1038/s41526-018-0043-2.
2. M.-H. Y. Kim, K. A. George, F. A. Cucinotta, Evaluation of skin cancer risk for lunar and Mars missions. Adv. Space Res. 37, 1798-1803 (2006).
3. E. S. Place, N. D. Evans, M. M. Stevens, Complexity in biomaterials for tissue engineering. Nat. Mater. 8, 457-470 (2009).
4. S. Iravani, R. S. Varma, Plants and plant-based polymers as scaffolds for tissue engineering. Green Chem. 21, 4839-4867 (2019).
5. M. Adamski, G. Fontana, J. R. Gershlak, G. R. Gaudette, H. D. Le, W. L. Murphy, Two Methods for Decellularization of Plant Tissues for Tissue Engineering Applications. J. Vis. Exp. JoVE (2018), doi:10.3791/57586.
6. R. J. Hickey, A. E. Pelling, Cellulose Biomaterials for Tissue Engineering. Front. Bioeng. Biotechnol. 7 (2019), doi:10.3389/fbioe.2019.00045.
7. D. J. Modulevsky, C. M. Cuerrier, A. E. Pelling, Biocompatibility of Subcutaneously Implanted Plant-Derived Cellulose Biomaterials. PLOS ONE. 11, e0157894 (2016).
8. D. J. Modulevsky, C. Lefebvre, K. Haase, Z. Al-Rekabi, A. E. Pelling, Apple Derived Cellulose Scaffolds for 3D Mammalian Cell Culture. PLOS ONE. 9, e97835 (2014).
9. J. R. Gershlak, S. Hernandez, G. Fontana, L. R. Perreault, K. J. Hansen, S. A. Larson, B. Y. K. Binder, D. M. Dolivo, T. Yang, T. Dominko, M. W. Rolle, P. J. Weathers, F. Medina-Bolivar, C. L. Cramer, W. L. Murphy, G. R. Gaudette, Crossing kingdoms: Using decellularized plants as perfusable tissue engineering scaffolds. Biomaterials. 125, 13-22 (2017).
10. G. Fontana, J. Gershlak, M. Adamski, J.-S. Lee, S. Matsumoto, H. D. Le, B. Binder, J. Wirth, G. Gaudette, W. L. Murphy, Biofunctionalized Plants as Diverse Biomaterials for Human Cell Culture. Adv. Healthc. Mater. 6 (2017), doi:10.1002/adhm.201601225.
11. J. Read, G. D. Sanson, Characterizing sclerophylly: the mechanical properties of a diverse range of leaf types. New Phytol. 160, 81-99 (2003).
12. S. Wang, L. Ren, Y. Liu, Z. Han, Y. Yang, Mechanical characteristics of typical plant leaves. J. Bionic Eng. 7, 294-300 (2010).
13. L. J. Gibson, M. F. Ashby, B. A. Harley, Cellular materials in nature and medicine (2010).
14. N. Fekete, A. V. Boland, K. Campbell, S. L. Clark, C. A. Hoesli, Bags versus flasks: a comparison of cell culture systems for the production of dendritic cell-based immunotherapies. Transfusion (Paris). 58, 1800-1813 (2018).
15. Y. Yang, K. Wang, X. Gu, K. W. Leong, Biophysical Regulation of Cell Behavior—Cross Talk between Substrate Stiffness and Nanotopography. Engineering. 3, 36-54 (2017).
16. A. M. Handorf, Y. Zhou, M. A. Halanski, W.-J. Li, Tissue Stiffness Dictates Development, Homeostasis, and Disease Progression. Organogenesis. 11, 1-15 (2015).
17. T. R. Cox, J. T. Erler, Remodeling and homeostasis of the extracellular matrix: implications for fibrotic diseases and cancer. Dis. Model. Mech. 4, 165-178 (2011).
18. A. Skardal, D. Mack, A. Atala, S. Soker, Substrate elasticity controls cell proliferation, surface marker expression and motile phenotype in amniotic fluid-derived stem cells. J. Mech. Behav. Biomed. Mater. 17, 307-316 (2013).
19. X. Liang, S. A. Boppart, Biomechanical Properties of In Vivo Human Skin From Dynamic Optical Coherence Elastography. IEEE Trans. Biomed. Eng. 57, 953-959 (2010).
20. I. L. Kruglikov, P. E. Scherer, Skin aging as a mechanical phenomenon: The main weak links. Nutr. Healthy Aging. 4, 291-307.
21. S. Dikici, F. Claeyssens, S. MacNeil, Decellularised baby spinach leaves and their potential use in tissue engineering applications: Studying and promoting neovascularisation. J. Biomater. Appl. 34, 546-559 (2019).
22. Report No. 106—Limit for Exposure to “Hot Particles” on the Skin (1989)|NCRP|Bethesda, Md., (available at https://ncrponline.org/shop/reports/report-no-106-limit-for-exposure-to-hot-particles-on-the-skin-1989/).
23. V. Demidov, X. Zhao, O. Demidova, H. Y. M. Pang, C. Flueraru, F.-F. Liu, I. A. Vitkin, Preclinical quantitative in-vivo assessment of skin tissue vascularity in radiation-induced fibrosis with optical coherence tomography. J. Biomed. Opt. 23, 106003 (2018).
24. H. A. Van Den Brenk, The effect of ionizing radiations on capillary sprouting and vascular remodelling in the regenerating repair blastema observed in the rabbit ear chamber. Am. J. Roentgenol. Radium Ther. Nucl. Med. 81, 859-884 (1959).
25. S. P. Stearner, E. J. B. Christian, Mechanisms of Acute Injury in the γ-Irradiated Chicken In Vivo Studies of Dose Protraction Effects on the Microvasculature. Radiat. Res. 47, 741-755 (1971).
26. M. N. Andalib, Y. Dzenis, H. J. Donahue, J. Y. Lim, Biomimetic substrate control of cellular mechanotransduction. Biomater. Res. 20, 11 (2016).
27. M. Cantini, H. Donnelly, M. J. Dalby, M. Salmeron-Sanchez, The Plot Thickens: The Emerging Role of Matrix Viscosity in Cell Mechanotransduction. Adv. Healthc. Mater. n/a, 1901259.
28. A. Totaro, T. Panciera, S. Piccolo, YAP/TAZ upstream signals and downstream responses. Nat. Cell Biol. 20, 888-899 (2018).
29. E. Rognoni, G. Walko, The Roles of YAP/TAZ and the Hippo Pathway in Healthy and Diseased Skin. Cells. 8, 411 (2019).
30. G. Walko, S. Woodhouse, A. O. Pisco, E. Rognoni, K. Liakath-Ali, B. M. Lichtenberger, A. Mishra, S. B. Telerman, P. Viswanathan, M. Logtenberg, L. M. Renz, G. Donati, S. R. Quist, F. M. Watt, A genome-wide screen identifies YAP/WBP2 interplay conferring growth advantage on human epidermal stem cells. Nat. Commun. 8, 1-16 (2017).
31. M.-J. Lee, M. R. Byun, M. Furutani-Seiki, J.-H. Hong, H.-S. Jung, YAP and TAZ Regulate Skin Wound Healing. J. Invest. Dermatol. 134, 518-525 (2014).
32. R. A. Saxton, D. M. Sabatini, mTOR Signaling in Growth, Metabolism, and Disease. Cell. 168, 960-976 (2017).
33. R. Roskoski, Properties of FDA-approved small molecule protein kinase inhibitors. Pharmacol. Res. 144, 19-50 (2019).
34. W. Yang, L. Shao, S. Zhu, H. Li, X. Zhang, C. Ding, X. Wu, R. Xu, M. Yue, J. Tang, B. Kuang, G. Fan, Q. Zhu, H. Zeng, Transient Inhibition of mTORC1 Signaling Ameliorates Irradiation-Induced Liver Damage. Front. Physiol. 10 (2019), doi:10.3389/fphys.2019.00228.
35. E. J. Chung, A. Sowers, A. Thetford, G. McKay-Corkum, S. I. Chung, J. B. Mitchell, D. E. Citrin, mTOR inhibition with rapamycin mitigates radiation-induced pulmonary fibrosis in a murine model. Int. J. Radiat. Oncol. Biol. Phys. 96, 857-866 (2016).
36. R. Iglesias-Bartolome, V. Patel, A. Cotrim, K. Leelahavanichkul, A. A. Molinolo, J. B. Mitchell, J. S. Gutkind, mTOR Inhibition Prevents Epithelial Stem Cell Senescence and Protects from Radiation-Induced Mucositis. Cell Stem Cell. 11, 401-414 (2012).
37. Z. Wang, Y. Fang, M. Nie, N. Yuan, J. Wang, S. Zhang, Pre-treatment with rapamycin protects hematopoiesis against radiation injury. Int. J. Radiat. Res. 16, 65-74 (2018).
38. R. Asadpour, Z. Meng, K. A. Kessel, S. E. Combs, Use of acupuncture to alleviate side effects in radiation oncology: Current evidence and future directions. Adv. Radiat. Oncol. 1, 344-350 (2016).
39. M. K. Garcia, Z. Meng, D. I. Rosenthal, Y. Shen, M. Chambers, P. Yang, Q. Wei, C. Hu, C. Wu, W. Bei, S. Prinsloo, J. Chiang, G. Lopez, L. Cohen, Effect of True and Sham Acupuncture on Radiation-Induced Xerostomia Among Patients With Head and Neck Cancer: A Randomized Clinical Trial. JAMA Netw. Open. 2, e1916910-e1916910 (2019).
40. Z. Meng, M. K. Garcia, C. Hu, J. Chiang, M. Chambers, D. I. Rosenthal, H. Peng, Y. Zhang, Q. Zhao, G. Zhao, L. Liu, A. Spelman, J. L. Palmer, Q. Wei, L. Cohen, Randomized controlled trial of acupuncture for prevention of radiation-induced xerostomia among patients with nasopharyngeal carcinoma. Cancer. 118, 3337-3344 (2012).
41. Z. Meng, M. Kay Garcia, C. Hu, J. Chiang, M. Chambers, D. I. Rosenthal, H. Peng, C. Wu, Q. Zhao, G. Zhao, L. Liu, A. Spelman, J. Lynn Palmer, Q. Wei, L. Cohen, Sham-controlled, randomised, feasibility trial of acupuncture for prevention of radiation-induced xerostomia among patients with nasopharyngeal carcinoma. Eur. J. Cancer Oxf. Engl. 1990. 48, 1692-1699 (2012).
42. M. Blom, I. Dawidson, J. O. Fernberg, G. Johnson, B. Angmar-Månsson, Acupuncture treatment of patients with radiation-induced xerostomia. Eur. J. Cancer. B. Oral Oncol. 32B, 182-190 (1996).
43. A. Enblom, A. Johnsson, M. Hammar, E. Onelöv, G. Steineck, S. Börjeson, Acupuncture compared with placebo acupuncture in radiotherapy-induced nausea—a randomized controlled study. Ann. Oncol. Off. J. Eur. Soc. Med. Oncol. 23, 1353-1361 (2012).
44. G. Li, J.-M. Liang, P.-W. Li, X. Yao, P. Z. Pei, W. Li, Q.-H. He, X. Yang, Q. C. C. Chan, P. Y. S. Cheung, Q. Y. Ma, S. K. Lam, P. Y. C. Cheng, E. S. Yang, Physiology and cell biology of acupuncture observed in calcium signaling activated by acoustic shear wave. Pflugers Arch. 462, 587-597 (2011).
45. H. M. Langevin, E. E. Konofagou, G. J. Badger, D. L. Churchill, J. R. Fox, J. Ophir, B. S. Garra, Tissue displacements during acupuncture using ultrasound elastography techniques. Ultrasound Med. Biol. 30, 1173-1183 (2004).
46. H. M. Langevin, D. L. Churchill, M. J. Cipolla, Mechanical signaling through connective tissue: a mechanism for the therapeutic effect of acupuncture. FASEB J. 15, 2275-2282 (2001).
47. H. M. Langevin, J. A. Yandow, Relationship of acupuncture points and meridians to connective tissue planes. Anat. Rec. 269, 257-265 (2002).
48. N. Maurer, H. Nissel, M. Egerbacher, E. Gornik, P. Schuller, H. Traxler, Anatomical Evidence of Acupuncture Meridians in the Human Extracellular Matrix: Results from a Macroscopic and Microscopic Interdisciplinary Multicentre Study on Human Corpses. Evid. Based Complement. Alternat. Med. (2019), doi:https://doi.org/10.1155/2019/6976892.
49. C. E. Liddle, R. E. Harris, Cellular Reorganization Plays a Vital Role in Acupuncture Analgesia. Med. Acupunct. 30, 15-20 (2018).
50. A. K. Harris, P. Wild, D. Stopak, Silicone rubber substrata: a new wrinkle in the study of cell locomotion. Science. 208, 177-179 (1980).
51. D. P. Spadone, Alterations in aortic endothelial cell morphology and cytoskeletal protein synthesis during cyclic tensional deformation. J. Vasc. Surg. 9, 508-509 (1989).
52. D. Gabryś, G. Tozer, K. Trott, Ch. Kanthou, 157. The effects of radiation on the cytoskeleton of human endothelial cells in relation to endothelial monolayer permeability. Rep. Pract. Oncol. Radiother. 8, S253-S254 (2003).
53. D. Wang, D.-J. Jang, Protein kinase CK2 regulates cytoskeletal reorganization during ionizing radiation-induced senescence of human mesenchymal stem cells. Cancer Res. 69, 8200-8207 (2009).
54. V. N. Ivanov, H. Zhou, S. A. Ghandhi, T. B. Karasic, B. Yaghoubian, S. A. Amundson, T. K. Hei, Radiation-induced bystander signaling pathways in human fibroblasts: a role for interleukin-33 in the signal transmission. Cell. Signal. 22, 1076-1087 (2010).
55. Z. Chen, X. Wang, T. Jin, Y. Wang, C. S. Hong, L. Tan, T. Dai, L. Wu, Z. Zhuang, C. Shi, Increase in the radioresistance of normal skin fibroblasts but not tumor cells by mechanical injury. Cell Death Dis. 8, e2573 (2017).
56. C. Mothersill, R. Smith, M. Henry, C. Seymour, R. Wong, Alternative medicine techniques have non-linear effects on radiation response and can alter the expression of radiation induced bystander effects. Dose-Response Publ. Int. Hormesis Soc. 11, 82-98 (2013).
57. T.-J. Ho, T.-M. Chan, L.-I. Ho, C.-Y. Lai, C.-H. Lin, I. Macdonald, H.-J. Harn, J.-G. Lin, S.-Z. Lin, Y.-H. Chen, The possible role of stem cells in acupuncture treatment for neurodegenerative diseases: a literature review of basic studies. Cell Transplant. 23, 559-566 (2014).
58. T. E. Salazar, M. R. Richardson, E. Beli, M. S. Ripsch, J. George, Y. Kim, Y. Duan, L. Moldovan, Y. Yan, A. Bhatwadekar, V. Jadhav, J. A. Smith, S. McGorray, A. L. Bertone, D. O. Traktuev, K. L. March, L. M. Colon-Perez, K. G. Avin, E. Sims, J. A. Mund, J. Case, X. Deng, M. S. Kim, B. McDavitt, M. E. Boulton, J. Thinschmidt, S. L. Calzi, S. D. Fitz, R. K. Fuchs, S. J. Warden, T. McKinley, A. Shekhar, M. Febo, P. L. Johnson, L.-J. Chang, Z. Gao, M. G. Kolonin, S. Lai, J. Ma, X. Dong, F. A. White, H. Xie, M. C. Yoder, M. B. Grant, Electroacupuncture Promotes Central Nervous System-Dependent Release of Mesenchymal Stem Cells. STEM CELLS. 35, 1303-1315 (2017).
59. H. Cheng, J. Yu, Z. Jiang, X. Zhang, C. Liu, Y. Peng, F. Chen, Y. Qu, Y. Jia, Q. Tian, C. Xiao, Q. Chu, K. Nie, B. Kan, X. Hu, J. Han, Acupuncture improves cognitive deficits and regulates the brain cell proliferation of SAMP8 mice. Neurosci. Lett. 432, 111-116 (2008).
60. Q. Liu, J. Yu, W.-L. Mi, Q.-L. Mao-Ying, R. Yang, Y.-Q. Wang, G.-C. Wu, Electroacupuncture attenuates the decrease of hippocampal progenitor cell proliferation in the adult rats exposed to chronic unpredictable stress. Life Sci. 81, 1489-1495 (2007).
61. J. Gao, S. Wang, X. Wang, C. Zhu, Electroacupuncture enhances cell proliferation and neuronal differentiation in young rat brains. Neurol. Sci. Off. J. Ital. Neurol. Soc. Ital. Soc. Clin. Neurophysiol. 32, 369-374 (2011).
62. Q. Yan, J. Ruan, Y. Ding, W. Li, Y. Li, Y. Zeng, Electro-acupuncture promotes differentiation of mesenchymal stem cells, regeneration of nerve fibers and partial functional recovery after spinal cord injury. Exp. Toxicol. Pathol. Off. J. Ges. Toxikol. Pathol. 63, 151-156 (2011).
63. S. Moldenhauer, M. Burgauner, R. Hellweg, A. Lun, M. Hohenböken, E. Dietz, H. Kiesewetter, A. Salama, A. Moldenhauer, Mobilization of CD133(+)CD34(−) cells in healthy individuals following whole-body acupuncture for spinal cord injuries. J. Neurosci. Res. 88, 1645-1650 (2010).
64. R. P. Coppes, A. van der Goot, I. M. A. Lombaert, Stem Cell Therapy to Reduce Radiation-Induced Normal Tissue Damage. Semin. Radiat. Oncol. 19, 112-121 (2009).
65. M. Benderitter, F. Caviggioli, A. Chapel, R. P. Coppes, C. Guha, M. Klinger, O. Malard, F. Stewart, R. Tamarat, P. van Luijk, C. L. Limoli, Stem Cell Therapies for the Treatment of Radiation-Induced Normal Tissue Side Effects. Antioxid. Redox Signal. 21, 338-355 (2013).
66. T. G. Ebrahimian, F. Pouzoulet, C. Squiban, V. Buard, M. André, B. Cousin, P. Gourmelon, M. Benderitter, L. Casteilla, R. Tamarat, Cell therapy based on adipose tissue-derived stromal cells promotes physiological and pathological wound healing. Arterioscler. Thromb. Vasc. Biol. 29, 503-510 (2009).
67. M. Benderitter, P. Gourmelon, E. Bey, A. Chapel, I. Clairand, M. Prat, J. J. Lataillade, New emerging concepts in the medical management of local radiation injury. Health Phys. 98, 851-857 (2010).
68. W. A. Woodward, R. G. Bristow, Radiosensitivity of Cancer-Initiating Cells and Normal Stem Cells (or what the Heisenberg Uncertainly Principle has to do with Biology). Semin. Radiat. Oncol. 19, 87-95 (2009).
69. P. Oltulu, B. Ince, N. Kokbudak, S. Findik, F. Kilinc, Measurement of epidermis, dermis, and total skin thicknesses from six different body regions with a new ethical histometric technique. Turk. J. Plast. Surg. 26, 56 (2018).
70. M. Rave-Frank, P. Virsik-Köpp, O. Pradier, M. Nitsche, S. GrUnefeld, H. Schmidberger, In vitro response of human dermal fibroblasts to X-irradiation: relationship between radiation-induced clonogenic cell death, chromosome aberrations and markers of proliferative senescence or differentiation. Int. J. Radiat. Biol. 77, 1163-1174 (2001).
71. R. P. Hill, P. Kaspler, A. M. Griffin, B. O'Sullivan, C. Catton, H. Alasti, A. Abbas, M. Heydarian, P. Ferguson, J. S. Wunder, R. S. Bell, Studies of the in vivo radiosensitivity of human skin fibroblasts. Radiother. Oncol. J. Eur. Soc. Ther. Radiol. Oncol. 84, 75-83 (2007).
72. B. Baselet, P. Sonveaux, S. Baatout, A. Aerts, Pathological effects of ionizing radiation: endothelial activation and dysfunction. Cell. Mol. Life Sci. 76, 699-728 (2019).
73. G. L. Brownell, W. H. Ellett, A. R. Reddy, J. Nucl. Med. Off. Publ. Soc. Nucl. Med., in press.
74. ENDF: Evaluated Nuclear Data File, (available at https://www.nndc.bnl.gov/exfor/endf.htm).
75. A. Elbediwy, Z. I. Vincent-Mistiaen, B. Spencer-Dene, R. K. Stone, S. Boeing, S. K. Wculek, J. Cordero, E. H. Tan, R. Ridgway, V. G. Brunton, E. Sahai, H. Gerhardt, A. Behrens, I. Malanchi, O. J. Sansom, B. J. Thompson, Integrin signalling regulates YAP and TAZ to control skin homeostasis. Development. 143, 1674-1687 (2016).
76. M.-K. Kim, J.-W. Jang, S.-C. Bae, DNA binding partners of YAP/TAZ. BMB Rep. 51, 126-133 (2018).
77. P. Mertins, D. R. Mani, K. V. Ruggles, M. A. Gillette, K. R. Clauser, P. Wang, X. Wang, J. W. Qiao, S. Cao, F. Petralia, E. Kawaler, F. Mundt, K. Krug, Z. Tu, J. T. Lei, M. L. Gatza, M. Wilkerson, C. M. Perou, V. Yellapantula, K. Huang, C. Lin, M. D. McLellan, P. Yan, S. R. Davies, R. R. Townsend, S. J. Skates, J. Wang, B. Zhang, C. R. Kinsinger, M. Mesri, H. Rodriguez, L. Ding, A. G. Paulovich, D. Fenyö, M. J. Ellis, S. A. Carr, NCI CPTAC, Proteogenomics connects somatic mutations to signalling in breast cancer. Nature. 534, 55-62 (2016).
78. B. Zhang, J. Wang, X. Wang, J. Zhu, Q. Liu, Z. Shi, M. C. Chambers, L. J. Zimmerman, K. F. Shaddox, S. Kim, S. R. Davies, S. Wang, P. Wang, C. R. Kinsinger, R. C. Rivers, H. Rodriguez, R. R. Townsend, M. J. C. Ellis, S. A. Carr, D. L. Tabb, R. J. Coffey, R. J. C. Slebos, D. C. Liebler, NCI CPTAC, Proteogenomic characterization of human colon and rectal cancer. Nature. 513, 382-387 (2014).
79. P. M. Crapo, T. W. Gilbert, S. F. Badylak, An overview of tissue and whole organ decellularization processes. Biomaterials. 32, 3233-3243 (2011).
80. A. Gilpin, Y. Yang, Decellularization Strategies for Regenerative Medicine: From Processing Techniques to Applications. BioMed Res. Int. 2017 (2017), doi:10.1155/2017/9831534.
81. D. M. Casali, R. M. Handleton, T. Shazly, M. A. Matthews, A novel supercritical CO2-based decellularization method for maintaining scaffold hydration and mechanical properties. J. Supercrit. Fluids. 131, 72-81 (2018).
82. A. L. Hopkins, Network pharmacology: the next paradigm in drug discovery. Nat. Chem. Biol. 4, 682-690 (2008).
83. I. Kola, The state of innovation in drug development. Clin. Pharmacol. Ther. 83, 227-230 (2008).
84. R. Edmondson, J. J. Broglie, A. F. Adcock, L. Yang, Three-Dimensional Cell Culture Systems and Their Applications in Drug Discovery and Cell-Based Biosensors. Assay Drug Dev. Technol. 12, 207-218 (2014).
85. C. B. Gambacorti-Passerini, F. Rossi, M. Verga, H. Ruchatz, R. Gunby, R. Frapolli, M. Zucchetti, L. Scapozza, S. Bungaro, L. Tornaghi, F. Rossi, P. Pioltelli, E. Pogliani, M. D'Incalci, G. Corneo, Differences between in vivo and in vitro sensitivity to imatinib of Bcr/Abl+ cells obtained from leukemic patients. Blood Cells. Mol. Dis. 28, 361-372 (2002).
86. K. Shield, M. L. Ackland, N. Ahmed, G. E. Rice, Multicellular spheroids in ovarian cancer metastases: Biology and pathology. Gynecol. Oncol. 113, 143-148 (2009).
87. M. Zietarska, C. M. Maugard, A. Filali-Mouhim, M. Alam-Fahmy, P. N. Tonin, D. M. Provencher, A.-M. Mes-Masson, Molecular description of a 3D in vitro model for the study of epithelial ovarian cancer (EOC). Mol. Carcinog. 46, 872-885 (2007).
88. C. Wellbrock, S. Rana, H. Paterson, H. Pickersgill, T. Brummelkamp, R. Marais, Oncogenic BRAF Regulates Melanoma Proliferation through the Lineage Specific Factor MITF. PLOS ONE. 3, e2734 (2008).
89. L. A. Garraway, H. R. Widlund, M. A. Rubin, G. Getz, A. J. Berger, S. Ramaswamy, R. Beroukhim, D. A. Milner, S. R. Granter, J. Du, C. Lee, S. N. Wagner, C. Li, T. R. Golub, D. L. Rimm, M. L. Meyerson, D. E. Fisher, W. R. Sellers, Integrative genomic analyses identify MITF as a lineage survival oncogene amplified in malignant melanoma. Nature. 436, 117-122 (2005).
90. J. L. Simmons, C. J. Pierce, F. Al-Ejeh, G. M. Boyle, MITF and BRN2 contribute to metastatic growth after dissemination of melanoma. Sci. Rep. 7, 1-12 (2017).
91. S. Carreira, J. Goodall, L. Denat, M. Rodriguez, P. Nuciforo, K. S. Hoek, A. Testori, L. Larue, C. R. Goding, Mitf regulation of Dial controls melanoma proliferation and invasiveness. Genes Dev. 20, 3426-3439 (2006).
92. K. S. Hoek, 0. M. Eichhoff, N. C. Schlegel, U. Döbbeling, N. Kobert, L. Schaerer, S. Hemmi, R. Dummer, In vivo Switching of Human Melanoma Cells between Proliferative and Invasive States. Cancer Res. 68, 650-656 (2008).
93. M. Ennen, C. Keime, G. Gambi, A. Kieny, S. Coassolo, C. Thibault-Carpentier, F. Margerin-Schaller, G. Davidson, C. Vagne, D. Lipsker, I. Davidson, MITF-High and MITF-Low Cells and a Novel Subpopulation Expressing Genes of Both Cell States Contribute to Intra- and Intertumoral Heterogeneity of Primary Melanoma. Clin. Cancer Res. Off. J. Am. Assoc. Cancer Res. 23, 7097-7107 (2017).
94. S. Aida, Y. Sonobe, H. Tanimura, N. Oikawa, M. Yuhki, H. Sakamoto, T. Mizuno, MITF suppression improves the sensitivity of melanoma cells to a BRAF inhibitor. Cancer Lett. 409, 116-124 (2017).
95. J. Müller, O. Krijgsman, J. Tsoi, L. Robert, W. Hugo, C. Song, X. Kong, P. A. Possik, P. D. M. Cornelissen-Steijger, M. H. G. Foppen, K. Kemper, C. R. Goding, U. McDermott, C. Blank, J. Haanen, T. G. Graeber, A. Ribas, R. S. Lo, D. S. Peeper, Low MITF/AXL ratio predicts early resistance to multiple targeted drugs in melanoma. Nat. Commun. 5, 1-15 (2014).
96. E. Mayola, C. Gallerne, D. D. Esposti, C. Martel, S. Pervaiz, L. Larue, B. Debuire, A. Lemoine, C. Brenner, C. Lemaire, Withaferin A induces apoptosis in human melanoma cells through generation of reactive oxygen species and down-regulation of Bcl-2. Apoptosis Int. J. Program. Cell Death. 16, 1014-1027 (2011).
97. A. K. Samadi, S. M. Cohen, R. Mukerji, V. Chaguturu, X. Zhang, B. N. Timmermann, M. S. Cohen, E. A. Person, Natural withanolide withaferin A induces apoptosis in uveal melanoma cells by suppression of Akt and c-MET activation. Tumour Biol. J. Int. Soc. Oncodevelopmental Biol. Med. 33, 1179-1189 (2012).
98. L. M. Ballou, R. Z. Lin, Rapamycin and mTOR kinase inhibitors. J. Chem. Biol. 1, 27-36 (2008).
99. I. Eke, N. Cordes, Focal adhesion signaling and therapy resistance in cancer. Semin. Cancer Biol. 31, 65-75 (2015).
100. H. L. Goel, A. Sayeed, M. Breen, M. J. Zarif, D. S. Garlick, I. Leav, R. J. Davis, T. J. FitzGerald, A. Morrione, C.-C. Hsieh, Q. Liu, A. P. Dicker, D. C. Altieri, L. R. Languino, β1 integrins mediate resistance to ionizing radiation in vivo by inhibiting c-Jun amino terminal kinase 1. J. Cell. Physiol. 228, 1601-1609 (2013).
101. H. Tanaka, M. Kawaguchi, S. Shoda, T. Miyoshi, R. Iwasaki, F. Hyodo, T. Mori, A. Hara, H. Tomita, M. Matsuo, Nuclear Accumulation of β-Catenin in Cancer Stem Cell Radioresistance and Stemness in Human Colon Cancer. Anticancer Res. 39, 6575-6583 (2019).
102. M. S. Chen, W. A. Woodward, F. Behbod, S. Peddibhotla, M. P. Alfaro, T. A. Buchholz, J. M. Rosen, Wnt/beta-catenin mediates radiation resistance of Sca1+ progenitors in an immortalized mammary gland cell line. J. Cell Sci. 120, 468-477 (2007).
103. W. A. Woodward, M. S. Chen, F. Behbod, M. P. Alfaro, T. A. Buchholz, J. M. Rosen, WNT/beta-catenin mediates radiation resistance of mouse mammary progenitor cells. Proc. Natl. Acad. Sci. U.S.A. 104, 618-623 (2007).

### Example 7: Multilayered Vascularized Biological System to Study Melanoma

Melanoma is the malignancy of the pigment-producing melanocytes in the skin and characterized by its highly aggressive nature to metastasize to distant organs. Its prognosis remains poor, with a five-year survival rate of 16% for patients with distant metastases¹, and its incidence continues to rise year-over-year, with almost 97,000 new cases in the United States projected for 2019². As such, melanoma is a significant health issue and there is an urgent need to better understand underlying mechanisms of disease, to develop and test new therapeutics drugs for preventing progression. Currently, this is limited by the lack of reliable human in vitro models that are not able to mimic the complex human melanoma microenvironment by reproducing heterogeneity in both cellularity (epithelial, vascular, immune cells, and fibroblasts) and extracellular matrix (ECM) composition³. Despite important progress in organ-on-chip technologies, tissue engineering, bioengineered platforms and 3D printing, current models have many limitations including high inflammatory response, non-reproducible physical properties, poor fluid diffusion and low angiogenesis capability. Because of their unique architectural, biomechanical and biochemical properties, plant-based cellulose scaffolds can provide a promising alternative to overcome these challenges. In this application we will investigate the following aims:

In one embodiment, the stratified “skin-on-leaf” platform disclosed herein may recapitulate the damage patterns of UVA and UVB irradiation on human skin.

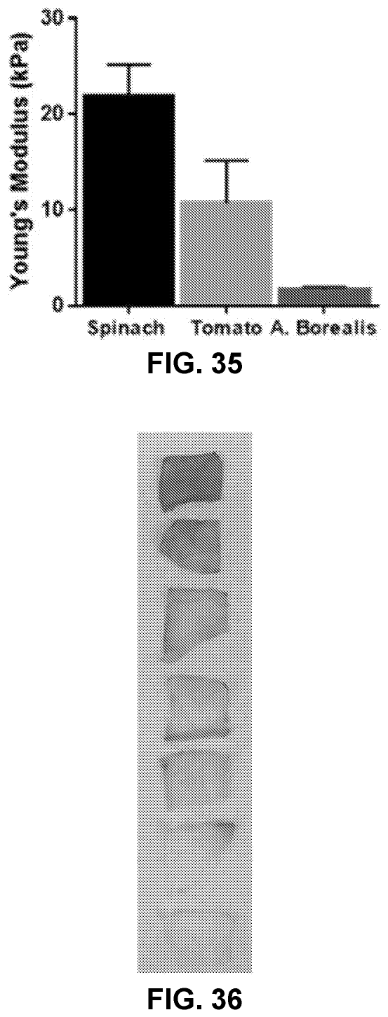

Different plant materials were decellularized to recreate a multi-layer skin model by using cellulose scaffolds from (1) stiff spinach leaves from the (stiffest) upper part (stratum corneum) of the epidermis, (2) tomato leaves from the epidermis, (3) soft spinach leaves for the dermis and (4) A. borealis for the (softest) subcutaneous layer (FIG. 35) to reach a thickness of ˜0.5 mm, which represents the average thickness of human skin¹⁴. In this model, human normal epidermal keratinocytes were seeded on tomato leaves and human normal fibroblasts on spinach leaves to provide the cellular components of epidermis and dermis respectively. Fluorescence microscopy imaging confirmed that cells were present on the leaf surface (F-actin), able to create tight junctions, as visualized by the expression of β-catenin, and survive up to four weeks in the system. Thus, this model reproduces the stratified skin structure by integrating its specific mechanical constraints and cellular components, making this system adapted for the study of complex response such as the effect of external stress on skin or for the screening and test of new therapeutic drugs.

Plant material may be decellularized by serial chemical treatments including 5-min treatment with hexane to remove cuticle wax, followed by a solution of 1% sodium dodecyl sulfate in deionized (DI) water for 48 hours, and a solution of 10% sodium chlorite and 0.1% Triton-X 100 in DI water for 48 hours. Finally, the leaves may be rinsed with DI water for an additional 2 days and then stored at 4° C. until use. Before starting biological experimental procedure, decellularized scaffolds may be individually selected in function of their stiffness as measured by AFM. Prior to recellularization, each scaffold may be sterilized using a UV Stratalinker 2400 (Stratagene) for 30 minutes. Finally, decellularized leaves may be cut into small pieces to fit to the bottom of a non-treated 24-well tissue culture plate. Normal dermal fibroblasts (BJ ATCC, CRL-2522) may be seeded on 10 dermis layers. BRAF wild type (Sk-mel-2) or mutant (Sk-mel-28) melanoma cells may be seeded on five epidermis layers. Each seeded layer may be incubated alone for 24-48 hours to allow cell attachment and monolayer formation. Next, the full system may be assembled by superposing each layer to reach a thickness of ˜0.5 mm. All cell lines share the same cell culture medium (EMEM supplemented with 10% fetal bovine serum). System may be then exposed to a concentration (pre-determined by dose-response relationship using conventional assay on standard cell culture model) of FDA-approved drugs PLX4032 (BRAFi) and Dacarbazine as well as DMSO (control) for 72 h. Cell proliferation and viability of melanoma cells may be assessed by immunofluorescence (Ki67, PCNA, TUNEL assay) and MTT assay. Fluorescence/colorimetric intensity values and counts may be used to interpret data. Results may compare the responses between BRAF wildtype and mutant melanoma cells and the different drugs. In addition, outcomes may also be compared to cells seeded in standard cell culture flasks and exposed to the drugs following the same protocol. To assess significance when comparing two groups, the Bartlett test may be first used to test if the values present significant homogeneous or heterogeneous variances between groups. Then, significance of two-group comparisons may be calculated using the Mann-Whitney nonparametric test (for heterogenous variances) or 2-tailed Student's t-test (homogeneous). P values of <0.05 may be considered significant.

The skin-on-leaf system will be assembled as described above, and then be exposed to UVA or UVB radiation. Dose may be pre-determined by assessment of the misrepair of UV-induced cyclobutane pyrimidine dimers (CPD) that are the most predominant, pre-mutagenic DNA-lesions produced by both UVB and UVA irradiation of human skin cells²⁶. Then, specific UV type damages may be assessed on each layers of the skin-on-leaf system, both in melanocyte and fibroblast cells. UVA is known to induce DNA damage indirectly through photooxidative mechanisms, Therefore, specific effect of UVA may be assessed by the detection of nuclear 8-Oxo-7,8-dihydro-2′-deoxyguanosine (8-oxodGuo) and 8-oxyguanine DNA glycosylase 1 (OGG1)²⁷. In addition, phosphorylation of AKT at Thr308, that is induced only by UVA but not by UVB, may also be assessed. Conversely, phosphorylation of JNK1/2 at Thr138/Tyr185 or STAT3 at Ser727, specific of UVB damages, may be assessed on both cell type. In addition, by using the unique fluid transport properties of the leaf system, we may assess the efficiency of sunscreen by exposing the upper layer to the active compounds (i.e. zinc oxide). The skin-on-leaf system has been shown to provide a diffusion gradient from the exposed upper layer to the different layers, thus mimicking skin absorption (FIG. 37). System with and without sunscreen pre-treatment may be then exposed to UV irradiation and same outcome as previously described may be collected. Signal may be interpreted as fluorescence intensity value, normalized to non-irradiated samples, and analyzed between the different groups (UVA vs. UVB; no-treatment vs. sunscreen treatment) following the same statistical approach as detailed in Aim #1.

### REFERENCES ASSOCIATED WITH EXAMPLE 7

1. Kuzu, O. F., Nguyen, F. D., Noory, M. A. & Sharma, A. Current State of Animal (Mouse) Modeling in Melanoma Research. Cancer Growth Metastasis 8, 81-94 (2015).
2. Siegel, R. L., Miller, K. D. & Jemal, A. Cancer statistics, 2019. CA. Cancer J. Clin. 69, 7-34 (2019).
3. Hanahan, D. & Weinberg, R. A. Hallmarks of cancer: the next generation. Cell 144, 646-674 (2011).
4. Place, E. S., Evans, N. D. & Stevens, M. M. Complexity in biomaterials for tissue engineering. Nat. Mater. 8, 457-470 (2009).
5. Iravani, S. & Varma, R. S. Plants and plant-based polymers as scaffolds for tissue engineering. Green Chem. 21, 4839-4867 (2019).
6. Adamski, M. et al. Two Methods for Decellularization of Plant Tissues for Tissue Engineering Applications. J. Vis. Exp. JoVE (2018) doi:10.3791/57586.
7. Hickey, R. J. & Pelling, A. E. Cellulose Biomaterials for Tissue Engineering. Front. Bioeng. Biotechnol. 7, (2019).
8. Modulevsky, D. J., Cuerrier, C. M. & Pelling, A. E. Biocompatibility of Subcutaneously Implanted Plant-Derived Cellulose Biomaterials. PLOS ONE 11, e0157894 (2016).
9. Modulevsky, D. J., Lefebvre, C., Haase, K., Al-Rekabi, Z. & Pelling, A. E. Apple Derived Cellulose Scaffolds for 3D Mammalian Cell Culture. PLOS ONE 9, e97835 (2014).
10. Gershlak, J. R. et al. Crossing kingdoms: Using decellularized plants as perfusable tissue engineering scaffolds. Biomaterials 125, 13-22 (2017).
11. Fontana, G. et al. Biofunctionalized Plants as Diverse Biomaterials for Human Cell Culture. Adv. Healthc. Mater. 6, (2017).
12. Liang, X. & Boppart, S. A. Biomechanical Properties of In Vivo Human Skin From Dynamic Optical Coherence Elastography. IEEE Trans. Biomed. Eng. 57, 953-959 (2010).
13. Kruglikov, I. L. & Scherer, P. E. Skin aging as a mechanical phenomenon: The main weak links. Nutr. Healthy Aging 4, 291-307.
14. Oltulu, P., Ince, B., Kokbudak, N., Findik, S. & Kilinc, F. Measurement of epidermis, dermis, and total skin thicknesses from six different body regions with a new ethical histometric technique. Turk. J. Plast. Surg. 26, 56 (2018).
15. Janmey, P. A., Fletcher, D. A. & Reinhart-King, C. A. Stiffness Sensing by Cells. Physiol. Rev. 100, 695-724 (2019).
16. Murray, M. E., Mendez, M. G. & Janmey, P. A. Substrate stiffness regulates solubility of cellular vimentin. Mol. Biol. Cell 25, 87-94 (2014).
17. Yeung, T. et al. Effects of substrate stiffness on cell morphology, cytoskeletal structure, and adhesion. Cell Motil. Cytoskeleton 60, 24-34 (2005).
18. Jannatbabaei, A., Tafazzoli-Shadpour, M., Seyedjafari, E. & Fatouraee, N. Cytoskeletal remodeling induced by substrate rigidity regulates rheological behaviors in endothelial cells. J. Biomed. Mater. Res. A 107, 71-80 (2019).
19. Lampi, M. C. & Reinhart-King, C. A. Targeting extracellular matrix stiffness to attenuate disease: From molecular mechanisms to clinical trials. Sci. Transl. Med. 10, (2018).
20. Tokuda, E. Y., Leight, J. L. & Anseth, K. S. Modulation of matrix elasticity with PEG hydrogels to study melanoma drug responsiveness. Biomaterials 35, 4310-4318 (2014).
21. Orgaz, J. L. et al. Myosin II Reactivation and Cytoskeletal Remodeling as a Hallmark and a Vulnerability in Melanoma Therapy Resistance. Cancer Cell 37, 85-103.e9 (2020).
22. Kalluri, R. & Zeisberg, M. Fibroblasts in cancer. Nat. Rev. Cancer 6, 392-401 (2006).
23. Flach, E. H., Rebecca, V. W., Herlyn, M., Smalley, K. S. M. & Anderson, A. R. A. Fibroblasts contribute to melanoma tumor growth and drug resistance. Mol. Pharm. 8, 2039-2049 (2011).
24. Tiago, M. et al. Fibroblasts Protect Melanoma Cells from the Cytotoxic Effects of Doxorubicin. Tissue Eng. Part A 20, 2412-2421 (2014).
25. Sample, A. & He, Y.-Y. Mechanisms and prevention of UV-induced melanoma. Photodermatol. Photoimmunol. Photomed. 34, 13-24 (2018).
26. Kraemer, A. et al. UVA and UVB Irradiation Differentially Regulate microRNA Expression in Human Primary Keratinocytes. PLOS ONE 8, e83392 (2013).
27. Noonan, F. P. et al. Melanoma induction by ultraviolet A but not ultraviolet B radiation requires melanin pigment. Nat. Commun. 3, 884 (2012).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). Appendix A is hereby incorporated by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroup, are disclosed separately. When a Markush group or other grouping is used herein, all individual members of the group and all combinations and subcombinations possible of the group are intended to be individually included in the disclosure.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of simulating a biological response of a cellular system, the method comprising:
removing at least some DNA-containing material from a vascular plant tissue to produce a vascularized cellulose scaffold;
seeding the vascularized cellulose scaffold with cultured biological cells;
growing the cultured biological cells on the vascularized cellulose scaffold to produce a vascularized biological system;
subjecting the vascularized biological system to an external stimulus; and
measuring a response of the vascularized biological system.

2. The method of claim 1, wherein the removing step comprises submerging the plant tissue in a fluid comprising supercritical CO₂.

3. The method of claim 2, wherein the fluid comprises peracetic acid and ethanol.

4. The method of claim 3, wherein the fluid comprises at least 80 wt % supercritical CO₂; and wherein the ratio of peracetic acid to ethanol is from 1:100 to 1:10.

5. The method of claim 1, wherein the external stimulus comprises a pharmacological compound.

6. The method of claim 1, wherein the external stimulus comprises ionizing radiation.

7. The method of claim 1, wherein the external stimulus comprises a mechanical tear or puncture.

8. The method of claim 1, wherein the external stimulus comprises a pesticide, a nerve agent, a corrosive liquid, or combinations thereof.

9. The method of claim 1, wherein the cultured biological cells comprise human cancer cells.

10. The method of claim 9, wherein the vascularized biological system recapitulates a tumor microenvironment.

11. The method of claim 1, wherein the removing step occurs at a temperature that is less than or equal to 40° C.

12. The method of claim 1, wherein the removing step occurs at a temperature from 25° C. to 38° C.

13. The method of claim 1, wherein the growing comprises supplying the cultured biological cells with a growth medium via vessels of the vascularized cellulose scaffold.

14. The method of claim 1, wherein the seeding step comprises seeding the vascularized cellulose scaffold with endothelial cells.

15. A method of simulating a biological response of a multilayer cellular system, the method comprising:
removing at least some DNA-containing material from a first vascular plant tissue to produce a first vascularized cellulose scaffold;
removing at least some DNA-containing material from a second vascular plant tissue to produce a second vascularized cellulose scaffold;
combining the first and second vascularized cellulose scaffolds to create a multilayered scaffold;
seeding the multilayered scaffold with cultured biological cells;
growing the cultured biological cells on the multilayer scaffold to produce a multilayered vascularized biological system;
subjecting the multilayered vascularized biological system to an external stimulus; and
measuring a response of the multilayered vascularized biological system.

16. The method of claim 15, wherein the seeding step comprises:
seeding a first layer of the multilayered scaffold with cultured biological cells of a first cell type; and
seeding a second layer of the multilayered scaffold with cultured biological cells of a second cell type.

17. The method of claim 15, wherein the multilayered scaffold comprises a third vascularized cellulose scaffold.

18. The method of claim 16, wherein the first cell type is keratinocytes and the second cell type is dermal fibroblasts.

19. The method of claim 18, wherein the first layer of the multilayered scaffold is derived from a tomato leaf and the second layer is derived from spinach leaves.

20. The method of claim 15, wherein the multilayered vascularized biological system is a living model of human skin.
